SEQUENCE LISTING (490 sequences)

SEQ ID NO: 1 - DNA, 21 bp, synthetic construct
  source 1..21: mol_type = other DNA
gacggccgctcaggttctgct

SEQ ID NO: 2 - DNA, 21 bp, synthetic construct
  source 1..21: mol_type = other DNA
gacggccgctcaggttctgtt

SEQ ID NO: 3 - DNA, 21 bp, synthetic construct
  source 1..21: mol_type = other DNA
acggccgctcaggttctgctt

SEQ ID NO: 4 - DNA, 21 bp, synthetic construct
  source 1..21: mol_type = other DNA
acggccgctcaggttctgttt

SEQ ID NO: 5 - DNA, 21 bp, synthetic construct
  source 1..21: mol_type = other DNA
cggccgctcaggttctgcttt

SEQ ID NO: 6 - DNA, 21 bp, synthetic construct
  source 1..21: mol_type = other DNA
cggccgctcaggttctgtttt

SEQ ID NO: 7 - DNA, 21 bp, synthetic construct
  source 1..21: mol_type = other DNA
ggccgctcaggttctgctttt

SEQ ID NO: 8 - DNA, 21 bp, synthetic construct
  source 1..21: mol_type = other DNA
ggccgctcaggttctgttttt

SEQ ID NO: 9 - DNA, 21 bp, synthetic construct
  source 1..21: mol_type = other DNA
gccgctcaggttctgctttta

SEQ ID NO: 10 - DNA, 21 bp, synthetic construct
  source 1..21: mol_type = other DNA
gccgctcaggttctgttttta

SEQ ID NO: 11 - DNA, 21 bp, synthetic construct
  source 1..21: mol_type = other DNA
ccgctcaggttctgcttttac

SEQ ID NO: 12 - DNA, 21 bp, synthetic construct
  source 1..21: mol_type = other DNA
ccgctcaggttctgtttttac

SEQ ID NO: 13 - DNA, 21 bp, synthetic construct
  source 1..21: mol_type = other DNA
cgctcaggttctgcttttacc

SEQ ID NO: 14 - DNA, 21 bp, synthetic construct
  source 1..21: mol_type = other DNA
cgctcaggttctgtttttacc

SEQ ID NO: 15 - DNA, 21 bp, synthetic construct
  source 1..21: mol_type = other DNA
gctcaggttctgcttttacct

SEQ ID NO: 16 - DNA, 21 bp, synthetic construct
  source 1..21: mol_type = other DNA
gctcaggttctgtttttacct

SEQ ID NO: 17 - DNA, 21 bp, synthetic construct
  source 1..21: mol_type = other DNA
ctcaggttctgcttttacctg

SEQ ID NO: 18 - DNA, 21 bp, synthetic construct
  source 1..21: mol_type = other DNA
ctcaggttctgtttttacctg

SEQ ID NO: 19 - DNA, 21 bp, synthetic construct
  source 1..21: mol_type = other DNA
tggctactaaatgtgctctta

SEQ ID NO: 20 - DNA, 21 bp, synthetic construct
  source 1..21: mol_type = other DNA
tggctactaaatgtgctgtta

SEQ ID NO: 21 - DNA, 21 bp, synthetic construct
  source 1..21: mol_type = other DNA
ggctactaaatgtgctgttag

SEQ ID NO: 22 - DNA, 21 bp, synthetic construct
  source 1..21: mol_type = other DNA
gctactaaatgtgctcttagg

SEQ ID NO: 23 - DNA, 21 bp, synthetic construct
  source 1..21: mol_type = other DNA
gctactaaatgtgctgttagg

SEQ ID NO: 24 - DNA, 21 bp, synthetic construct
  source 1..21: mol_type = other DNA
ctactaaatgtgctcttaggc

SEQ ID NO: 25 - DNA, 21 bp, synthetic construct
  source 1..21: mol_type = other DNA
ctactaaatgtgctgttaggc

SEQ ID NO: 26 - DNA, 21 bp, synthetic construct
  source 1..21: mol_type = other DNA
tactaaatgtgctcttaggct

SEQ ID NO: 27 - DNA, 21 bp, synthetic construct
  source 1..21: mol_type = other DNA
tactaaatgtgctgttaggct

SEQ ID NO: 28 - DNA, 21 bp, synthetic construct
  source 1..21: mol_type = other DNA
actaaatgtgctcttaggctt

SEQ ID NO: 29 - DNA, 21 bp, synthetic construct
  source 1..21: mol_type = other DNA
actaaatgtgctgttaggctt

SEQ ID NO: 30 - DNA, 21 bp, synthetic construct
  source 1..21: mol_type = other DNA
ctaaatgtgctcttaggctta

SEQ ID NO: 31 - DNA, 21 bp, synthetic construct
  source 1..21: mol_type = other DNA
ctaaatgtgctgttaggctta

SEQ ID NO: 32 - DNA, 21 bp, synthetic construct
  source 1..21: mol_type = other DNA
taaatgtgctcttaggcttac

SEQ ID NO: 33 - DNA, 21 bp, synthetic construct
  source 1..21: mol_type = other DNA
taaatgtgctgttaggcttac

SEQ ID NO: 34 - DNA, 21 bp, synthetic construct
  source 1..21: mol_type = other DNA
aaaagtttggagggtttcttc

SEQ ID NO: 35 - DNA, 21 bp, synthetic construct
  source 1..21: mol_type = other DNA
agtttggagggtttcttcgct

SEQ ID NO: 36 - DNA, 21 bp, synthetic construct
  source 1..21: mol_type = other DNA
gtttggagggtttctccgctc

SEQ ID NO: 37 - DNA, 21 bp, synthetic construct
  source 1..21: mol_type = other DNA
gtttggagggtttcttcgctc

SEQ ID NO: 38 - DNA, 21 bp, synthetic construct
  source 1..21: mol_type = other DNA
tttggagggtttctccgctca

SEQ ID NO: 39 - DNA, 21 bp, synthetic construct
  source 1..21: mol_type = other DNA
tttggagggtttcttcgctca

SEQ ID NO: 40 - DNA, 21 bp, synthetic construct
  source 1..21: mol_type = other DNA
ttggagggtttctccgctcag

SEQ ID NO: 41 - DNA, 21 bp, synthetic construct
  source 1..21: mol_type = other DNA
ttggagggtttcttcgctcag

SEQ ID NO: 42 - DNA, 21 bp, synthetic construct
  source 1..21: mol_type = other DNA
tggagggtttcttcgctcagc

SEQ ID NO: 43 - DNA, 21 bp, synthetic construct
  source 1..21: mol_type = other DNA
tggagggtttctccgctcagc

SEQ ID NO: 44 - DNA, 21 bp, synthetic construct
  source 1..21: mol_type = other DNA
agatggggacagtaattcaac

SEQ ID NO: 45 - DNA, 21 bp, synthetic construct
  source 1..21: mol_type = other DNA
agatggggacagtacttcaac

SEQ ID NO: 46 - DNA, 21 bp, synthetic construct
  source 1..21: mol_type = other DNA
gatggggacagtaattcaacg

SEQ ID NO: 47 - DNA, 21 bp, synthetic construct
  source 1..21: mol_type = other DNA
gatggggacagtacttcaacg

SEQ ID NO: 48 - DNA, 21 bp, synthetic construct
  source 1..21: mol_type = other DNA
atggggacagtacttcaacgc

SEQ ID NO: 49 - DNA, 21 bp, synthetic construct
  source 1..21: mol_type = other DNA
atggggacagtaattcaacgc

SEQ ID NO: 50 - DNA, 21 bp, synthetic construct
  source 1..21: mol_type = other DNA
tggggacagtacttcaacgct

SEQ ID NO: 51 - DNA, 21 bp, synthetic construct
  source 1..21: mol_type = other DNA
tggggacagtaattcaacgct

SEQ ID NO: 52 - DNA, 21 bp, synthetic construct
  source 1..21: mol_type = other DNA
ggggacagtaattcaacgcta

SEQ ID NO: 53 - DNA, 21 bp, synthetic construct
  source 1..21: mol_type = other DNA
ggggacagtacttcaacgcta

SEQ ID NO: 54 - DNA, 21 bp, synthetic construct
  source 1..21: mol_type = other DNA
cgagctgcctgcagagccggc

SEQ ID NO: 55 - DNA, 21 bp, synthetic construct
  source 1..21: mol_type = other DNA
gagctgcctgcagagccggcg

SEQ ID NO: 56 - DNA, 21 bp, synthetic construct
  source 1..21: mol_type = other DNA
agctgcctgcagagccggcgg

SEQ ID NO: 57 - DNA, 21 bp, synthetic construct
  source 1..21: mol_type = other DNA
gctgcctgcagagccggcggc

SEQ ID NO: 58 - DNA, 21 bp, synthetic construct
  source 1..21: mol_type = other DNA
tgcctgcagagccggcggcct

SEQ ID NO: 59 - DNA, 21 bp, synthetic construct
  source 1..21: mol_type = other DNA
ccacgcctgctccctcatcta

SEQ ID NO: 60 - DNA, 21 bp, synthetic construct
  source 1..21: mol_type = other DNA
cacgcctgctccctcatccac

SEQ ID NO: 61 - DNA, 21 bp, synthetic construct
  source 1..21: mol_type = other DNA
cacgcctgctccctcatctac

SEQ ID NO: 62 - DNA, 21 bp, synthetic construct
  source 1..21: mol_type = other DNA
acgcctgctccctcatccact

SEQ ID NO: 63 - DNA, 21 bp, synthetic construct
  source 1..21: mol_type = other DNA
cgcctgctccctcatccactg

SEQ ID NO: 64 - DNA, 21 bp, synthetic construct
  source 1..21: mol_type = other DNA
gctccctcatctactgtgtgc

SEQ ID NO: 65 - DNA, 21 bp, synthetic construct
  source 1..21: mol_type = other DNA
gctccctcatccactgtgtgc

SEQ ID NO: 66 - DNA, 21 bp, synthetic construct
  source 1..21: mol_type = other DNA
ctccctcatctactgtgtgca

SEQ ID NO: 67 - DNA, 21 bp, synthetic construct
  source 1..21: mol_type = other DNA
agcgcaaccagtttgagctga

SEQ ID NO: 68 - DNA, 21 bp, synthetic construct
  source 1..21: mol_type = other DNA
agcgcaaccagtttgagctaa

SEQ ID NO: 69 - DNA, 21 bp, synthetic construct
  source 1..21: mol_type = other DNA
gcgcaaccagtttgagctgat

SEQ ID NO: 70 - DNA, 21 bp, synthetic construct
  source 1..21: mol_type = other DNA
gcgcaaccagtttgagctaat

SEQ ID NO: 71 - DNA, 21 bp, synthetic construct
  source 1..21: mol_type = other DNA
cgcaaccagtttgagctaatg

SEQ ID NO: 72 - DNA, 21 bp, synthetic construct
  source 1..21: mol_type = other DNA
gcaaccagtttgagctaatgt

SEQ ID NO: 73 - DNA, 21 bp, synthetic construct
  source 1..21: mol_type = other DNA
aaccagtttgagctgatgtat

SEQ ID NO: 74 - DNA, 21 bp, synthetic construct
  source 1..21: mol_type = other DNA
accagtttgagctgatgtatg

SEQ ID NO: 75 - DNA, 21 bp, synthetic construct
  source 1..21: mol_type = other DNA
accagtttgagctaatgtatg

SEQ ID NO: 76 - DNA, 21 bp, synthetic construct
  source 1..21: mol_type = other DNA
ccagtttgagctgatgtatgt

SEQ ID NO: 77 - DNA, 21 bp, synthetic construct
  source 1..21: mol_type = other DNA
ccagtttgagctaatgtatgt

SEQ ID NO: 78 - DNA, 21 bp, synthetic construct
  source 1..21: mol_type = other DNA
cagtttgagctgatgtatgtg

SEQ ID NO: 79 - DNA, 21 bp, synthetic construct
  source 1..21: mol_type = other DNA
agtttgagctgatgtatgtga

SEQ ID NO: 80 - DNA, 21 bp, synthetic construct
  source 1..21: mol_type = other DNA
agtttgagctaatgtatgtga

SEQ ID NO: 81 - DNA, 21 bp, synthetic construct
  source 1..21: mol_type = other DNA
agaagctgctgctgcagatca

SEQ ID NO: 82 - DNA, 21 bp, synthetic construct
  source 1..21: mol_type = other DNA
agaagctgctgctacagatca

SEQ ID NO: 83 - DNA, 21 bp, synthetic construct
  source 1..21: mol_type = other DNA
gaagctgctgctgcagatcaa

SEQ ID NO: 84 - DNA, 21 bp, synthetic construct
  source 1..21: mol_type = other DNA
gaagctgctgctacagatcaa

SEQ ID NO: 85 - DNA, 21 bp, synthetic construct
  source 1..21: mol_type = other DNA
aagctgctgctgcagatcaac

SEQ ID NO: 86 - DNA, 21 bp, synthetic construct
  source 1..21: mol_type = other DNA
aagctgctgctacagatcaac

SEQ ID NO: 87 - DNA, 21 bp, synthetic construct
  source 1..21: mol_type = other DNA
agctgctgctacagatcaacc

SEQ ID NO: 88 - DNA, 21 bp, synthetic construct
  source 1..21: mol_type = other DNA
gaggaatgggacgaggaagag

SEQ ID NO: 89 - DNA, 21 bp, synthetic construct
  source 1..21: mol_type = other DNA
aggaagaggaggaggccgacg

SEQ ID NO: 90 - DNA, 21 bp, synthetic construct
  source 1..21: mol_type = other DNA
ggaagaggaggaggccgacgc

SEQ ID NO: 91 - DNA, 21 bp, synthetic construct
  source 1..21: mol_type = other DNA
gaagaggaggaggccgacgcc

SEQ ID NO: 92 - DNA, 21 bp, synthetic construct
  source 1..21: mol_type = other DNA
ggttggagccctgcacggcgt

SEQ ID NO: 93 - DNA, 21 bp, synthetic construct
  source 1..21: mol_type = other DNA
ggttggagccctgcacggcat

SEQ ID NO: 94 - DNA, 21 bp, synthetic construct
  source 1..21: mol_type = other DNA
gttggagccctgcacggcatc

SEQ ID NO: 95 - DNA, 21 bp, synthetic construct
  source 1..21: mol_type = other DNA
gttggagccctgcacggcgtc

SEQ ID NO: 96 - DNA, 21 bp, synthetic construct
  source 1..21: mol_type = other DNA
ttggagccctgcacggcatcc

SEQ ID NO: 97 - DNA, 21 bp, synthetic construct
  source 1..21: mol_type = other DNA
ttggagccctgcacggcgtcc

SEQ ID NO: 98 - DNA, 21 bp, synthetic construct
  source 1..21: mol_type = other DNA
tggagccctgcacggcgtcct

SEQ ID NO: 99 - DNA, 21 bp, synthetic construct
  source 1..21: mol_type = other DNA
ggagccctgcacggcgtcctc

SEQ ID NO: 100 - DNA, 21 bp, synthetic construct
  source 1..21: mol_type = other DNA
ggagccctgcacggcatcctc

SEQ ID NO: 101 - DNA, 21 bp, synthetic construct
  source 1..21: mol_type = other DNA
gagccctgcacggcatcctct

SEQ ID NO: 102 - DNA, 21 bp, synthetic construct
  source 1..21: mol_type = other DNA
gagccctgcacggcgtcctct

SEQ ID NO: 103 - DNA, 21 bp, synthetic construct
  source 1..21: mol_type = other DNA
agccctgcacggcgtcctcta

SEQ ID NO: 104 - DNA, 21 bp, synthetic construct
  source 1..21: mol_type = other DNA
agccctgcacggcatcctcta

SEQ ID NO: 105 - DNA, 21 bp, synthetic construct
  source 1..21: mol_type = other DNA
cctgcacggcgtcctctatgt

SEQ ID NO: 106 - DNA, 21 bp, synthetic construct
  source 1..21: mol_type = other DNA
cctgcacggcatcctctatgt

SEQ ID NO: 107 - DNA, 21 bp, synthetic construct
  source 1..21: mol_type = other DNA
ggagcctttggaagtctgtgc

SEQ ID NO: 108 - DNA, 21 bp, synthetic construct
  source 1..21: mol_type = other DNA
ggagcctttggaagtctgcgc

SEQ ID NO: 109 - DNA, 21 bp, synthetic construct
  source 1..21: mol_type = other DNA
gagcctttggaagtctgtgcc

SEQ ID NO: 110 - DNA, 21 bp, synthetic construct
  source 1..21: mol_type = other DNA
gagcctttggaagtctgcgcc

SEQ ID NO: 111 - DNA, 21 bp, synthetic construct
  source 1..21: mol_type = other DNA
agcctttggaagtctgtgccc

SEQ ID NO: 112 - DNA, 21 bp, synthetic construct
  source 1..21: mol_type = other DNA
agcctttggaagtctgcgccc

SEQ ID NO: 113 - DNA, 21 bp, synthetic construct
  source 1..21: mol_type = other DNA
gcctttggaagtctgcgccct

SEQ ID NO: 114 - DNA, 21 bp, synthetic construct
  source 1..21: mol_type = other DNA
cctttggaagtctgcgccctt

SEQ ID NO: 115 - DNA, 21 bp, synthetic construct
  source 1..21: mol_type = other DNA
cctttggaagtctgtgccctt

SEQ ID NO: 116 - DNA, 21 bp, synthetic construct
  source 1..21: mol_type = other DNA
ctttggaagtctgtgcccttg

SEQ ID NO: 117 - DNA, 21 bp, synthetic construct
  source 1..21: mol_type = other DNA
ctttggaagtctgcgcccttg

SEQ ID NO: 118 - DNA, 21 bp, synthetic construct
  source 1..21: mol_type = other DNA
tttggaagtctgtgcccttgt

SEQ ID NO: 119 - DNA, 21 bp, synthetic construct
  source 1..21: mol_type = other DNA
tttggaagtctgcgcccttgt

SEQ ID NO: 120 - DNA, 21 bp, synthetic construct
  source 1..21: mol_type = other DNA
ttggaagtctgtgcccttgtg

SEQ ID NO: 121 - DNA, 21 bp, synthetic construct
  source 1..21: mol_type = other DNA
ttggaagtctgcgcccttgtg

SEQ ID NO: 122 - DNA, 21 bp, synthetic construct
  source 1..21: mol_type = other DNA
tggaagtctgcgcccttgtgc

SEQ ID NO: 123 - DNA, 21 bp, synthetic construct
  source 1..21: mol_type = other DNA
tggaagtctgtgcccttgtgc

SEQ ID NO: 124 - DNA, 21 bp, synthetic construct
  source 1..21: mol_type = other DNA
gctggttgttgccaggttgca

SEQ ID NO: 125 - DNA, 21 bp, synthetic construct
  source 1..21: mol_type = other DNA
gctggttgttgccaggttaca

SEQ ID NO: 126 - DNA, 21 bp, synthetic construct
  source 1..21: mol_type = other DNA
ctggttgttgccaggttgcag

SEQ ID NO: 127 - DNA, 21 bp, synthetic construct
  source 1..21: mol_type = other DNA
ctggttgttgccaggttacag

SEQ ID NO: 128 - DNA, 21 bp, synthetic construct
  source 1..21: mol_type = other DNA
tggttgttgccaggttacagc

SEQ ID NO: 129 - DNA, 21 bp, synthetic construct
  source 1..21: mol_type = other DNA
tggttgttgccaggttgcagc

SEQ ID NO: 130 - DNA, 21 bp, synthetic construct
  source 1..21: mol_type = other DNA
ggttgttgccaggttgcagct

SEQ ID NO: 131 - DNA, 21 bp, synthetic construct
  source 1..21: mol_type = other DNA
ggttgttgccaggttacagct

SEQ ID NO: 132 - DNA, 21 bp, synthetic construct
  source 1..21: mol_type = other DNA
gttgttgccaggttacagctg

SEQ ID NO: 133 - DNA, 21 bp, synthetic construct
  source 1..21: mol_type = other DNA
tgttgccaggttacagctgct

SEQ ID NO: 134 - DNA, 21 bp, synthetic construct
  source 1..21: mol_type = other DNA
cctccctcctgcaggctggct

SEQ ID NO: 135 - DNA, 21 bp, synthetic construct
  source 1..21: mol_type = other DNA
cctccctcctgcaggctgggt

SEQ ID NO: 136 - DNA, 21 bp, synthetic construct
  source 1..21: mol_type = other DNA
ctccctcctgcaggctggctg

SEQ ID NO: 137 - DNA, 21 bp, synthetic construct
  source 1..21: mol_type = other DNA
ctccctcctgcaggctgggtg

SEQ ID NO: 138 - DNA, 21 bp, synthetic construct
  source 1..21: mol_type = other DNA
tccctcctgcaggctggctgt

SEQ ID NO: 139 - DNA, 21 bp, synthetic construct
  source 1..21: mol_type = other DNA
ccctcctgcaggctgggtgtt

SEQ ID NO: 140 - DNA, 21 bp, synthetic construct
  source 1..21: mol_type = other DNA
ccctcctgcaggctggctgtt

SEQ ID NO: 141 - DNA, 21 bp, synthetic construct
  source 1..21: mol_type = other DNA
cctcctgcaggctggctgttg

SEQ ID NO: 142 - DNA, 21 bp, synthetic construct
  source 1..21: mol_type = other DNA
cctcctgcaggctgggtgttg

SEQ ID NO: 143 - DNA, 21 bp, synthetic construct
  source 1..21: mol_type = other DNA
ctcctgcaggctggctgttgg

SEQ ID NO: 144 - DNA, 21 bp, synthetic construct
  source 1..21: mol_type = other DNA
ctcctgcaggctgggtgttgg

SEQ ID NO: 145 - DNA, 21 bp, synthetic construct
  source 1..21: mol_type = other DNA
tcctgcaggctggctgttggc

SEQ ID NO: 146 - DNA, 21 bp, synthetic construct
  source 1..21: mol_type = other DNA
cctgcaggctggctgttggcc

SEQ ID NO: 147 - DNA, 21 bp, synthetic construct
  source 1..21: mol_type = other DNA
tgcaggctgggtgttggcccc

SEQ ID NO: 148 - DNA, 21 bp, synthetic construct
  source 1..21: mol_type = other DNA
tgcaggctggctgttggcccc

SEQ ID NO: 149 - DNA, 21 bp, synthetic construct
  source 1..21: mol_type = other DNA
aggctgggtgttggcccctgt

SEQ ID NO: 150 - DNA, 21 bp, synthetic construct
  source 1..21: mol_type = other DNA
ggctggctgttggcccctgtg

SEQ ID NO: 151 - DNA, 21 bp, synthetic construct
  source 1..21: mol_type = other DNA
ggctgggtgttggcccctgtg

SEQ ID NO: 152 - DNA, 21 bp, synthetic construct
  source 1..21: mol_type = other DNA
gctgggtgttggcccctgtgc

SEQ ID NO: 153 - DNA, 21 bp, synthetic construct
  source 1..21: mol_type = other DNA
gctggctgttggcccctctgc

SEQ ID NO: 154 - DNA, 21 bp, synthetic construct
  source 1..21: mol_type = other DNA
gctggctgttggcccctgtgc

SEQ ID NO: 155 - DNA, 21 bp, synthetic construct
  source 1..21: mol_type = other DNA
gctgggtgttggcccctctgc

SEQ ID NO: 156 - DNA, 21 bp, synthetic construct
  source 1..21: mol_type = other DNA
ctggctgttggcccctgtgct

SEQ ID NO: 157 - DNA, 21 bp, synthetic construct
  source 1..21: mol_type = other DNA
ctgggtgttggcccctgtgct

SEQ ID NO: 158 - DNA, 21 bp, synthetic construct
  source 1..21: mol_type = other DNA
tggctgttggcccctgtgctg

SEQ ID NO: 159 - DNA, 21 bp, synthetic construct
  source 1..21: mol_type = other DNA
tgggtgttggcccctgtgctg

SEQ ID NO: 160 - DNA, 21 bp, synthetic construct
  source 1..21: mol_type = other DNA
ctgttggcccctctgctgtcc

SEQ ID NO: 161 - DNA, 21 bp, synthetic construct
  source 1..21: mol_type = other DNA
gtgttggcccctctgctgtcc

SEQ ID NO: 162 - DNA, 21 bp, synthetic construct
  source 1..21: mol_type = other DNA
ctgttggcccctgtgctgtcc

SEQ ID NO: 163 - DNA, 21 bp, synthetic construct
  source 1..21: mol_type = other DNA
gtgttggcccctgtgctgtcc

SEQ ID NO: 164 - DNA, 21 bp, synthetic construct
  source 1..21: mol_type = other DNA
tgttggcccctctgctgtcct

SEQ ID NO: 165 - DNA, 21 bp, synthetic construct
  source 1..21: mol_type = other DNA
tgttggcccctgtgctgtcct

SEQ ID NO: 166 - DNA, 21 bp, synthetic construct
  source 1..21: mol_type = other DNA
gttggcccctgtgctgtcctg

SEQ ID NO: 167 - DNA, 21 bp, synthetic construct
  source 1..21: mol_type = other DNA
gttggcccctctgctgtcctg

SEQ ID NO: 168 - DNA, 21 bp, synthetic construct
  source 1..21: mol_type = other DNA
gatgcacagatgccatggcat

SEQ ID NO: 169 - DNA, 21 bp, synthetic construct
  source 1..21: mol_type = other DNA
atgcacagatgccatggcctg

SEQ ID NO: 170 - DNA, 21 bp, synthetic construct
  source 1..21: mol_type = other DNA
atgcacagatgccatggcatg

SEQ ID NO: 171 - DNA, 21 bp, synthetic construct
  source 1..21: mol_type = other DNA
tgcacagatgccatggcctgt

SEQ ID NO: 172 - DNA, 21 bp, synthetic construct
  source 1..21: mol_type = other DNA
tgcacagatgccatggcatgt

SEQ ID NO: 173 - DNA, 21 bp, synthetic construct
  source 1..21: mol_type = other DNA
gcacagatgccatggcctgtg

SEQ ID NO: 174 - DNA, 21 bp, synthetic construct
  source 1..21: mol_type = other DNA
gcacagatgccatggcatgtg

SEQ ID NO: 175 - DNA, 21 bp, synthetic construct
  source 1..21: mol_type = other DNA
cacagatgccatggcctgtgc

SEQ ID NO: 176 - DNA, 21 bp, synthetic construct
  source 1..21: mol_type = other DNA
cacagatgccatggcatgtgc

SEQ ID NO: 177 - DNA, 21 bp, synthetic construct
  source 1..21: mol_type = other DNA
acagatgccatggcatgtgct

SEQ ID NO: 178 - DNA, 21 bp, synthetic construct
  source 1..21: mol_type = other DNA
cagatgccatggcctgtgctg

SEQ ID NO: 179 - DNA, 21 bp, synthetic construct
  source 1..21: mol_type = other DNA
cagatgccatggcatgtgctg

SEQ ID NO: 180 - DNA, 21 bp, synthetic construct
  source 1..21: mol_type = other DNA
gctgggggtgctagacacccg

SEQ ID NO: 181 - DNA, 21 bp, synthetic construct
  source 1..21: mol_type = other DNA
gctgggggtgctagacacctg

SEQ ID NO: 182 - DNA, 21 bp, synthetic construct
  source 1..21: mol_type = other DNA
ctgggggtgctagacacccgg

SEQ ID NO: 183 - DNA, 21 bp, synthetic construct
  source 1..21: mol_type = other DNA
tgggggtgctagacacccggc

SEQ ID NO: 184 - DNA, 21 bp, synthetic construct
  source 1..21: mol_type = other DNA
gggggtgctagacacccggca

SEQ ID NO: 185 - DNA, 21 bp, synthetic construct
  source 1..21: mol_type = other DNA
gggggtgctagacacctggca

SEQ ID NO: 186 - DNA, 21 bp, synthetic construct
  source 1..21: mol_type = other DNA
ggggtgctagacacccggcac

SEQ ID NO: 187 - DNA, 21 bp, synthetic construct
  source 1..21: mol_type = other DNA
ggggtgctagacacctggcac

SEQ ID NO: 188 - DNA, 21 bp, synthetic construct
  source 1..21: mol_type = other DNA
gggtgctagacacctggcacc

SEQ ID NO: 189 - DNA, 21 bp, synthetic construct
  source 1..21: mol_type = other DNA
gggtgctagacacccggcacc

SEQ ID NO: 190 - DNA, 21 bp, synthetic construct
  source 1..21: mol_type = other DNA
ggtgctagacacccggcacca

SEQ ID NO: 191 - DNA, 21 bp, synthetic construct
  source 1..21: mol_type = other DNA
ggtgctagacacctggcacca

SEQ ID NO: 192 - DNA, 21 bp, synthetic construct
  source 1..21: mol_type = other DNA
gtgctagacacccggcaccat

SEQ ID NO: 193 - DNA, 21 bp, synthetic construct
  source 1..21: mol_type = other DNA
gtgctagacacctggcaccat

SEQ ID NO: 194 - DNA, 21 bp, synthetic construct
  source 1..21: mol_type = other DNA
tgctagacacctggcaccatt

SEQ ID NO: 195 - DNA, 21 bp, synthetic construct
  source 1..21: mol_type = other DNA
tgctagacacccggcaccatt

SEQ ID NO: 196 - DNA, 21 bp, synthetic construct
  source 1..21: mol_type = other DNA
gctagacacctggcaccattc

SEQ ID NO: 197 - DNA, 21 bp, synthetic construct
  source 1..21: mol_type = other DNA
gctagacacccggcaccattc

SEQ ID NO: 198 - DNA, 21 bp, synthetic construct
  source 1..21: mol_type = other DNA
atttgggagctctgcttgctg

SEQ ID NO: 199 - DNA, 21 bp, synthetic construct
  source 1..21: mol_type = other DNA
ttgggagctctgcttgccgac

SEQ ID NO: 200 - DNA, 21 bp, synthetic construct
  source 1..21: mol_type = other DNA
ttgggagctctgcttgctgac

SEQ ID NO: 201 - DNA, 21 bp, synthetic construct
  source 1..21: mol_type = other DNA
tgggagctctgcttgctgact

SEQ ID NO: 202 - DNA, 21 bp, synthetic construct
  source 1..21: mol_type = other DNA
tgggagctctgcttgccgact

SEQ ID NO: 203 - DNA, 21 bp, synthetic construct
  source 1..21: mol_type = other DNA
gggagctctgcttgccgactg

SEQ ID NO: 204 - DNA, 21 bp, synthetic construct
  source 1..21: mol_type = other DNA
gggagctctgcttgctgactg

SEQ ID NO: 205 - DNA, 21 bp, synthetic construct
  source 1..21: mol_type = other DNA
ggagctctgcttgctgactgg

SEQ ID NO: 206 - DNA, 21 bp, synthetic construct
  source 1..21: mol_type = other DNA
ggagctctgcttgccgactgg

SEQ ID NO: 207 - DNA, 21 bp, synthetic construct
  source 1..21: mol_type = other DNA
gagctctgcttgctgactggc

SEQ ID NO: 208 - DNA, 21 bp, synthetic construct
  source 1..21: mol_type = other DNA
gagctctgcttgccgactggc

SEQ ID NO: 209 - DNA, 21 bp, synthetic construct
  source 1..21: mol_type = other DNA
agctctgcttgctgactggct

SEQ ID NO: 210 - DNA, 21 bp, synthetic construct
  source 1..21: mol_type = other DNA
agctctgcttgccgactggct

SEQ ID NO: 211 - DNA, 21 bp, synthetic construct
  source 1..21: mol_type = other DNA
gctctgcttgctgactggctg

SEQ ID NO: 212 - DNA, 21 bp, synthetic construct
  source 1..21: mol_type = other DNA
gctctgcttgccgactggctg

SEQ ID NO: 213 - DNA, 21 bp, synthetic construct
  source 1..21: mol_type = other DNA
ctctgcttgctgactggctgt

SEQ ID NO: 214 - DNA, 21 bp, synthetic construct
  source 1..21: mol_type = other DNA
ctctgcttgccgactggctgt

SEQ ID NO: 215 - DNA, 21 bp, synthetic construct
  source 1..21: mol_type = other DNA
ccaggcaaggttggcgactat

SEQ ID NO: 216 - DNA, 21 bp, synthetic construct
  source 1..21: mol_type = other DNA
ccaggcaaggttggcgactgt

SEQ ID NO: 217 - DNA, 21 bp, synthetic construct
  source 1..21: mol_type = other DNA
caggcaaggttggcgactgtc

SEQ ID NO: 218 - DNA, 21 bp, synthetic construct
  source 1..21: mol_type = other DNA
caggcaaggttggcgactatc

SEQ ID NO: 219 - DNA, 21 bp, synthetic construct
  source 1..21: mol_type = other DNA
aggcaaggttggcgactgtca

SEQ ID NO: 220 - DNA, 21 bp, synthetic construct
  source 1..21: mol_type = other DNA
aggcaaggttggcgactatca

SEQ ID NO: 221 - DNA, 21 bp, synthetic construct
  source 1..21: mol_type = other DNA
ggcaaggttggcgactgtcat

SEQ ID NO: 222 - DNA, 21 bp, synthetic construct
  source 1..21: mol_type = other DNA
ggcaaggttggcgactatcat

SEQ ID NO: 223 - DNA, 21 bp, synthetic construct
  source 1..21: mol_type = other DNA
gcaaggttggcgactatcatg

SEQ ID NO: 224 - DNA, 21 bp, synthetic construct
  source 1..21: mol_type = other DNA
caaggttggcgactatcatgt

SEQ ID NO: 225 - DNA, 21 bp, synthetic construct
  source 1..21: mol_type = other DNA
aaggttggcgactgtcatgtg

SEQ ID NO: 226 - DNA, 21 bp, synthetic construct
  source 1..21: mol_type = other DNA
aaggttggcgactatcatgtg

SEQ ID NO: 227 - DNA, 21 bp, synthetic construct
  source 1..21: mol_type = other DNA
aggttggcgactgtcatgtgg

SEQ ID NO: 228 - DNA, 21 bp, synthetic construct
  source 1..21: mol_type = other DNA
aggttggcgactatcatgtgg

SEQ ID NO: 229 - DNA, 21 bp, synthetic construct
  source 1..21: mol_type = other DNA
ggttggcgactgtcatgtggc

SEQ ID NO: 230 - DNA, 21 bp, synthetic construct
  source 1..21: mol_type = other DNA
ggttggcgactatcatgtggc

SEQ ID NO: 231 - DNA, 21 bp, synthetic construct
  source 1..21: mol_type = other DNA
gttggcgactgtcatgtggct

SEQ ID NO: 232 - DNA, 21 bp, synthetic construct
  source 1..21: mol_type = other DNA
gttggcgactatcatgtggct

SEQ ID NO: 233 - DNA, 21 bp, synthetic construct
  source 1..21: mol_type = other DNA
cccccgcttcctccctctgtg

SEQ ID NO: 234 - DNA, 21 bp, synthetic construct
  source 1..21: mol_type = other DNA
cccgcttcctccctctgtggg

SEQ ID NO: 235 - DNA, 21 bp, synthetic construct
  source 1..21: mol_type = other DNA
cccgcttcctccctctgcggg

SEQ ID NO: 236 - DNA, 21 bp, synthetic construct
  source 1..21: mol_type = other DNA
ccgcttcctccctctgcgggg

SEQ ID NO: 237 - DNA, 21 bp, synthetic construct
  source 1..21: mol_type = other DNA
cgcttcctccctctgtgggga

SEQ ID NO: 238 - DNA, 21 bp, synthetic construct
  source 1..21: mol_type = other DNA
gcttcctccctctgcggggag

SEQ ID NO: 239 - DNA, 21 bp, synthetic construct
  source 1..21: mol_type = other DNA
gcttcctccctctgtggggag

SEQ ID NO: 240 - DNA, 21 bp, synthetic construct
  source 1..21: mol_type = other DNA
cttcctccctctgcggggagg

SEQ ID NO: 241 - DNA, 21 bp, synthetic construct
  source 1..21: mol_type = other DNA
cttcctccctctgtggggagg

SEQ ID NO: 242 - DNA, 21 bp, synthetic construct
  source 1..21: mol_type = other DNA
ttcctccctctgcggggagga

SEQ ID NO: 243 - DNA, 21 bp, synthetic construct
  source 1..21: mol_type = other DNA
ttcctccctctgtggggagga

SEQ ID NO: 244 - DNA, 21 bp, synthetic construct
  source 1..21: mol_type = other DNA
tcctccctctgcggggaggac

SEQ ID NO: 245 - DNA, 21 bp, synthetic construct
  source 1..21: mol_type = other DNA
cctccctctgcggggaggacc

SEQ ID NO: 246 - RNA, 21 nt, synthetic construct
  source 1..21: mol_type = other RNA
agcagaacctgagcggccgtc

SEQ ID NO: 247 - RNA, 21 nt, synthetic construct
  source 1..21: mol_type = other RNA
aacagaacctgagcggccgtc

SEQ ID NO: 248 - RNA, 21 nt, synthetic construct
  source 1..21: mol_type = other RNA
aagcagaacctgagcggccgt

SEQ ID NO: 249 - RNA, 21 nt, synthetic construct
  source 1..21: mol_type = other RNA
aaacagaacctgagcggccgt

SEQ ID NO: 250 - RNA, 21 nt, synthetic construct
  source 1..21: mol_type = other RNA
aaagcagaacctgagcggccg

SEQ ID NO: 251 - RNA, 21 nt, synthetic construct
  source 1..21: mol_type = other RNA
aaaacagaacctgagcggccg

SEQ ID NO: 252 - RNA, 21 nt, synthetic construct
  source 1..21: mol_type = other RNA
aaaagcagaacctgagcggcc

SEQ ID NO: 253 - RNA, 21 nt, synthetic construct
  source 1..21: mol_type = other RNA
aaaaacagaacctgagcggcc

SEQ ID NO: 254 - RNA, 21 nt, synthetic construct
  source 1..21: mol_type = other RNA
taaaagcagaacctgagcggc

SEQ ID NO: 255 - RNA, 21 nt, synthetic construct
  source 1..21: mol_type = other RNA
taaaaacagaacctgagcggc

SEQ ID NO: 256 - RNA, 21 nt, synthetic construct
  source 1..21: mol_type = other RNA
gtaaaagcagaacctgagcgg

SEQ ID NO: 257 - RNA, 21 nt, synthetic construct
  source 1..21: mol_type = other RNA
gtaaaaacagaacctgagcgg

SEQ ID NO: 258 - RNA, 21 nt, synthetic construct
  source 1..21: mol_type = other RNA
ggtaaaagcagaacctgagcg

SEQ ID NO: 259 - RNA, 21 nt, synthetic construct
  source 1..21: mol_type = other RNA
ggtaaaaacagaacctgagcg

SEQ ID NO: 260 - RNA, 21 nt, synthetic construct
  source 1..21: mol_type = other RNA
aggtaaaagcagaacctgagc

SEQ ID NO: 261 - RNA, 21 nt, synthetic construct
  source 1..21: mol_type = other RNA
aggtaaaaacagaacctgagc

SEQ ID NO: 262 - RNA, 21 nt, synthetic construct
  source 1..21: mol_type = other RNA
caggtaaaagcagaacctgag

SEQ ID NO: 263 - RNA, 21 nt, synthetic construct
  source 1..21: mol_type = other RNA
caggtaaaaacagaacctgag

SEQ ID NO: 264 - RNA, 21 nt, synthetic construct
  source 1..21: mol_type = other RNA
taagagcacatttagtagcca

SEQ ID NO: 265 - RNA, 21 nt, synthetic construct
  source 1..21: mol_type = other RNA
taacagcacatttagtagcca

SEQ ID NO: 266 - RNA, 21 nt, synthetic construct
  source 1..21: mol_type = other RNA
ctaacagcacatttagtagcc

SEQ ID NO: 267 - RNA, 21 nt, synthetic construct
  source 1..21: mol_type = other RNA
cctaagagcacatttagtagc

SEQ ID NO: 268 - RNA, 21 nt, synthetic construct
  source 1..21: mol_type = other RNA
cctaacagcacatttagtagc

SEQ ID NO: 269 - RNA, 21 nt, synthetic construct
  source 1..21: mol_type = other RNA
gcctaagagcacatttagtag

SEQ ID NO: 270 - RNA, 21 nt, synthetic construct
  source 1..21: mol_type = other RNA
gcctaacagcacatttagtag

SEQ ID NO: 271 - RNA, 21 nt, synthetic construct
  source 1..21: mol_type = other RNA
agcctaagagcacatttagta

SEQ ID NO: 272 - RNA, 21 nt, synthetic construct
  source 1..21: mol_type = other RNA
agcctaacagcacatttagta

SEQ ID NO: 273 - RNA, 21 nt, synthetic construct
  source 1..21: mol_type = other RNA
aagcctaagagcacatttagt

SEQ ID NO: 274 - RNA, 21 nt, synthetic construct
  source 1..21: mol_type = other RNA
aagcctaacagcacatttagt

SEQ ID NO: 275 - RNA, 21 nt, synthetic construct
  source 1..21: mol_type = other RNA
taagcctaagagcacatttag

SEQ ID NO: 276 - RNA, 21 nt, synthetic construct
  source 1..21: mol_type = other RNA
taagcctaacagcacatttag

SEQ ID NO: 277 - RNA, 21 nt, synthetic construct
  source 1..21: mol_type = other RNA
gtaagcctaagagcacattta

SEQ ID NO: 278 - RNA, 21 nt, synthetic construct
  source 1..21: mol_type = other RNA
gtaagcctaacagcacattta

SEQ ID NO: 279 - RNA, 21 nt, synthetic construct
  source 1..21: mol_type = other RNA
gaagaaaccctccaaactttt

SEQ ID NO: 280 - RNA, 21 nt, synthetic construct
  source 1..21: mol_type = other RNA
agcgaagaaaccctccaaact

SEQ ID NO: 281 - RNA, 21 nt, synthetic construct
  source 1..21: mol_type = other RNA
gagcggagaaaccctccaaac

SEQ ID NO: 282 - RNA, 21 nt, synthetic construct
  source 1..21: mol_type = other RNA
gagcgaagaaaccctccaaac

SEQ ID NO: 283 - RNA, 21 nt, synthetic construct
  source 1..21: mol_type = other RNA
tgagcggagaaaccctccaaa

SEQ ID NO: 284 - RNA, 21 nt, synthetic construct
  source 1..21: mol_type = other RNA
tgagcgaagaaaccctccaaa

SEQ ID NO: 285 - RNA, 21 nt, synthetic construct
  source 1..21: mol_type = other RNA
ctgagcggagaaaccctccaa

SEQ ID NO: 286 - RNA, 21 nt, synthetic construct
  source 1..21: mol_type = other RNA
ctgagcgaagaaaccctccaa

SEQ ID NO: 287 - RNA, 21 nt, synthetic construct
  source 1..21: mol_type = other RNA
gctgagcgaagaaaccctcca

SEQ ID NO: 288 - RNA, 21 nt, synthetic construct
  source 1..21: mol_type = other RNA
gctgagcggagaaaccctcca

SEQ ID NO: 289 - RNA, 21 nt, synthetic construct
  source 1..21: mol_type = other RNA
gttgaattactgtccccatct

SEQ ID NO: 290 - RNA, 21 nt, synthetic construct
  source 1..21: mol_type = other RNA
gttgaagtactgtccccatct

SEQ ID NO: 291 - RNA, 21 nt, synthetic construct
  source 1..21: mol_type = other RNA
cgttgaattactgtccccatc

SEQ ID NO: 292 - RNA, 21 nt, synthetic construct
  source 1..21: mol_type = other RNA
cgttgaagtactgtccccatc

SEQ ID NO: 293 - RNA, 21 nt, synthetic construct
  source 1..21: mol_type = other RNA
gcgttgaagtactgtccccat

SEQ ID NO: 294 - RNA, 21 nt, synthetic construct
  source 1..21: mol_type = other RNA
gcgttgaattactgtccccat

SEQ ID NO: 295 - RNA, 21 nt, synthetic construct
  source 1..21: mol_type = other RNA
agcgttgaagtactgtcccca

SEQ ID NO: 296 - RNA, 21 nt, synthetic construct
  source 1..21: mol_type = other RNA
agcgttgaattactgtcccca

SEQ ID NO: 297 - RNA, 21 nt, synthetic construct
  source 1..21: mol_type = other RNA
tagcgttgaattactgtcccc

SEQ ID NO: 298 - RNA, 21 nt, synthetic construct
  source 1..21: mol_type = other RNA
tagcgttgaagtactgtcccc

SEQ ID NO: 299 - RNA, 21 nt, synthetic construct
  source 1..21: mol_type = other RNA
gccggctctgcaggcagctcg

SEQ ID NO: 300 - RNA, 21 nt, synthetic construct
  source 1..21: mol_type = other RNA
cgccggctctgcaggcagctc

SEQ ID NO: 301 - RNA, 21 nt, synthetic construct
  source 1..21: mol_type = other RNA
ccgccggctctgcaggcagct

SEQ ID NO: 302 - RNA, 21 nt, synthetic construct
  source 1..21: mol_type = other RNA
gccgccggctctgcaggcagc

SEQ ID NO: 303 - RNA, 21 nt, synthetic construct
  source 1..21: mol_type = other RNA
aggccgccggctctgcaggca

SEQ ID NO: 304 - RNA, 21 nt, synthetic construct
  source 1..21: mol_type = other RNA
tagatgagggagcaggcgtgg

SEQ ID NO: 305 - RNA, 21 nt, synthetic construct
  source 1..21: mol_type = other RNA
gtggatgagggagcaggcgtg

SEQ ID NO: 306 - RNA, 21 nt, synthetic construct
  source 1..21: mol_type = other RNA
gtagatgagggagcaggcgtg

SEQ ID NO: 307 - RNA, 21 nt, synthetic construct
  source 1..21: mol_type = other RNA
agtggatgagggagcaggcgt

SEQ ID NO: 308 - RNA, 21 nt, synthetic construct
  source 1..21: mol_type = other RNA
cagtggatgagggagcaggcg

SEQ ID NO: 309 - RNA, 21 nt, synthetic construct
  source 1..21: mol_type = other RNA
gcacacagtagatgagggagc

SEQ ID NO: 310 - RNA, 21 nt, synthetic construct
  source 1..21: mol_type = other RNA
gcacacagtggatgagggagc

SEQ ID NO: 311 - RNA, 21 nt, synthetic construct
  source 1..21: mol_type = other RNA
tgcacacagtagatgagggag

SEQ ID NO: 312 - RNA, 21 nt, synthetic construct
  source 1..21: mol_type = other RNA
tcagctcaaactggttgcgct

SEQ ID NO: 313 - RNA, 21 nt, synthetic construct
  source 1..21: mol_type = other RNA
ttagctcaaactggttgcgct

SEQ ID NO: 314 - RNA, 21 nt, synthetic construct
  source 1..21: mol_type = other RNA
atcagctcaaactggttgcgc

SEQ ID NO: 315 - RNA, 21 nt, synthetic construct
  source 1..21: mol_type = other RNA
attagctcaaactggttgcgc

SEQ ID NO: 316 - RNA, 21 nt, synthetic construct
  source 1..21: mol_type = other RNA
cattagctcaaactggttgcg

SEQ ID NO: 317 - RNA, 21 nt, synthetic construct
  source 1..21: mol_type = other RNA
acattagctcaaactggttgc

SEQ ID NO: 318 - RNA, 21 nt, synthetic construct
  source 1..21: mol_type = other RNA
atacatcagctcaaactggtt

SEQ ID NO: 319 - RNA, 21 nt, synthetic construct
  source 1..21: mol_type = other RNA
catacatcagctcaaactggt

SEQ ID NO: 320 - RNA, 21 nt, synthetic construct
  source 1..21: mol_type = other RNA
catacattagctcaaactggt

SEQ ID NO: 321 - RNA, 21 nt, synthetic construct
  source 1..21: mol_type = other RNA
acatacatcagctcaaactgg

SEQ ID NO: 322 - RNA, 21 nt, synthetic construct
  source 1..21: mol_type = other RNA
acatacattagctcaaactgg

SEQ ID NO: 323 - RNA, 21 nt, synthetic construct
  source 1..21: mol_type = other RNA
cacatacatcagctcaaactg

SEQ ID NO: 324 - RNA, 21 nt, synthetic construct
  source 1..21: mol_type = other RNA
tcacatacatcagctcaaact

SEQ ID NO: 325 - RNA, 21 nt, synthetic construct
  source 1..21: mol_type = other RNA
tcacatacattagctcaaact

SEQ ID NO: 326 - RNA, 21 nt, synthetic construct
  source 1..21: mol_type = other RNA
tgatctgcagcagcagcttct

SEQ ID NO: 327 - RNA, 21 nt, synthetic construct
  source 1..21: mol_type = other RNA
tgatctgtagcagcagcttct

SEQ ID NO: 328 - RNA, 21 nt, synthetic construct
  source 1..21: mol_type = other RNA
ttgatctgcagcagcagcttc

SEQ ID NO: 329 - RNA, 21 nt, synthetic construct
  source 1..21: mol_type = other RNA
ttgatctgtagcagcagcttc

SEQ ID NO: 330 - RNA, 21 nt, synthetic construct
  source 1..21: mol_type = other RNA
gttgatctgcagcagcagctt

SEQ ID NO: 331 - RNA, 21 nt, synthetic construct
  source 1..21: mol_type = other RNA
gttgatctgtagcagcagctt

SEQ ID NO: 332 - RNA, 21 nt, synthetic construct
  source 1..21: mol_type = other RNA
ggttgatctgtagcagcagct

SEQ ID NO: 333 - RNA, 21 nt, synthetic construct
  source 1..21: mol_type = other RNA
ctcttcctcgtcccattcctc

SEQ ID NO: 334 - RNA, 21 nt, synthetic construct
  source 1..21: mol_type = other RNA
cgtcggcctcctcctcttcct

SEQ ID NO: 335 - RNA, 21 nt, synthetic construct
  source 1..21: mol_type = other RNA
gcgtcggcctcctcctcttcc

SEQ ID NO: 336 - RNA, 21 nt, synthetic construct
  source 1..21: mol_type = other RNA
ggcgtcggcctcctcctcttc

SEQ ID NO: 337 - RNA, 21 nt, synthetic construct
  source 1..21: mol_type = other RNA
acgccgtgcagggctccaacc

SEQ ID NO: 338 - RNA, 21 nt, synthetic construct
  source 1..21: mol_type = other RNA
atgccgtgcagggctccaacc

SEQ ID NO: 339 - RNA, 21 nt, synthetic construct
  source 1..21: mol_type = other RNA
gatgccgtgcagggctccaac

SEQ ID NO: 340 - RNA, 21 nt, synthetic construct
  source 1..21: mol_type = other RNA
gacgccgtgcagggctccaac

SEQ ID NO: 341 - RNA, 21 nt, synthetic construct
  source 1..21: mol_type = other RNA
ggatgccgtgcagggctccaa

SEQ ID NO: 342 - RNA, 21 nt, synthetic construct
  source 1..21: mol_type = other RNA
ggacgccgtgcagggctccaa

SEQ ID NO: 343 - RNA, 21 nt, synthetic construct
  source 1..21: mol_type = other RNA
aggacgccgtgcagggctcca

SEQ ID NO: 344 - RNA, 21 nt, synthetic construct
  source 1..21: mol_type = other RNA
gaggacgccgtgcagggctcc

SEQ ID NO: 345 - RNA, 21 nt, synthetic construct
  source 1..21: mol_type = other RNA
gaggatgccgtgcagggctcc

SEQ ID NO: 346 - RNA, 21 nt, synthetic construct
  source 1..21: mol_type = other RNA
agaggatgccgtgcagggctc

SEQ ID NO: 347 - RNA, 21 nt, synthetic construct
  source 1..21: mol_type = other RNA
agaggacgccgtgcagggctc

SEQ ID NO: 348 - RNA, 21 nt, synthetic construct
  source 1..21: mol_type = other RNA
tagaggacgccgtgcagggct

SEQ ID NO: 349 - RNA, 21 nt, synthetic construct
  source 1..21: mol_type = other RNA
tagaggatgccgtgcagggct

SEQ ID NO: 350 - RNA, 21 nt, synthetic construct
  source 1..21: mol_type = other RNA
acatagaggacgccgtgcagg

SEQ ID NO: 351 - RNA, 21 nt, synthetic construct
  source 1..21: mol_type = other RNA
acatagaggatgccgtgcagg

SEQ ID NO: 352 - RNA, 21 nt, synthetic construct
  source 1..21: mol_type = other RNA
gcacagacttccaaaggctcc

SEQ ID NO: 353 - RNA, 21 nt, synthetic construct
  source 1..21: mol_type = other RNA
gcgcagacttccaaaggctcc

SEQ ID NO: 354 - RNA, 21 nt, synthetic construct
  source 1..21: mol_type = other RNA
ggcacagacttccaaaggctc

SEQ ID NO: 355 - RNA, 21 nt, synthetic construct
  source 1..21: mol_type = other RNA
ggcgcagacttccaaaggctc

SEQ ID NO: 356 - RNA, 21 nt, synthetic construct
  source 1..21: mol_type = other RNA
gggcacagacttccaaaggct

SEQ ID NO: 357 - RNA, 21 nt, synthetic construct
  source 1..21: mol_type = other RNA
gggcgcagacttccaaaggct

SEQ ID NO: 358 - RNA, 21 nt, synthetic construct
  source 1..21: mol_type = other RNA
agggcgcagacttccaaaggc

SEQ ID NO: 359 - RNA, 21 nt, synthetic construct
  source 1..21: mol_type = other RNA
aagggcgcagacttccaaagg

SEQ ID NO: 360 - RNA, 21 nt, synthetic construct
  source 1..21: mol_type = other RNA
aagggcacagacttccaaagg

SEQ ID NO: 361 - RNA, 21 nt, synthetic construct
  source 1..21: mol_type = other RNA
caagggcacagacttccaaag

SEQ ID NO: 362 - RNA, 21 nt, synthetic construct
  source 1..21: mol_type = other RNA
caagggcgcagacttccaaag

SEQ ID NO: 363 - RNA, 21 nt, synthetic construct
  source 1..21: mol_type = other RNA
acaagggcacagacttccaaa

SEQ ID NO: 364 - RNA, 21 nt, synthetic construct
  source 1..21: mol_type = other RNA
acaagggcgcagacttccaaa

SEQ ID NO: 365 - RNA, 21 nt, synthetic construct
  source 1..21: mol_type = other RNA
cacaagggcacagacttccaa

SEQ ID NO: 366 - RNA, 21 nt, synthetic construct
  source 1..21: mol_type = other RNA
cacaagggcgcagacttccaa

SEQ ID NO: 367 - RNA, 21 nt, synthetic construct
  source 1..21: mol_type = other RNA
gcacaagggcgcagacttcca

SEQ ID NO: 368 - RNA, 21 nt, synthetic construct
  source 1..21: mol_type = other RNA
gcacaagggcacagacttcca

SEQ ID NO: 369 - RNA, 21 nt, synthetic construct
  source 1..21: mol_type = other RNA
tgcaacctggcaacaaccagc

SEQ ID NO: 370 - RNA, 21 nt, synthetic construct
  source 1..21: mol_type = other RNA
tgtaacctggcaacaaccagc

SEQ ID NO: 371 - RNA, 21 nt, synthetic construct
  source 1..21: mol_type = other RNA
ctgcaacctggcaacaaccag

SEQ ID NO: 372 - RNA, 21 nt, synthetic construct
  source 1..21: mol_type = other RNA
ctgtaacctggcaacaaccag

SEQ ID NO: 373 - RNA, 21 nt, synthetic construct
  source 1..21: mol_type = other RNA
gctgtaacctggcaacaacca

SEQ ID NO: 374 - RNA, 21 nt, synthetic construct
  source 1..21: mol_type = other RNA
gctgcaacctggcaacaacca

SEQ ID NO: 375 - RNA, 21 nt, synthetic construct
  source 1..21: mol_type = other RNA
agctgcaacctggcaacaacc

SEQ ID NO: 376 - RNA, 21 nt, synthetic construct
  source 1..21: mol_type = other RNA
agctgtaacctggcaacaacc

SEQ ID NO: 377 - RNA, 21 nt, synthetic construct
  source 1..21: mol_type = other RNA
cagctgtaacctggcaacaac

SEQ ID NO: 378 - RNA, 21 nt, synthetic construct
  source 1..21: mol_type = other RNA
agcagctgtaacctggcaaca

SEQ ID NO: 379 - RNA, 21 nt, synthetic construct
  source 1..21: mol_type = other RNA
agccagcctgcaggagggagg

SEQ ID NO: 380 - RNA, 21 nt, synthetic construct
  source 1..21: mol_type = other RNA
acccagcctgcaggagggagg

SEQ ID NO: 381 - RNA, 21 nt, synthetic construct
  source 1..21: mol_type = other RNA
cagccagcctgcaggagggag

SEQ ID NO: 382 - RNA, 21 nt, synthetic construct
  source 1..21: mol_type = other RNA
cacccagcctgcaggagggag

SEQ ID NO: 383 - RNA, 21 nt, synthetic construct
  source 1..21: mol_type = other RNA
acagccagcctgcaggaggga

SEQ ID NO: 384 - RNA, 21 nt, synthetic construct
  source 1..21: mol_type = other RNA
aacacccagcctgcaggaggg

SEQ ID NO: 385 - RNA, 21 nt, synthetic construct
  source 1..21: mol_type = other RNA
aacagccagcctgcaggaggg

SEQ ID NO: 386 - RNA, 21 nt, synthetic construct
  source 1..21: mol_type = other RNA
caacagccagcctgcaggagg

SEQ ID NO: 387 - RNA, 21 nt, synthetic construct
  source 1..21: mol_type = other RNA
caacacccagcctgcaggagg

SEQ ID NO: 388 - RNA, 21 nt, synthetic construct
  source 1..21: mol_type = other RNA
ccaacagccagcctgcaggag

SEQ ID NO: 389 - RNA, 21 nt, synthetic construct
  source 1..21: mol_type = other RNA
ccaacacccagcctgcaggag

SEQ ID NO: 390 - RNA, 21 nt, synthetic construct
  source 1..21: mol_type = other RNA
gccaacagccagcctgcagga

SEQ ID NO: 391 - RNA, 21 nt, synthetic construct
  source 1..21: mol_type = other RNA
ggccaacagccagcctgcagg

SEQ ID NO: 392 - RNA, 21 nt, synthetic construct
  source 1..21: mol_type = other RNA
ggggccaacacccagcctgca

SEQ ID NO: 393 - RNA, 21 nt, synthetic construct
  source 1..21: mol_type = other RNA
ggggccaacagccagcctgca

SEQ ID NO: 394 - RNA, 21 nt, synthetic construct
  source 1..21: mol_type = other RNA
acaggggccaacacccagcct

SEQ ID NO: 395 - RNA, 21 nt, synthetic construct
  source 1..21: mol_type = other RNA
cacaggggccaacagccagcc

SEQ ID NO: 396 - RNA, 21 nt, synthetic construct
  source 1..21: mol_type = other RNA
cacaggggccaacacccagcc

SEQ ID NO: 397 - RNA, 21 nt, synthetic construct
  source 1..21: mol_type = other RNA
gcacaggggccaacacccagc

SEQ ID NO: 398 - RNA, 21 nt, synthetic construct
  source 1..21: mol_type = other RNA
gcagaggggccaacagccagc

SEQ ID NO: 399 - RNA, 21 nt, synthetic construct
  source 1..21: mol_type = other RNA
gcacaggggccaacagccagc

SEQ ID NO: 400 - RNA, 21 nt, synthetic construct
  source 1..21: mol_type = other RNA
gcagaggggccaacacccagc

SEQ ID NO: 401 - RNA, 21 nt, synthetic construct
  source 1..21: mol_type = other RNA
agcacaggggccaacagccag

SEQ ID NO: 402 - RNA, 21 nt, synthetic construct
  source 1..21: mol_type = other RNA
agcacaggggccaacacccag

SEQ ID NO: 403 - RNA, 21 nt, synthetic construct
  source 1..21: mol_type = other RNA
cagcacaggggccaacagcca

SEQ ID NO: 404 - RNA, 21 nt, synthetic construct
  source 1..21: mol_type = other RNA
cagcacaggggccaacaccca

SEQ ID NO: 405 - RNA, 21 nt, synthetic construct
  source 1..21: mol_type = other RNA
ggacagcagaggggccaacag

SEQ ID NO: 406 - RNA, 21 nt, synthetic construct
  source 1..21: mol_type = other RNA
ggacagcagaggggccaacac

SEQ ID NO: 407 - RNA, 21 nt, synthetic construct
  source 1..21: mol_type = other RNA
ggacagcacaggggccaacag

SEQ ID NO: 408 - RNA, 21 nt, synthetic construct
  source 1..21: mol_type = other RNA
ggacagcacaggggccaacac

SEQ ID NO: 409 - RNA, 21 nt, synthetic construct
  source 1..21: mol_type = other RNA
aggacagcagaggggccaaca

SEQ ID NO: 410 - RNA, 21 nt, synthetic construct
  source 1..21: mol_type = other RNA
aggacagcacaggggccaaca

SEQ ID NO: 411 - RNA, 21 nt, synthetic construct
  source 1..21: mol_type = other RNA
caggacagcacaggggccaac

SEQ ID NO: 412 - RNA, 21 nt, synthetic construct
  source 1..21: mol_type = other RNA
caggacagcagaggggccaac

SEQ ID NO: 413 - RNA, 21 nt, synthetic construct
  source 1..21: mol_type = other RNA
atgccatggcatctgtgcatc

SEQ ID NO: 414 - RNA, 21 nt, synthetic construct
  source 1..21: mol_type = other RNA
caggccatggcatctgtgcat

SEQ ID NO: 415 - RNA, 21 nt, synthetic construct
  source 1..21: mol_type = other RNA
catgccatggcatctgtgcat

SEQ ID NO: 416 - RNA, 21 nt, synthetic construct
  source 1..21: mol_type = other RNA
acaggccatggcatctgtgca

SEQ ID NO: 417 - RNA, 21 nt, synthetic construct
  source 1..21: mol_type = other RNA
acatgccatggcatctgtgca

SEQ ID NO: 418 - RNA, 21 nt, synthetic construct
  source 1..21: mol_type = other RNA
cacaggccatggcatctgtgc

SEQ ID NO: 419 - RNA, 21 nt, synthetic construct
  source 1..21: mol_type = other RNA
cacatgccatggcatctgtgc

SEQ ID NO: 420 - RNA, 21 nt, synthetic construct
  source 1..21: mol_type = other RNA
gcacaggccatggcatctgtg

SEQ ID NO: 421 - RNA, 21 nt, synthetic construct
  source 1..21: mol_type = other RNA
gcacatgccatggcatctgtg

SEQ ID NO: 422 - RNA, 21 nt, synthetic construct
  source 1..21: mol_type = other RNA
agcacatgccatggcatctgt

SEQ ID NO: 423 - RNA, 21 nt, synthetic construct
  source 1..21: mol_type = other RNA
cagcacaggccatggcatctg

SEQ ID NO: 424 - RNA, 21 nt, synthetic construct
  source 1..21: mol_type = other RNA
cagcacatgccatggcatctg

SEQ ID NO: 425 - RNA, 21 nt, synthetic construct
  source 1..21: mol_type = other RNA
cgggtgtctagcacccccagc

SEQ ID NO: 426 - RNA, 21 nt, synthetic construct
  source 1..21: mol_type = other RNA
caggtgtctagcacccccagc

SEQ ID NO: 427 - RNA, 21 nt, synthetic construct
  source 1..21: mol_type = other RNA
ccgggtgtctagcacccccag

SEQ ID NO: 428 - RNA, 21 nt, synthetic construct
  source 1..21: mol_type = other RNA
gccgggtgtctagcaccccca

SEQ ID NO: 429 - RNA, 21 nt, synthetic construct
  source 1..21: mol_type = other RNA
tgccgggtgtctagcaccccc

SEQ ID NO: 430 - RNA, 21 nt, synthetic construct
  source 1..21: mol_type = other RNA
tgccaggtgtctagcaccccc

SEQ ID NO: 431 - RNA, 21 nt, synthetic construct
  source 1..21: mol_type = other RNA
gtgccgggtgtctagcacccc

SEQ ID NO: 432 - RNA, 21 nt, synthetic construct
  source 1..21: mol_type = other RNA
gtgccaggtgtctagcacccc

SEQ ID NO: 433 - RNA, 21 nt, synthetic construct
  source 1..21: mol_type = other RNA
ggtgccaggtgtctagcaccc

SEQ ID NO: 434 - RNA, 21 nt, synthetic construct
  source 1..21: mol_type = other RNA
ggtgccgggtgtctagcaccc

SEQ ID NO: 435 - RNA, 21 nt, synthetic construct
  source 1..21: mol_type = other RNA
tggtgccgggtgtctagcacc

SEQ ID NO: 436 - RNA, 21 nt, synthetic construct
  source 1..21: mol_type = other RNA
tggtgccaggtgtctagcacc

SEQ ID NO: 437 - RNA, 21 nt, synthetic construct
  source 1..21: mol_type = other RNA
atggtgccgggtgtctagcac

SEQ ID NO: 438 - RNA, 21 nt, synthetic construct
  source 1..21: mol_type = other RNA
atggtgccaggtgtctagcac

SEQ ID NO: 439 - RNA, 21 nt, synthetic construct
  source 1..21: mol_type = other RNA
aatggtgccaggtgtctagca

SEQ ID NO: 440 - RNA, 21 nt, synthetic construct
  source 1..21: mol_type = other RNA
aatggtgccgggtgtctagca

SEQ ID NO: 441 - RNA, 21 nt, synthetic construct
  source 1..21: mol_type = other RNA
gaatggtgccaggtgtctagc

SEQ ID NO: 442 - RNA, 21 nt, synthetic construct
  source 1..21: mol_type = other RNA
gaatggtgccgggtgtctagc

SEQ ID NO: 443 - RNA, 21 nt, synthetic construct
  source 1..21: mol_type = other RNA
cagcaagcagagctcccaaat

SEQ ID NO: 444 - RNA, 21 nt, synthetic construct
  source 1..21: mol_type = other RNA
gtcggcaagcagagctcccaa

SEQ ID NO: 445 - RNA, 21 nt, synthetic construct
  source 1..21: mol_type = other RNA
gtcagcaagcagagctcccaa

SEQ ID NO: 446 - RNA, 21 nt, synthetic construct
  source 1..21: mol_type = other RNA
agtcagcaagcagagctccca

SEQ ID NO: 447 - RNA, 21 nt, synthetic construct
  source 1..21: mol_type = other RNA
agtcggcaagcagagctccca

SEQ ID NO: 448 - RNA, 21 nt, synthetic construct
  source 1..21: mol_type = other RNA
cagtcggcaagcagagctccc

SEQ ID NO: 449 - RNA, 21 nt, synthetic construct
  source 1..21: mol_type = other RNA
cagtcagcaagcagagctccc

SEQ ID NO: 450 - RNA, 21 nt, synthetic construct
  source 1..21: mol_type = other RNA
ccagtcagcaagcagagctcc

SEQ ID NO: 451 - RNA, 21 nt, synthetic construct
  source 1..21: mol_type = other RNA
ccagtcggcaagcagagctcc

SEQ ID NO: 452 - RNA, 21 nt, synthetic construct
  source 1..21: mol_type = other RNA
gccagtcagcaagcagagctc

SEQ ID NO: 453 - RNA, 21 nt, synthetic construct
  source 1..21: mol_type = other RNA
gccagtcggcaagcagagctc

SEQ ID NO: 454 - RNA, 21 nt, synthetic construct
  source 1..21: mol_type = other RNA
agccagtcagcaagcagagct

SEQ ID NO: 455 - RNA, 21 nt, synthetic construct
  source 1..21: mol_type = other RNA
agccagtcggcaagcagagct

SEQ ID NO: 456 - RNA, 21 nt, synthetic construct
  source 1..21: mol_type = other RNA
cagccagtcagcaagcagagc

SEQ ID NO: 457 - RNA, 21 nt, synthetic construct
  source 1..21: mol_type = other RNA
cagccagtcggcaagcagagc

SEQ ID NO: 458 - RNA, 21 nt, synthetic construct
  source 1..21: mol_type = other RNA
acagccagtcagcaagcagag

SEQ ID NO: 459 - RNA, 21 nt, synthetic construct
  source 1..21: mol_type = other RNA
acagccagtcggcaagcagag

SEQ ID NO: 460 - RNA, 21 nt, synthetic construct
  source 1..21: mol_type = other RNA
atagtcgccaaccttgcctgg

SEQ ID NO: 461 - RNA, 21 nt, synthetic construct
  source 1..21: mol_type = other RNA
acagtcgccaaccttgcctgg

SEQ ID NO: 462 - RNA, 21 nt, synthetic construct
  source 1..21: mol_type = other RNA
gacagtcgccaaccttgcctg

SEQ ID NO: 463 - RNA, 21 nt, synthetic construct
  source 1..21: mol_type = other RNA
gatagtcgccaaccttgcctg

SEQ ID NO: 464 - RNA, 21 nt, synthetic construct
  source 1..21: mol_type = other RNA
tgacagtcgccaaccttgcct

SEQ ID NO: 465 - RNA, 21 nt, synthetic construct
  source 1..21: mol_type = other RNA
tgatagtcgccaaccttgcct

SEQ ID NO: 466 - RNA, 21 nt, synthetic construct
  source 1..21: mol_type = other RNA
atgacagtcgccaaccttgcc

SEQ ID NO: 467 - RNA, 21 nt, synthetic construct
  source 1..21: mol_type = other RNA
atgatagtcgccaaccttgcc

SEQ ID NO: 468 - RNA, 21 nt, synthetic construct
  source 1..21: mol_type = other RNA
catgatagtcgccaaccttgc

SEQ ID NO: 469 - RNA, 21 nt, synthetic construct
  source 1..21: mol_type = other RNA
acatgatagtcgccaaccttg

SEQ ID NO: 470 - RNA, 21 nt, synthetic construct
  source 1..21: mol_type = other RNA
cacatgacagtcgccaacctt

SEQ ID NO: 471 - RNA, 21 nt, synthetic construct
  source 1..21: mol_type = other RNA
cacatgatagtcgccaacctt

SEQ ID NO: 472 - RNA, 21 nt, synthetic construct
  source 1..21: mol_type = other RNA
ccacatgacagtcgccaacct

SEQ ID NO: 473 - RNA, 21 nt, synthetic construct
  source 1..21: mol_type = other RNA
ccacatgatagtcgccaacct

SEQ ID NO: 474 - RNA, 21 nt, synthetic construct
  source 1..21: mol_type = other RNA
gccacatgacagtcgccaacc

SEQ ID NO: 475 - RNA, 21 nt, synthetic construct
  source 1..21: mol_type = other RNA
gccacatgatagtcgccaacc

SEQ ID NO: 476 - RNA, 21 nt, synthetic construct
  source 1..21: mol_type = other RNA
agccacatgacagtcgccaac

SEQ ID NO: 477 - RNA, 21 nt, synthetic construct
  source 1..21: mol_type = other RNA
agccacatgatagtcgccaac

SEQ ID NO: 478 - RNA, 21 nt, synthetic construct
  source 1..21: mol_type = other RNA
cacagagggaggaagcggggg

SEQ ID NO: 479 - RNA, 21 nt, synthetic construct
  source 1..21: mol_type = other RNA
cccacagagggaggaagcggg

SEQ ID NO: 480 - RNA, 21 nt, synthetic construct
  source 1..21: mol_type = other RNA
cccgcagagggaggaagcggg

SEQ ID NO: 481 - RNA, 21 nt, synthetic construct
  source 1..21: mol_type = other RNA
ccccgcagagggaggaagcgg

SEQ ID NO: 482 - RNA, 21 nt, synthetic construct
  source 1..21: mol_type = other RNA
tccccacagagggaggaagcg

SEQ ID NO: 483 - RNA, 21 nt, synthetic construct
  source 1..21: mol_type = other RNA
ctccccgcagagggaggaagc

SEQ ID NO: 484 - RNA, 21 nt, synthetic construct
  source 1..21: mol_type = other RNA
ctccccacagagggaggaagc

SEQ ID NO: 485 - RNA, 21 nt, synthetic construct
  source 1..21: mol_type = other RNA
cctccccgcagagggaggaag

SEQ ID NO: 486 - RNA, 21 nt, synthetic construct
  source 1..21: mol_type = other RNA
cctccccacagagggaggaag

SEQ ID NO: 487 - RNA, 21 nt, synthetic construct
  source 1..21: mol_type = other RNA
tcctccccgcagagggaggaa

SEQ ID NO: 488 - RNA, 21 nt, synthetic construct
  source 1..21: mol_type = other RNA
tcctccccacagagggaggaa

SEQ ID NO: 489 - RNA, 21 nt, synthetic construct
  source 1..21: mol_type = other RNA
gtcctccccgcagagggagga

SEQ ID NO: 490 - RNA, 21 nt, synthetic construct
  source 1..21: mol_type = other RNA
ggtcctccccgcagagggagg

(19) United States
(12) Patent Application Publication
(10) Pub. No.: US 2026/0351343 A1
(43) Pub. Date: Nov. 5, 2026

(54) Allele Preferential RNAi Of Mutant Huntingtin (mtHTT) Using Allele-Specific Inhibitory Nucleic Acid Molecules

(71) Applicant: Regeneron Pharmaceuticals, Inc., Tarrytown, NY (US)

(72) Inventors: Eli A. Stahl, Tarrytown, NY (US); Sahar Gelfman, Tarrytown, NY (US); Neelroop Parikshak, Tarrytown, NY (US); Zebulun Arendsee, Tarrytown, NY (US); Giovanni Coppola, Tarrytown, NY (US); Vijay Kumar Manickavasagam Pounraja, Tarrytown, NY (US); Anthony Marcketta, Tarrytown, NY (US); Goncalo Abecasis, Tarrytown, NY (US); Aris Baras, Tarrytown, NY (US)

(21) Appl. No.: 19/573,145

(22) Filed: Mar. 20, 2026

Related U.S. Application Data
(60) Provisional application No. 63/775,400, filed on Mar. 21, 2025.

(51) Int. Cl. G16B 20/20; G16B 30/00; G16H 20/17

(52) U.S. Cl. CPC G16B 20/20; G16B 30/00; G16H 20/17

(57) ABSTRACT

The present disclosure generally relates to the treatment of subjects having Huntington's disease and having mtHTT or at risk of developing Huntington's disease and having mtHTT, by administering allele-specific inhibitory nucleic acid molecules to the subject, and to methods of identifying subjects having an increased risk of developing Huntington's disease.

### REFERENCE TO SEQUENCE LISTING

This application includes a Sequence Listing submitted electronically as an XML file named 381204599SEQ, created on Mar. 13, 2026, with a size of 432,946 bytes. The Sequence Listing is incorporated herein by reference.

### FIELD

The present disclosure generally relates to the treatment of subjects having Huntington's disease and having mtHTT or at risk of developing Huntington's disease and having mtHTT, by administering allele-specific inhibitory nucleic acid molecules to the subject, and to methods of identifying subjects having an increased risk of developing Huntington's disease.

### BACKGROUND

Huntington's disease (HD) is an autosomal dominant progressive neurodegenerative disorder characterized by loss of striatal neurons. HD affects areas of the brain that help to control voluntary (intentional) movement, as well as other areas. People living with HD develop involuntary jerking or twitching movements known as chorea and abnormal body postures, as well as problems with behavior, emotion, thinking, and/or personality. Adult-onset HD, the most common form of this disorder, usually appears in a person's thirties or forties. Early signs of HD can vary, but often include mild clumsiness or problems with balance or movement, cognitive or psychiatric symptoms (problems with thinking or emotion), and changes in behavior. As the disease progresses, involuntary movements become more pronounced and affected individuals may have trouble walking, speaking, and swallowing. In addition, affected individuals may also experience changes in personality and a decline in thinking and reasoning abilities. Therapeutics can help manage some of the symptoms but cannot slow down or stop the disease as there is no cure for HD. Huntington (HTT) protein is encoded by a 202 kb gene located at the 4p16.3 chromosomal locus. HTT is 3142 amino acids long and is a 347 KDa protein thought to be involved in axonal transport. Mutant Huntingtin (mtHTT) is characterized by an unstable trinucleotide repeat expansion in the HTT gene. Specifically, a polyglutamine encoding CAG repeat in the HTT gene (4:3074877:CAG:(CAG)~) with excessive (more than 36) CAG repeats results in an unstable mutant Huntingtin protein and is the cause of Huntington's Disease (HD).

### SUMMARY

The present disclosure provides methods of treating a subject having Huntington's disease or at risk of developing Huntington's disease, wherein the subject has a pathogenic mutant huntingtin (mtHTT) allele, the methods comprising: a) performing or having performed a sequence analysis on a biological sample obtained from the subject; b) determining from the sequence analysis or having determined from the sequence analysis whether the subject is heterozygous for a first mtHTT variant, wherein a first mtHTT variant targeted allele is in phase with the pathogenic mtHTT allele, wherein the first mtHTT variant targeted allele is the alternate allele of the first mtHTT variant and is in phase with the pathogenic mtHTT allele more often than any other allele in a population of subjects; and i) administering or continuing to administer a first mtHTT variant alternate allele-specific inhibitory nucleic acid molecule to the subject that is heterozygous for the first mtHTT variant targeted allele in phase with the pathogenic mtHTT allele; or ii) performing step c) for the subject that is homozygous for the first mtHTT variant or heterozygous for the first mtHTT variant but the targeted allele is not in phase with the pathogenic mtHTT allele; c) determining from the sequence analysis or having determined from the sequence analysis whether the subject is heterozygous for a subsequent mtHTT variant in which the alternate allele or reference allele is in phase with the pathogenic mtHTT allele; and i) administering or continuing to administer a subsequent mtHTT variant reference allele-specific inhibitory nucleic acid molecule or a subsequent mtHTT variant alternate allele-specific inhibitory nucleic acid molecule, whichever is in phase with the pathogenic mtHTT allele, to the subject that is heterozygous for the subsequent mtHTT variant; or ii) repeating step c) for the subject that is homozygous for the subsequent mtHTT variant until a further subsequent mtHTT variant that is heterozygous is identified for the subject, and administering to the subject or continuing to administer to the subject a further subsequent mtHTT variant reference allele-specific inhibitory nucleic acid molecule or a further subsequent mtHTT variant alternate allele-specific inhibitory nucleic acid molecule, whichever is in phase with the pathogenic mtHTT allele.

The present disclosure also provides methods of treating a subject having Huntington's disease or at risk of developing Huntington's disease, wherein the subject has a pathogenic mutant huntingtin (mtHTT) allele, the methods comprising: a) performing or having performed a sequence analysis on a biological sample obtained from the subject; b) determining from the sequence analysis or having determined from the sequence analysis whether the subject is heterozygous for a first mtHTT variant, wherein the first mtHTT variant targeted allele is in phase with the pathogenic mtHTT allele, wherein the first mtHTT variant targeted allele is the alternate allele of the first mtHTT variant and is in phase with the pathogenic mtHTT allele more often than any other allele in a population of subjects; and i) administering or continuing to administer a first mtHTT variant alternate allele-specific inhibitory nucleic acid molecule to the subject that is heterozygous for the first mtHTT variant targeted allele in phase with the pathogenic mtHTT allele; or ii) performing step c) for the subject that is homozygous for the first mtHTT variant or heterozygous for the first mtHTT variant but the targeted allele is not in phase with the pathogenic mtHTT allele; c) determining from the sequence analysis or having determined from the sequence analysis whether the subject is heterozygous for a first subsequent mtHTT variant and heterozygous for a second subsequent mtHTT variant; and i) administering or continuing to administer a first subsequent mtHTT variant alternate allele-specific/second subsequent mtHTT variant alternate allele-specific inhibitory nucleic acid molecule to the subject that has both the first subsequent mtHTT variant alternate allele and the second subsequent mtHTT variant alternate allele in phase with the pathogenic mtHTT allele; or ii) administering or continuing to administer a first subsequent mtHTT variant reference allele-specific/second subsequent mtHTT variant reference allele-specific inhibitory nucleic acid molecule to the subject that has both the first subsequent mtHTT variant reference allele and the second subsequent mtHTT variant reference allele in phase with the pathogenic mtHTT allele; or iii) administering or continuing to administer a first subsequent mtHTT variant alternate allele-specific/second subsequent mtHTT variant reference allele-specific inhibitory nucleic acid molecule to the subject that has both the first subsequent mtHTT variant alternate allele and the second subsequent mtHTT variant reference allele in phase with the pathogenic mtHTT allele; or iv) administering or continuing to administer a first subsequent mtHTT variant reference allele-specific/second subsequent mtHTT variant alternate allele-specific inhibitory nucleic acid molecule to the subject that has both the first subsequent mtHTT variant reference allele and the second subsequent mtHTT variant alternate allele in phase with the pathogenic mtHTT allele; or ii) repeating step c) for the subject that is homozygous for either the first subsequent mtHTT variant or the second subsequent mtHTT variant until a first further subsequent mtHTT variant or a second further subsequent mtHTT variant that are heterozygous is identified for the subject, and administering to the subject or continuing to administer to the subject: a first further subsequent mtHTT variant alternate allele-specific/second further subsequent mtHTT variant alternate allele-specific inhibitory nucleic acid molecule to the subject that has both the first further subsequent mtHTT variant alternate allele and the second further subsequent mtHTT variant alternate allele in phase with the pathogenic mtHTT allele; or a first further subsequent mtHTT variant reference allele-specific/second further subsequent mtHTT variant reference allele-specific inhibitory nucleic acid molecule to the subject that has both the first further subsequent mtHTT variant reference allele and the second further subsequent mtHTT variant reference allele in phase with the pathogenic mtHTT allele; or a first further subsequent mtHTT variant alternate allele-specific/second further subsequent mtHTT variant reference allele-specific inhibitory nucleic acid molecule to the subject that has both the first further subsequent mtHTT variant alternate allele and the second further subsequent mtHTT variant reference allele in phase with the pathogenic mtHTT allele; or a first further subsequent mtHTT variant reference allele-specific/second further subsequent mtHTT variant alternate allele-specific inhibitory nucleic acid molecule to the subject that has both the first further subsequent mtHTT variant reference allele and the second further subsequent mtHTT variant alternate allele in phase with the pathogenic mtHTT allele. The present disclosure also provides methods of treating a subject having Huntington's disease or at risk of developing Huntington's disease, wherein the subject has a pathogenic mutant huntingtin (mtHTT) allele, the methos comprising: i) administering a first mtHTT variant alternate allele-specific inhibitory nucleic acid molecule to the subject that is heterozygous for a first mtHTT variant targeted allele in phase with the pathogenic mtHTT allele, wherein the first mtHTT variant targeted allele is the alternate allele of the first mtHTT variant and is in phase with the pathogenic mtHTT allele more often than any other allele in a population of subjects; and ii) for the subject that is homozygous for the first mtHTT variant or heterozygous for the first mtHTT variant but the targeted allele is not in phase with the pathogenic mtHTT allele, administering a subsequent mtHTT variant reference allele-specific inhibitory nucleic acid molecule or a subsequent mtHTT variant alternate allele-specific inhibitory nucleic acid molecule, whichever is in phase with the pathogenic mtHTT allele.

The present disclosure also provides methods of treating a subject having Huntington's disease or at risk of developing Huntington's disease, wherein the subject has a pathogenic mutant huntingtin (mtHTT) allele, the methods comprising: i) administering a first mtHTT variant alternate allele-specific inhibitory nucleic acid molecule to the subject that is heterozygous for a first mtHTT variant targeted allele in phase with the pathogenic mtHTT allele, wherein the first mtHTT variant targeted allele is the alternate allele of the first mtHTT variant and is in phase with the pathogenic mtHTT allele more often than any other allele in a population of subjects; and ii) for the subject that is homozygous for the first mtHTT variant or heterozygous for the first mtHTT variant but the targeted allele is not in phase with the pathogenic mtHTT allele: a) administering or continuing to administer a first subsequent mtHTT variant alternate allele-specific/second subsequent mtHTT variant alternate allele-specific inhibitory nucleic acid molecule to the subject that has both the first subsequent mtHTT variant alternate allele and the second subsequent mtHTT variant alternate allele in phase with the pathogenic mtHTT allele; or b) administering or continuing to administer a first subsequent mtHTT variant reference allele-specific/second subsequent mtHTT variant reference allele-specific inhibitory nucleic acid molecule to the subject that has both the first subsequent mtHTT variant reference allele and the second subsequent mtHTT variant reference allele in phase with the pathogenic mtHTT allele; or c) administering or continuing to administer a first subsequent mtHTT variant alternate allele-specific/second subsequent mtHTT variant reference allele-specific inhibitory nucleic acid molecule to the subject that has both the first subsequent mtHTT variant alternate allele and the second subsequent mtHTT variant reference allele in phase with the pathogenic mtHTT allele; or d) administering or continuing to administer a first subsequent mtHTT variant reference allele-specific/second subsequent mtHTT variant alternate allele-specific inhibitory nucleic acid molecule to the subject that has both the first subsequent mtHTT variant reference allele and the second subsequent mtHTT variant alternate allele in phase with the pathogenic mtHTT allele.

The present disclosure also provides methods of identifying a subject having Huntington's disease or at risk of developing Huntington's disease that is a candidate for treatment with an inhibitory nucleic acid molecule, wherein the subject has a pathogenic mutant huntingtin (mtHTT) allele, the methods comprising: a) performing or having performed a sequence analysis on a biological sample obtained from the subject; and i) determining from the sequence analysis or having determined from the sequence analysis whether the subject is heterozygous for a first mtHTT variant, wherein a first mtHTT variant targeted allele is in phase with the pathogenic mtHTT allele, wherein the first mtHTT variant targeted allele is the alternate allele of the first mtHTT variant and is in phase with the pathogenic mtHTT allele more often than any other allele in a population of subjects, wherein the subject is a candidate for treatment with a first mtHTT variant alternate allele-specific inhibitory nucleic acid molecule when the subject is heterozygous for the first mtHTT variant targeted allele in phase with the pathogenic mtHTT allele; or ii) for the subject that is homozygous for the first mtHTT variant, or heterozygous for the first mtHTT variant but the targeted allele is not in phase with the pathogenic mtHTT allele, determining from the sequence analysis or having determined from the sequence analysis whether the subject is heterozygous for a subsequent mtHTT variant in which the alternate allele or reference allele is in phase with the pathogenic mtHTT allele, wherein the subject is a candidate for treatment with a subsequent mtHTT variant reference allele-specific inhibitory nucleic acid molecule or a subsequent mtHTT variant alternate allele-specific inhibitory nucleic acid molecule, whichever is in phase with the pathogenic mtHTT allele, when the subject is heterozygous for the subsequent mtHTT variant.

The present disclosure also provides methods of identifying a subject having Huntington's disease or at risk of developing Huntington's disease that is a candidate for treatment with an inhibitory nucleic acid molecule, wherein the subject has a pathogenic mutant huntingtin (mtHTT) allele, the methods comprising: performing or having performed a sequence analysis on a biological sample obtained from the subject; and i) determining from the sequence analysis or having determined from the sequence analysis whether the subject is heterozygous for a first mtHTT variant, wherein a first mtHTT variant targeted allele is in phase with the pathogenic mtHTT allele, wherein the first mtHTT variant targeted allele is the alternate allele of the first mtHTT variant and is in phase with the pathogenic mtHTT allele more often than any other allele in a population of subjects, wherein the subject is a candidate for treatment with a first mtHTT variant alternate allele-specific inhibitory nucleic acid molecule when the subject is heterozygous for the first mtHTT variant targeted allele in phase with the pathogenic mtHTT allele; or ii) for the subject that is homozygous for the first mtHTT variant, or heterozygous for the first mtHTT variant but the targeted allele is not in phase with the pathogenic mtHTT allele, determining from the sequence analysis or having determined from the sequence analysis whether the subject is heterozygous for a first subsequent mtHTT variant and heterozygous for a second subsequent mtHTT variant, wherein the subject is a candidate for treatment with: a) a first subsequent mtHTT variant alternate allele-specific/second subsequent mtHTT variant alternate allele-specific inhibitory nucleic acid molecule to the subject that has both the first subsequent mtHTT variant alternate allele and the second subsequent mtHTT variant alternate allele in phase with the pathogenic mtHTT allele; b) a first subsequent mtHTT variant reference allele-specific/second subsequent mtHTT variant reference allele-specific inhibitory nucleic acid molecule to the subject that has both the first subsequent mtHTT variant reference allele and the second subsequent mtHTT variant reference allele in phase with the pathogenic mtHTT allele; c) a first subsequent mtHTT variant alternate allele-specific/second subsequent mtHTT variant reference allele-specific inhibitory nucleic acid molecule to the subject that has both the first subsequent mtHTT variant alternate allele and the second subsequent mtHTT variant reference allele in phase with the pathogenic mtHTT allele; or d) a first subsequent mtHTT variant reference allele-specific/second subsequent mtHTT variant alternate allele-specific inhibitory nucleic acid molecule to the subject that has both the first subsequent mtHTT variant reference allele and the second subsequent mtHTT variant alternate allele in phase with the pathogenic mtHTT allele.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows association data for 23 HTT coding variants with MAF>1% (population) and >2% proportion of patients treatable.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded, or can be conjugated to another molecule, such as an antibody. One strand of a nucleic acid also refers to its complement.

As used herein, the term “subject” includes any animal, including mammals. Mammals include, but are not limited to, farm animals (such as, for example, horses, cows, and pigs), companion animals (such as, for example, dogs and cats), laboratory animals (such as, for example, mice, rats, and rabbits), and non-human primates. In some embodiments, the subject is a human. In some embodiments, the human is a patient under the care of a physician.

As used herein, the term “mtHTT variant” means any HTT gene that has an alteration in its genetic sequence, thus producing a reference allele (i.e., wild type) and an alternate allele. In some embodiments, the reference allele is in phase with the pathogenic mtHTT allele. In some embodiments, the alternate allele is in phase with the pathogenic mtHTT allele.

The huntingtin (HTT) gene is large, spanning 180 kb and consisting of 67 exons. The HTT gene comprises a trinucleotide (CAG) repeat (4:3074877:CAG:(CAG)ₙ), which translates as a polyglutamine repeat in the protein product. A range of trinucleotide repeats (i.e., n=9-35) has been identified in normal controls. The HTT gene is located at chromosome 4 (i.e., positions 3,074,681-3,243,957) using the nucleotide sequence of the HTT reference genomic nucleic acid molecule in the GRCh38/hg38 human genome assembly (see, ENSG00000197386.14, ENST00000355072.11 annotated in the Ensembl database (URL: world wide web at “https://www.ncbi.nlm.nih.gov/nuccore/NM_002111.8.”)) as a reference sequence. The sequence provided in this transcript for the HTT genomic nucleic acid molecule is only an exemplary sequence. Other sequences for the HTT genomic nucleic acid molecule are also possible (URL: world wide web at “useast.ensembl.org/Homo_sapiens/Gene/Summary?g=ENSG00000197386;r=4:3041363-3243957; transcript=ENST00000355072.11”).

In the embodiments described herein, the subject has a pathogenic mtHTT allele, which is expanded and unstable. The CAG repeat number is 37 or more, up to 120 repeats or more (i.e., 4:3074877:CAG:(CAG)_{n>36}). One skilled in the art can determine whether a particular subject has a pathogenic mtHTT allele by obtaining a biological sample from the subject and examining the HTT nucleotide sequence by, for example, sequencing.

In any of the embodiments described herein, the subject can have Huntington's disease. In any of the embodiments described herein, the subject can be at risk of developing Huntington's disease.

The present disclosure provides methods of treating a subject having Huntington's disease or at risk of developing Huntington's disease. The subject has a pathogenic mutant huntingtin (mtHTT) allele. The methods comprise performing or having performed a sequence analysis on a biological sample obtained from the subject. The methods also comprise determining from the sequence analysis or having determined from the sequence analysis whether the subject is heterozygous for a first mtHTT variant, wherein a first mtHTT variant targeted allele is in phase with the pathogenic mtHTT allele. The first mtHTT variant targeted allele is the alternate allele of the first mtHTT variant and is in phase with the pathogenic mtHTT allele more often than any other allele in a population of subjects. For a subject that is heterozygous for the first mtHTT variant targeted allele in phase with the pathogenic mtHTT allele, the methods comprise administering or continuing to administer a first mtHTT variant alternate allele-specific inhibitory nucleic acid molecule to the subject. For a subject that is homozygous for the first mtHTT variant or heterozygous for the first mtHTT variant but the targeted allele is not in phase with the pathogenic mtHTT allele, the methods comprise determining from the sequence analysis or having determined from the sequence analysis whether the subject is heterozygous for a subsequent mtHTT variant in which the alternate allele or reference allele is in phase with the pathogenic mtHTT allele. For a subject that is heterozygous for the subsequent mtHTT variant, the methods comprise administering or continuing to administer a subsequent mtHTT variant reference allele-specific inhibitory nucleic acid molecule or a subsequent mtHTT variant alternate allele-specific inhibitory nucleic acid molecule, whichever is in phase with the pathogenic mtHTT allele. For a subject that is homozygous for the subsequent mtHTT variant, the methods comprise determining from the sequence analysis or having determined from the sequence analysis whether the subject is heterozygous for a further subsequent mtHTT variant in which the alternate allele or reference allele is in phase with the pathogenic mtHTT allele until a further subsequent mtHTT variant that is heterozygous is identified for the subject, at which time the methods comprise administering to the subject or continuing to administer to the subject a further subsequent mtHTT variant reference allele-specific inhibitory nucleic acid molecule or a further subsequent mtHTT variant alternate allele-specific inhibitory nucleic acid molecule, whichever is in phase with the pathogenic mtHTT allele.

The present disclosure also provides methods of treating a subject having Huntington's disease or at risk of developing Huntington's disease. The subject has a pathogenic mutant huntingtin (mtHTT) allele. For a subject that is heterozygous for a first mtHTT variant targeted allele in phase with the pathogenic mtHTT allele, wherein the first mtHTT variant targeted allele is the alternate allele of the first mtHTT variant and is in phase with the pathogenic mtHTT allele more often than any other allele in a population of subjects, the methods comprise administering a first mtHTT variant alternate allele-specific inhibitory nucleic acid molecule to the subject. For a subject that is homozygous for the first mtHTT variant or heterozygous for the first mtHTT variant but the targeted allele is not in phase with the pathogenic mtHTT allele, the methods comprise administering a subsequent mtHTT variant reference allele-specific inhibitory nucleic acid molecule or a subsequent mtHTT variant alternate allele-specific inhibitory nucleic acid molecule, whichever is in phase with the pathogenic mtHTT allele.

Table 1 shows variants in the HTT mRNA and the proportion of patients with the indicated target allele on the same chromosome as the pathogenic triplet repeat expansion.

TABLE 1

| | | | Prop. | Propor- |
|---|---|---|---|---|
| | | Propor- | Het | tion |
| | | tion | Cases | of |
| | Target_{—} | Hets in | In | Patients |
| Variant name | allele | Cases | Phase | Targeted |
| 4:3074723:C:T | ALT | 0.035398 | 0 | 0 |
| 4:3074723:C:T | REF | 0.035398 | 1 | 0.035398 |
| 4:3115441:C:G | ALT | 0.044248 | 0 | 0 |
| 4:3115441:C:G | REF | 0.044248 | 1 | 0.044248 |
| 4:3121347:C:T | ALT | 0.088496 | 0.7 | 0.061947 |
| 4:3121347:C:T | REF | 0.088496 | 0.3 | 0.026549 |
| 4:3135947:G:A | ALT | 0.141593 | 0.625 | 0.088496 |
| 4:3135947:G:A | REF | 0.141593 | 0.375 | 0.053097 |
| 4:3146926:T:G | ALT | 0.017699 | 0.5 | 0.00885 |
| 4:3146926:T:G | REF | 0.017699 | 0.5 | 0.00885 |
| 4:3160329:C:T | ALT | 0.389381 | 0.181818 | 0.070796 |
| 4:3160329:C:T | REF | 0.389381 | 0.818182 | 0.318584 |
| 4:3178294:T:C | ALT | 0.486726 | 0.290909 | 0.141593 |
| 4:3178294:T:C | REF | 0.486726 | 0.709091 | 0.345133 |
| 4:3187820:C:A | ALT | 0.150442 | 0.352941 | 0.053097 |
| 4:3187820:C:A | REF | 0.150442 | 0.647059 | 0.097345 |
| 4:3212105:G:A | ALT | 0.39823 | 0.2 | 0.079646 |
| 4:3212105:G:A | REF | 0.39823 | 0.8 | 0.318584 |
| 4:3212148:T:C | ALT | 0.141593 | 0.375 | 0.053097 |
| 4:3212148:T:C | REF | 0.141593 | 0.625 | 0.088496 |
| 4:3212714:G:A | ALT | 0.017699 | 0 | 0 |
| 4:3212714:G:A | REF | 0.017699 | 1 | 0.017699 |
| 4:3214108:T:C | ALT | 0.460177 | 0.192308 | 0.088496 |
| 4:3214108:T:C | REF | 0.460177 | 0.807692 | 0.371681 |
| 4:3225692:A:G | ALT | 0.389381 | 0.181818 | 0.070796 |
| 4:3225692:A:G | REF | 0.389381 | 0.818182 | 0.318584 |
| 4:3228683:AGAG:A | ALT | 0.442478 | 0.82 | 0.362832 |
| 4:3228683:AGAG:A | REF | 0.442478 | 0.18 | 0.079646 |
| 4:3229934:G:A | ALT | 0.115044 | 0.307692 | 0.035398 |
| 4:3229934:G:A | REF | 0.115044 | 0.692308 | 0.079646 |
| 4:3233253:G:A | ALT | 0.40708 | 0.173913 | 0.070797 |
| 4:3233253:G:A | REF | 0.40708 | 0.826087 | 0.336283 |
| 4:3240118:C:T | ALT | 0.442478 | 0.82 | 0.362832 |
| 4:3240118:C:T | REF | 0.442478 | 0.18 | 0.079646 |
| 4:3240373:G:A | ALT | 0.40708 | 0.173913 | 0.070797 |
| 4:3240373:G:A | REF | 0.40708 | 0.826087 | 0.336283 |
| 4:3240421:C:G | ALT | 0.39823 | 0.155556 | 0.061947 |
| 4:3240421:C:G | REF | 0.39823 | 0.844444 | 0.336283 |
| 4:3240433:C:G | ALT | 0.141593 | 0.4375 | 0.061947 |
| 4:3240433:C:G | REF | 0.141593 | 0.5625 | 0.079646 |
| 4:3240545:C:A | ALT | 0.141593 | 0.4375 | 0.061947 |
| 4:3240545:C:A | REF | 0.141593 | 0.5625 | 0.079646 |
| 4:3240580:C:T | ALT | 0.150442 | 0.470588 | 0.070796 |
| 4:3240580:C:T | REF | 0.150442 | 0.529412 | 0.079646 |
| 4:3241211:G:T | ALT | 0.017699 | 0.5 | 0.00885 |
| 4:3241211:G:T | REF | 0.017699 | 0.5 | 0.00885 |
| 4:3241491:C:T | ALT | 0.40708 | 0.173913 | 0.070797 |
| 4:3241491:C:T | REF | 0.40708 | 0.826087 | 0.336283 |
| 4:3241563:G:A | ALT | 0.106195 | 0.833333 | 0.088496 |
| 4:3241563:G:A | REF | 0.106195 | 0.166667 | 0.017699 |
| 4:3242045:TG:T | ALT | 0.150442 | 0.411765 | 0.061947 |
| 4:3242045:TG:T | REF | 0.150442 | 0.588235 | 0.088496 |
| 4:3243330:C:T | ALT | 0.318584 | 0.555556 | 0.176991 |
| 4:3243330:C:T | REF | 0.318584 | 0.444444 | 0.141593 |

In any of the methods of treatment described herein, the first mtHTT variant alternate allele-specific inhibitory nucleic acid molecule, subsequent mtHTT variant reference allele-specific inhibitory nucleic acid molecule, subsequent mtHTT variant alternate allele-specific inhibitory nucleic acid molecule, further subsequent mtHTT variant reference allele-specific inhibitory nucleic acid molecule, and further subsequent mtHTT variant alternate allele-specific inhibitory nucleic acid molecule comprise an antisense nucleic acid molecule, a small interfering RNA (siRNA), or a short hairpin RNA (shRNA).

In any of the methods of treatment described herein, the first mtHTT variant alternate allele-specific inhibitory nucleic acid molecule, subsequent mtHTT variant reference allele-specific inhibitory nucleic acid molecule, subsequent mtHTT variant alternate allele-specific inhibitory nucleic acid molecule, further subsequent mtHTT variant reference allele-specific inhibitory nucleic acid molecule, and further subsequent mtHTT variant alternate allele-specific inhibitory nucleic acid molecule comprise an siRNA.

In any of the methods of treatment described herein, the first mtHTT variant alternate allele-specific inhibitory nucleic acid molecule, subsequent mtHTT variant reference allele-specific inhibitory nucleic acid molecule, subsequent mtHTT variant alternate allele-specific inhibitory nucleic acid molecule, further subsequent mtHTT variant reference allele-specific inhibitory nucleic acid molecule, and further subsequent mtHTT variant alternate allele-specific inhibitory nucleic acid molecule comprise an antisense nucleic acid molecule.

In any of the methods of treatment described herein, the first mtHTT variant comprises 4:3228683:AGAG:A; and i) the first mtHTT variant alternate-specific inhibitory nucleic acid molecule comprises an antisense nucleic acid molecule comprising the nucleotide sequence of SEQ ID NO:334, SEQ ID NO:335, or SEQ ID NO:336; or ii) the first mtHTT variant alternate-specific inhibitory nucleic acid molecule comprises an siRNA comprising the nucleotide sequences of SEQ ID NOs:89/334, SEQ ID NOs:90/335, or SEQ ID NOs:91/336.

In any of the methods of treatment described herein, the subsequent mtHTT variant or further subsequent mtHTT variant comprises 4:3240421:C:G; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:380, SEQ ID NO:382, SEQ ID NO:384, SEQ ID NO:387, SEQ ID NO:389, or SEQ ID NO:392; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:135/380, SEQ ID NOs:137/382, SEQ ID NOs:139/384, SEQ ID NOs:142/387, SEQ ID NOs:144/389, or SEQ ID NOs:147/392; iii) the subsequent mtHTT variant reference-specific inhibitory nucleic acid molecule or further subsequent mtHTT variant reference-specific inhibitory nucleic acid molecule comprises an antisense nucleic acid molecule comprising the nucleotide sequence of SEQ ID NO:379, SEQ ID NO:381, SEQ ID NO:383, SEQ ID NO:385, SEQ ID NO:386, SEQ ID NO:388, SEQ ID NO:390, SEQ ID NO:391, or SEQ ID NO:393; or iv) the subsequent mtHTT variant reference-specific inhibitory nucleic acid molecule or further subsequent mtHTT variant reference-specific inhibitory nucleic acid molecule comprises an siRNA comprising the nucleotide sequences of SEQ ID NOs:134/379, SEQ ID NOs:136/381, SEQ ID NOs:138/383, SEQ ID NOs:140/385, SEQ ID NOs:141/386, SEQ ID NOs:143/388, SEQ ID NOs:145/390, SEQ ID NOs:146/391, or SEQ ID NOs:148/393.

In any of the methods of treatment described herein, the subsequent mtHTT variant or further subsequent mtHTT variant comprises 4:3074723:C:T; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:247, SEQ ID NO:249, SEQ ID NO:251, SEQ ID NO:253, SEQ ID NO:255, SEQ ID NO:257, SEQ ID NO:259, SEQ ID NO:261, or SEQ ID NO:263; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:2/247, SEQ ID NOs:4/249, SEQ ID NOs:6/251, SEQ ID NOs:8/253, SEQ ID NOs:10/255, SEQ ID NOs:12/257, SEQ ID NOs:14/259, SEQ ID NOs:16/261, or SEQ ID NOs:18/263; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:246, SEQ ID NO:248, SEQ ID NO:250, SEQ ID NO:252, SEQ ID NO:254, SEQ ID NO:256, SEQ ID NO:258, SEQ ID NO:260, or SEQ ID NO:262; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:1/246, SEQ ID NOs:3/248, SEQ ID NOs:5/250, SEQ ID NOs:7/252, SEQ ID NOs:9/254, SEQ ID NOs:11/256, SEQ ID NOs:13/258, SEQ ID NOs:15/260, or SEQ ID NOs:17/262.

In any of the methods of treatment described herein, the subsequent mtHTT variant or further subsequent mtHTT variant comprises 4:3115441:C:G; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:265, SEQ ID NO:266, SEQ ID NO:268, SEQ ID NO:270, SEQ ID NO:272, SEQ ID NO:274, SEQ ID NO:276, or SEQ ID NO:278; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:20/265, SEQ ID NOs:21/266, SEQ ID NOs:23/268, SEQ ID NOs:25/270, SEQ ID NOs:27/272, SEQ ID NOs:29/274, SEQ ID NOs:31/276, or SEQ ID NOs:33/278; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:264, SEQ ID NO:267, SEQ ID NO:269, SEQ ID NO:271, SEQ ID NO:273, SEQ ID NO:275, or SEQ ID NO:277; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:19/264, SEQ ID NOs:22/267, SEQ ID NOs:24/269, SEQ ID NOs:26/271, SEQ ID NOs:28/273, SEQ ID NOs:30/275, or SEQ ID NOs:32/277.

In any of the methods of treatment described herein, the subsequent mtHTT variant or further subsequent mtHTT variant comprises 4:3160329:C:T; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:279, SEQ ID NO:280, SEQ ID NO:282, SEQ ID NO:284, SEQ ID NO:286, or SEQ ID NO:287; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:34/279, SEQ ID NOs:35/280, SEQ ID NOs:37/282, SEQ ID NOs:39/284, SEQ ID NOs:41/286, or SEQ ID NOs:42/287; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:281, SEQ ID NO:283, SEQ ID NO:285, or SEQ ID NO:288; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:36/281, SEQ ID NOs:38/283, SEQ ID NOs:40/285, or SEQ ID NOs:43/288.

In any of the methods of treatment described herein, the subsequent mtHTT variant or further subsequent mtHTT variant comprises 4:3187820:C:A; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:289, SEQ ID NO:291, SEQ ID NO:294, SEQ ID NO:296, or SEQ ID NO:297; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:44/289, SEQ ID NOs:46/291, SEQ ID NOs:49/294, SEQ ID NOs:51/296, or SEQ ID NOs:52/297; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:290, SEQ ID NO:292, SEQ ID NO:293, SEQ ID NO:295, or SEQ ID NO:298; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:45/290, SEQ ID NOs:47/292, SEQ ID NOs:48/293, SEQ ID NOs:50/295, or SEQ ID NOs:53/298.

In any of the methods of treatment described herein, the subsequent mtHTT variant or further subsequent mtHTT variant comprises 4:3212105:G:A; and i) the subsequent mtHTT variant reference-specific inhibitory nucleic acid molecule or further subsequent mtHTT variant reference-specific inhibitory nucleic acid molecule comprises an antisense nucleic acid molecule comprising the nucleotide sequence of SEQ ID NO:299, SEQ ID NO:300, SEQ ID NO:301, SEQ ID NO:302, or SEQ ID NO:303; or ii) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:54/299, SEQ ID NOs:55/300, SEQ ID NOs:56/301, SEQ ID NOs:57/302, or SEQ ID NOs:58/303.

In any of the methods of treatment described herein, the subsequent mtHTT variant or further subsequent mtHTT variant comprises 4:3229934:G:A; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:313, SEQ ID NO:315, SEQ ID NO:316, SEQ ID NO:317, SEQ ID NO:320, SEQ ID NO:322, or SEQ ID NO:325; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:68/313, SEQ ID NOs:70/315, SEQ ID NOs:71/316, SEQ ID NOs:72/317, SEQ ID NOs:75/320, SEQ ID NOs:77/322, or SEQ ID NOs:80/325; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:312, SEQ ID NO:314, SEQ ID NO:318, SEQ ID NO:319, SEQ ID NO:321, SEQ ID NO:323, or SEQ ID NO:324; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:67/312, SEQ ID NOs:69/314, SEQ ID NOs:73/318, SEQ ID NOs:74/319, SEQ ID NOs:76/321, SEQ ID NOs:78/323, or SEQ ID NOs:79/324.

In any of the methods of treatment described herein, the subsequent mtHTT variant or further subsequent mtHTT variant comprises 4:3225692:A:G; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:326, SEQ ID NO:328, or SEQ ID NO:330; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:81/326, SEQ ID NOs:83/328, or SEQ ID NOs:85/330; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:327, SEQ ID NO:329, SEQ ID NO:331, or SEQ ID NO:332; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:82/327, SEQ ID NOs:84/329, SEQ ID NOs:86/331, or SEQ ID NOs:87/332.

In any of the methods of treatment described herein, the subsequent mtHTT variant or further subsequent mtHTT variant comprises 4:3233253:G:A; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:338, SEQ ID NO:339, SEQ ID NO:341, SEQ ID NO:345, SEQ ID NO:346, SEQ ID NO:349, or SEQ ID NO:351; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:93/338, SEQ ID NOs:94/339, SEQ ID NOs:96/341, SEQ ID NOs:100/345, SEQ ID NOs:101/346, SEQ ID NOs:104/349, or SEQ ID NOs:106/351; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:337, SEQ ID NO:340, SEQ ID NO:342, SEQ ID NO:343, SEQ ID NO:344, SEQ ID NO:347, SEQ ID NO:348, or SEQ ID NO:350; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:92/337, SEQ ID NOs:95/340, SEQ ID NOs:97/342, SEQ ID NOs:98/343, SEQ ID NOs:99/344, SEQ ID NOs:102/347, SEQ ID NOs:103/348, or SEQ ID NOs:105/350.

In any of the methods of treatment described herein, the subsequent mtHTT variant or further subsequent mtHTT variant comprises 4:3240373:G:A; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:370, SEQ ID NO:372, SEQ ID NO:373, SEQ ID NO:376, SEQ ID NO:377, or SEQ ID NO:378; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:125/370, SEQ ID NOs:127/372, SEQ ID NOs:128/373, SEQ ID NOs:131/376, SEQ ID NOs:132/377, or SEQ ID NO:133/378; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:369, SEQ ID NO:371, SEQ ID NO:374, or SEQ ID NO:375; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:124/369, SEQ ID NOs:126/371, SEQ ID NOs:129/374, or SEQ ID NOs:130/375.

In any of the methods of treatment described herein, the subsequent mtHTT variant or further subsequent mtHTT variant comprises 4:3240433:C:G; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:410 or SEQ ID NO:411; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:165/410 or SEQ ID NOs:166/411; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:409 or SEQ ID NO:412; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:164/409 or SEQ ID NOs:167/412.

In any of the methods of treatment described herein, the subsequent mtHTT variant or further subsequent mtHTT variant comprises 4:3240545:C:A; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:413, SEQ ID NO:415, SEQ ID NO:417, SEQ ID NO:419, SEQ ID NO:421, SEQ ID NO:422, or SEQ ID NO:424; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:168/413, SEQ ID NOs:170/415, SEQ ID NOs:172/417, SEQ ID NOs:174/419, SEQ ID NOs:176/421, SEQ ID NOs:177/422, or SEQ ID NOs:179/424; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:414, SEQ ID NO:416, SEQ ID NO:418, SEQ ID NO:420, or SEQ ID NO:423; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:169/414, SEQ ID NOs:171/416, SEQ ID NOs:173/418, SEQ ID NOs:175/420, or SEQ ID NOs:178/423.

In any of the methods of treatment described herein, the subsequent mtHTT variant or further subsequent mtHTT variant comprises 4:3240580:C:T; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:426, SEQ ID NO:430, SEQ ID NO:432, SEQ ID NO:433, SEQ ID NO:436, SEQ ID NO:438, SEQ ID NO:439, or SEQ ID NO:441; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:181/426, SEQ ID NOs:185/430, SEQ ID NOs:187/432, SEQ ID NOs:188/433, SEQ ID NOs:191/436, SEQ ID NOs:193/438, SEQ ID NOs:194/439, or SEQ ID NOs:196/441; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:425, SEQ ID NO:427, SEQ ID NO:428, SEQ ID NO:429, SEQ ID NO:431, SEQ ID NO:434, SEQ ID NO:435, SEQ ID NO:437, SEQ ID NO:440, or SEQ ID NO:442; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:180/425, SEQ ID NOs:182/427, SEQ ID NOs:183/428, SEQ ID NOs:184/429, SEQ ID NOs:186/431, SEQ ID NOs:189/434, SEQ ID NOs:190/435, SEQ ID NOs:192/437, SEQ ID NOs:195/440, or SEQ ID NOs:197/442.

In any of the methods of treatment described herein, the subsequent mtHTT variant or further subsequent mtHTT variant comprises 4:3241491:C:T; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:443, SEQ ID NO:445, SEQ ID NO:446, SEQ ID NO:449, SEQ ID NO:450, SEQ ID NO:452, SEQ ID NO:454, SEQ ID NO:456, or SEQ ID NO:458; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:198/443, SEQ ID NOs:200/445, SEQ ID NOs:201/446, SEQ ID NOs:204/449, SEQ ID NOs:205/450, SEQ ID NOs:207/452, SEQ ID NOs:209/454, SEQ ID NOs:211/456, or SEQ ID NOs:213/458; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:444, SEQ ID NO:447, SEQ ID NO:448, SEQ ID NO:451, SEQ ID NO:453, SEQ ID NO:455, SEQ ID NO:457, or SEQ ID NO:459; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:199/444, SEQ ID NOs:202/447, SEQ ID NOs:203/448, SEQ ID NOs:206/451, SEQ ID NOs:208/453, SEQ ID NOs:210/455, SEQ ID NOs:212/457, or SEQ ID NOs:214/459.

In any of the methods of treatment described herein, the subsequent mtHTT variant or further subsequent mtHTT variant comprises 4:3241563:G:A; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:460, SEQ ID NO:463, SEQ ID NO:465, SEQ ID NO:467, SEQ ID NO:468, SEQ ID NO:469, SEQ ID NO:471, SEQ ID NO:473, SEQ ID NO:475, or SEQ ID NO:477; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:215/460, SEQ ID NOs:218/463, SEQ ID NOs:220/465, SEQ ID NOs:222/467, SEQ ID NOs:223/468, SEQ ID NOs:224/469, SEQ ID NOs:226/471, SEQ ID NOs:228/473, SEQ ID NOs:230/475, or SEQ ID NOs:232/477; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:461, SEQ ID NO:462, SEQ ID NO:464, SEQ ID NO:466, SEQ ID NO:470, SEQ ID NO:472, SEQ ID NO:474, or SEQ ID NO:476; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:216/461, SEQ ID NOs:217/462, SEQ ID NOs:219/464, SEQ ID NOs:221/466, SEQ ID NOs:225/470, SEQ ID NOs:227/472, SEQ ID NOs:229/474, or SEQ ID NOs:231/476.

In any of the methods of treatment described herein, the subsequent mtHTT variant or further subsequent mtHTT variant comprises 4:3243330:C:T; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:478, SEQ ID NO:479, SEQ ID NO:482, SEQ ID NO:484, SEQ ID NO:486, or SEQ ID NO:488; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:233/478, SEQ ID NOs:234/479, SEQ ID NOs:237/482, SEQ ID NOs:239/484, SEQ ID NOs:241/486, or SEQ ID NOs:243/488; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:480, SEQ ID NO:481, SEQ ID NO:483, SEQ ID NO:485, SEQ ID NO:487, SEQ ID NO:489, or SEQ ID NO:490; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:235/480, SEQ ID NOs:236/481, SEQ ID NOs:238/483, SEQ ID NOs:240/485, SEQ ID NOs:242/487, SEQ ID NOs:244/489, or SEQ ID NOs:245/490.

The present disclosure also provides methods of treating a subject having Huntington's disease or at risk of developing Huntington's disease. The subject has a pathogenic mutant huntingtin (mtHTT) allele. The methods comprise performing or having performed a sequence analysis on a biological sample obtained from the subject. The methods comprise determining from the sequence analysis or having determined from the sequence analysis whether the subject is heterozygous for a first mtHTT variant, wherein the first mtHTT variant targeted allele is in phase with the pathogenic mtHTT allele. The first mtHTT variant targeted allele is the alternate allele of the first mtHTT variant and is in phase with the pathogenic mtHTT allele more often than any other allele in a population of subjects. For a subject that is heterozygous for the first mtHTT variant targeted allele in phase with the pathogenic mtHTT allele, the methods comprise administering or continuing to administer a first mtHTT variant alternate allele-specific inhibitory nucleic acid molecule to the subject. For a subject that is homozygous for the first mtHTT variant or heterozygous for the first mtHTT variant but the targeted allele is not in phase with the pathogenic mtHTT allele, the methods comprise determining from the sequence analysis or having determined from the sequence analysis whether the subject is heterozygous for a first subsequent mtHTT variant and heterozygous for a second subsequent mtHTT variant. For a subject that has both the first subsequent mtHTT variant alternate allele and the second subsequent mtHTT variant alternate allele in phase with the pathogenic mtHTT allele, the methods comprise administering or continuing to administer a first subsequent mtHTT variant alternate allele-specific/second subsequent mtHTT variant alternate allele-specific inhibitory nucleic acid molecule (i.e., a single inhibitory nucleic acid molecule) to the subject. For a subject that has both the first subsequent mtHTT variant reference allele and the second subsequent mtHTT variant reference allele in phase with the pathogenic mtHTT allele, the methods comprise administering or continuing to administer a first subsequent mtHTT variant reference allele-specific/second subsequent mtHTT variant reference allele-specific inhibitory nucleic acid molecule to the subject. For the subject that has both the first subsequent mtHTT variant alternate allele and the second subsequent mtHTT variant reference allele in phase with the pathogenic mtHTT allele, the methods comprise administering or continuing to administer a first subsequent mtHTT variant alternate allele-specific/second subsequent mtHTT variant reference allele-specific inhibitory nucleic acid molecule to the subject. For the subject that has both the first subsequent mtHTT variant reference allele and the second subsequent mtHTT variant alternate allele in phase with the pathogenic mtHTT allele, the methods comprise administering or continuing to administer a first subsequent mtHTT variant reference allele-specific/second subsequent mtHTT variant alternate allele-specific inhibitory nucleic acid molecule to the subject. For a subject that is homozygous for either the first subsequent mtHTT variant or the second subsequent mtHTT, the methods comprise determining from the sequence analysis or having determined from the sequence analysis whether the subject is heterozygous for a first further subsequent mtHTT variant and a second further subsequent mtHTT variant until a first further subsequent mtHTT variant and a second further subsequent mtHTT variant is identified for the subject, at which time the methods comprise administering to the subject or continuing to administer to the subject: i) a first further subsequent mtHTT variant alternate allele-specific/second further subsequent mtHTT variant alternate allele-specific inhibitory nucleic acid molecule to the subject that has both the first further subsequent mtHTT variant alternate allele and the second further subsequent mtHTT variant alternate allele in phase with the pathogenic mtHTT allele; or ii) a first further subsequent mtHTT variant reference allele-specific/second further subsequent mtHTT variant reference allele-specific inhibitory nucleic acid molecule to the subject that has both the first further subsequent mtHTT variant reference allele and the second further subsequent mtHTT variant reference allele in phase with the pathogenic mtHTT allele; or iii) a first further subsequent mtHTT variant alternate allele-specific/second further subsequent mtHTT variant reference allele-specific inhibitory nucleic acid molecule to the subject that has both the first further subsequent mtHTT variant alternate allele and the second further subsequent mtHTT variant reference allele in phase with the pathogenic mtHTT allele; or iv) a first further subsequent mtHTT variant reference allele-specific/second further subsequent mtHTT variant alternate allele-specific inhibitory nucleic acid molecule to the subject that has both the first further subsequent mtHTT variant reference allele and the second further subsequent mtHTT variant alternate allele in phase with the pathogenic mtHTT allele.

The present disclosure also provides methods of treating a subject having Huntington's disease or at risk of developing Huntington's disease. The subject has a pathogenic mutant huntingtin (mtHTT) allele. For a subject that is heterozygous for a first mtHTT variant targeted allele in phase with the pathogenic mtHTT allele, wherein the first mtHTT variant targeted allele is the alternate allele of the first mtHTT variant and is in phase with the pathogenic mtHTT allele more often than any other allele in a population of subjects, the methods comprise administering a first mtHTT variant alternate allele-specific inhibitory nucleic acid molecule to the subject. For a subject that is homozygous for the first mtHTT variant or heterozygous for the first mtHTT variant but the targeted allele is not in phase with the pathogenic mtHTT allele, the methods comprise: a) administering or continuing to administer a first subsequent mtHTT variant alternate allele-specific/second subsequent mtHTT variant alternate allele-specific inhibitory nucleic acid molecule to the subject that has both the first subsequent mtHTT variant alternate allele and the second subsequent mtHTT variant alternate allele in phase with the pathogenic mtHTT allele; or b) administering or continuing to administer a first subsequent mtHTT variant reference allele-specific/second subsequent mtHTT variant reference allele-specific inhibitory nucleic acid molecule to the subject that has both the first subsequent mtHTT variant reference allele and the second subsequent mtHTT variant reference allele in phase with the pathogenic mtHTT allele; or c) administering or continuing to administer a first subsequent mtHTT variant alternate allele-specific/second subsequent mtHTT variant reference allele-specific inhibitory nucleic acid molecule to the subject that has both the first subsequent mtHTT variant alternate allele and the second subsequent mtHTT variant reference allele in phase with the pathogenic mtHTT allele; or d) administering or continuing to administer a first subsequent mtHTT variant reference allele-specific/second subsequent mtHTT variant alternate allele-specific inhibitory nucleic acid molecule to the subject that has both the first subsequent mtHTT variant reference allele and the second subsequent mtHTT variant alternate allele in phase with the pathogenic mtHTT allele.

In any of the methods of treatment described herein, the first mtHTT variant alternate allele-specific inhibitory nucleic acid molecule, first subsequent mtHTT variant alternate allele-specific/second subsequent mtHTT variant alternate allele-specific inhibitory nucleic acid molecule, first subsequent mtHTT variant reference allele-specific/second subsequent mtHTT variant reference allele-specific inhibitory nucleic acid molecule, first subsequent mtHTT variant alternate allele-specific/second subsequent mtHTT variant reference allele-specific inhibitory nucleic acid molecule, first subsequent mtHTT variant reference allele-specific/second subsequent mtHTT variant alternate allele-specific inhibitory nucleic acid molecule, first further subsequent mtHTT variant alternate allele-specific/second further subsequent mtHTT variant alternate allele-specific inhibitory nucleic acid molecule, first further subsequent mtHTT variant reference allele-specific/second further subsequent mtHTT variant reference allele-specific inhibitory nucleic acid molecule, first further subsequent mtHTT variant alternate allele-specific/second further subsequent mtHTT variant reference allele-specific inhibitory nucleic acid molecule, and first further subsequent mtHTT variant reference allele-specific/second further subsequent mtHTT variant alternate allele-specific inhibitory nucleic acid molecule comprise an antisense nucleic acid molecule, a small interfering RNA (siRNA), or a short hairpin RNA (shRNA).

In any of the methods of treatment described herein, the first mtHTT variant alternate allele-specific inhibitory nucleic acid molecule, first subsequent mtHTT variant alternate allele-specific/second subsequent mtHTT variant alternate allele-specific inhibitory nucleic acid molecule, first subsequent mtHTT variant reference allele-specific/second subsequent mtHTT variant reference allele-specific inhibitory nucleic acid molecule, first subsequent mtHTT variant alternate allele-specific/second subsequent mtHTT variant reference allele-specific inhibitory nucleic acid molecule, first subsequent mtHTT variant reference allele-specific/second subsequent mtHTT variant alternate allele-specific inhibitory nucleic acid molecule, first further subsequent mtHTT variant alternate allele-specific/second further subsequent mtHTT variant alternate allele-specific inhibitory nucleic acid molecule, first further subsequent mtHTT variant reference allele-specific/second further subsequent mtHTT variant reference allele-specific inhibitory nucleic acid molecule, first further subsequent mtHTT variant alternate allele-specific/second further subsequent mtHTT variant reference allele-specific inhibitory nucleic acid molecule, and first further subsequent mtHTT variant reference allele-specific/second further subsequent mtHTT variant alternate allele-specific inhibitory nucleic acid molecule comprise an siRNA.

In any of the methods of treatment described herein, the first mtHTT variant alternate allele-specific inhibitory nucleic acid molecule, first subsequent mtHTT variant alternate allele-specific/second subsequent mtHTT variant alternate allele-specific inhibitory nucleic acid molecule, first subsequent mtHTT variant reference allele-specific/second subsequent mtHTT variant reference allele-specific inhibitory nucleic acid molecule, first subsequent mtHTT variant alternate allele-specific/second subsequent mtHTT variant reference allele-specific inhibitory nucleic acid molecule, first subsequent mtHTT variant reference allele-specific/second subsequent mtHTT variant alternate allele-specific inhibitory nucleic acid molecule, first further subsequent mtHTT variant alternate allele-specific/second further subsequent mtHTT variant alternate allele-specific inhibitory nucleic acid molecule, first further subsequent mtHTT variant reference allele-specific/second further subsequent mtHTT variant reference allele-specific inhibitory nucleic acid molecule, first further subsequent mtHTT variant alternate allele-specific/second further subsequent mtHTT variant reference allele-specific inhibitory nucleic acid molecule, and first further subsequent mtHTT variant reference allele-specific/second further subsequent mtHTT variant alternate allele-specific inhibitory nucleic acid molecule comprise an antisense nucleic acid molecule.

In any of the methods of treatment described herein, the first mtHTT variant comprises 4:3228683:AGAG:A; and i) the first mtHTT variant alternate-specific inhibitory nucleic acid molecule comprises an antisense nucleic acid molecule comprising the nucleotide sequence of SEQ ID NO:334, SEQ ID NO:335, or SEQ ID NO:336; or ii) the first mtHTT variant alternate-specific inhibitory nucleic acid molecule comprises an siRNA comprising the nucleotide sequences of SEQ ID NOs:89/334, SEQ ID NOs:90/335, or SEQ ID NOs:91/336.

In any of the methods of treatment described herein, the first subsequent mtHTT variant comprises 4:3240433:C:G and the second subsequent mtHTT variant comprises 4:3240421:C:G, and: i) the first subsequent mtHTT variant alternate-specific/second subsequent mtHTT variant alternate-specific inhibitory nucleic acid molecule or the first further subsequent mtHTT variant alternate-specific/second further subsequent mtHTT variant alternate-specific inhibitory nucleic acid molecule comprises: a) an antisense nucleic acid molecule comprising the nucleotide sequence of SEQ ID NO:394, SEQ ID NO:396, SEQ ID NO:397, SEQ ID NO:402, SEQ ID NO:404, or SEQ ID NO:408; or b) an siRNA comprising the nucleotide sequences of SEQ ID NOs:149/394, SEQ ID NOs:151/396, SEQ ID NOs:152/397, SEQ ID NOs:157/402, SEQ ID NOs:159/404, or SEQ ID NOs:163/408; ii) the first subsequent mtHTT variant reference-specific/second subsequent mtHTT reference-specific inhibitory nucleic acid molecule or the first further subsequent mtHTT variant reference-specific/second further subsequent mtHTT reference-specific inhibitory nucleic acid molecule comprises: a) an antisense nucleic acid molecule comprising the nucleotide sequence of SEQ ID NO:398 or SEQ ID NO:405; or b) an siRNA comprising the nucleotide sequences of SEQ ID NOs:153/398 or SEQ ID NOs:160/405; iii) the first subsequent mtHTT variant alternate-specific/second subsequent mtHTT variant reference-specific inhibitory nucleic acid molecule or the first further subsequent mtHTT variant alternate-specific/second further subsequent mtHTT variant reference-specific inhibitory nucleic acid molecule comprises: a) an antisense nucleic acid molecule comprising the nucleotide sequence of SEQ ID NO:395, SEQ ID NO:399, SEQ ID NO:401, SEQ ID NO:403, or SEQ ID NO:407; or b) an siRNA comprising the nucleotide sequences of SEQ ID NOs:150/395, SEQ ID NOs:154/399, SEQ ID NOs:156/401, SEQ ID NOs:158/403, or SEQ ID NOs:162/407; and iv) the first subsequent mtHTT variant reference-specific/second subsequent mtHTT variant alternate-specific inhibitory nucleic acid molecule or first further subsequent mtHTT variant reference-specific/second further subsequent mtHTT variant alternate-specific inhibitory nucleic acid molecule comprises: a) an antisense nucleic acid molecule comprising the nucleotide sequence of SEQ ID NO:400 or SEQ ID NO:406; or b) an siRNA comprising the nucleotide sequences of SEQ ID NOs:155/400 or SEQ ID NOs:161/406.

In some embodiments, the antisense nucleic acid molecule and siRNA hybridize to a sequence within the mtHTT genomic nucleic acid molecule or mRNA molecule and decreases expression of the mtHTT polypeptide in a cell in the subject.

In some embodiments, the antisense nucleic acid molecules comprise or consist of any of the nucleotide sequences represented by SEQ ID NOs:246-490. In some embodiments, the siRNA molecules comprise or consist of any of the nucleotide sequences represented by SEQ ID NOs:1-490 (e.g., the sense strand is, for example, SEQ ID NO:1 and the corresponding antisense strand is SEQ ID NO:246; the sense strand is, for example, SEQ ID NO:2 and the corresponding antisense strand is SEQ ID NO:247; the sense strand is, for example, SEQ ID NO:245 and the corresponding antisense strand is SEQ ID NO:490; etc.). Exemplary antisense nucleic acid molecules and siRNA molecules are shown in Table 2.

TABLE 2

| SEQ ID | | SEQ ID | Target Antisense | | Target |
|---|---|---|---|---|---|
| NO: | Sense Sequence | NO: | Sequence | Target SNP | Allele |
| 1 | GACGGCCGCTCAGG | 246 | AGCAGAACCUGAGCGGC | 3074723:C > T | REF |
| | TTCTGCT | | CGUC | | |
| 2 | GACGGCCGCTCAGG | 247 | AACAGAACCUGAGCGGC | 3074723:C > T | ALT |
| | TTCTGTT | | CGUC | | |
| 3 | ACGGCCGCTCAGGTT | 248 | AAGCAGAACCUGAGCGG | 3074723:C > T | REF |
| | CTGCTT | | CCGU | | |
| 4 | ACGGCCGCTCAGGTT | 249 | AAACAGAACCUGAGCGG | 3074723:C > T | ALT |
| | CTGTTT | | CCGU | | |
| 5 | CGGCCGCTCAGGTTC | 250 | AAAGCAGAACCUGAGCG | 3074723:C > T | REF |
| | TGCTTT | | GCCG | | |
| 6 | CGGCCGCTCAGGTTC | 251 | AAAACAGAACCUGAGCG | 3074723:C > T | ALT |
| | TGTTTT | | GCCG | | |
| 7 | GGCCGCTCAGGTTCT | 252 | AAAAGCAGAACCUGAGC | 3074723:C > T | REF |
| | GCTTTT | | GGCC | | |
| 8 | GGCCGCTCAGGTTCT | 253 | AAAAACAGAACCUGAGC | 3074723:C > T | ALT |
| | GTTTTT | | GGCC | | |
| 9 | GCCGCTCAGGTTCTG | 254 | UAAAAGCAGAACCUGAG | 3074723:C > T | REF |
| | CTTTTA | | CGGC | | |
| 10 | GCCGCTCAGGTTCTG | 255 | UAAAAACAGAACCUGAG | 3074723:C > T | ALT |
| | TTTTTA | | CGGC | | |
| 11 | CCGCTCAGGTTCTGC | 256 | GUAAAAGCAGAACCUGA | 3074723:C > T | REF |
| | TTTTAC | | GCGG | | |
| 12 | CCGCTCAGGTTCTGT | 257 | GUAAAAACAGAACCUGA | 3074723:C > T | ALT |
| | TTTTAC | | GCGG | | |
| 13 | CGCTCAGGTTCTGCT | 258 | GGUAAAAGCAGAACCUG | 3074723:C > T | REF |
| | TTTACC | | AGCG | | |
| 14 | CGCTCAGGTTCTGTT | 259 | GGUAAAAACAGAACCUG | 3074723:C > T | ALT |
| | TTTACC | | AGCG | | |
| 15 | GCTCAGGTTCTGCTT | 260 | AGGUAAAAGCAGAACCU | 3074723:C > T | REF |
| | TTACCT | | GAGC | | |
| 16 | GCTCAGGTTCTGTTT | 261 | AGGUAAAAACAGAACCU | 3074723:C > T | ALT |
| | TTACCT | | GAGC | | |
| 17 | CTCAGGTTCTGCTTTT | 262 | CAGGUAAAAGCAGAACC | 3074723:C > T | REF |
| | ACCTG | | UGAG | | |
| 18 | CTCAGGTTCTGTTTTT | 263 | CAGGUAAAAACAGAACC | 3074723:C > T | ALT |
| | ACCTG | | UGAG | | |
| 19 | TGGCTACTAAATGTG | 264 | UAAGAGCACAUUUAGU | 3115441:C > G | REF |
| | CTCTTA | | AGCCA | | |
| 20 | TGGCTACTAAATGTG | 265 | UAACAGCACAUUUAGU | 3115441:C > G | ALT |
| | CTGTTA | | AGCCA | | |
| 21 | GGCTACTAAATGTGC | 266 | CUAACAGCACAUUUAGU | 3115441:C > G | ALT |
| | TGTTAG | | AGCC | | |
| 22 | GCTACTAAATGTGCT | 267 | CCUAAGAGCACAUUUAG | 3115441:C > G | REF |
| | CTTAGG | | UAGC | | |
| 23 | GCTACTAAATGTGCT | 268 | CCUAACAGCACAUUUAG | 3115441:C > G | ALT |
| | GTTAGG | | UAGC | | |
| 24 | CTACTAAATGTGCTC | 269 | GCCUAAGAGCACAUUUA | 3115441:C > G | REF |
| | TTAGGC | | GUAG | | |
| 25 | CTACTAAATGTGCTG | 270 | GCCUAACAGCACAUUUA | 3115441:C > G | ALT |
| | TTAGGC | | GUAG | | |
| 26 | TACTAAATGTGCTCT | 271 | AGCCUAAGAGCACAUUU | 3115441:C > G | REF |
| | TAGGCT | | AGUA | | |
| 27 | TACTAAATGTGCTGT | 272 | AGCCUAACAGCACAUUU | 3115441:C > G | ALT |
| | TAGGCT | | AGUA | | |
| 28 | ACTAAATGTGCTCTT | 273 | AAGCCUAAGAGCACAUU | 3115441:C > G | REF |
| | AGGCTT | | UAGU | | |
| 29 | ACTAAATGTGCTGTT | 274 | AAGCCUAACAGCACAUU | 3115441:C > G | ALT |
| | AGGCTT | | UAGU | | |
| 30 | CTAAATGTGCTCTTA | 275 | UAAGCCUAAGAGCACAU | 3115441:C > G | REF |
| | GGCTTA | | UUAG | | |
| 31 | CTAAATGTGCTGTTA | 276 | UAAGCCUAACAGCACAU | 3115441:C > G | ALT |
| | GGCTTA | | UUAG | | |
| 32 | TAAATGTGCTCTTAG | 277 | GUAAGCCUAAGAGCACA | 3115441:C > G | REF |
| | GCTTAC | | UUUA | | |
| 33 | TAAATGTGCTGTTAG | 278 | GUAAGCCUAACAGCACA | 3115441:C > G | ALT |
| | GCTTAC | | UUUA | | |
| 34 | AAAAGTTTGGAGGG | 279 | GAAGAAACCCUCCAAAC | 3160329:C > T | ALT |
| | TTTCTTC | | UUUU | | |
| 35 | AGTTTGGAGGGTTTC | 280 | AGCGAAGAAACCCUCCA | 3160329:C > T | ALT |
| | TTCGCT | | AACU | | |
| 36 | GTTTGGAGGGTTTCT | 281 | GAGCGGAGAAACCCUCC | 3160329:C > T | REF |
| | CCGCTC | | AAAC | | |
| 37 | GTTTGGAGGGTTTCT | 282 | GAGCGAAGAAACCCUCC | 3160329:C > T | ALT |
| | TCGCTC | | AAAC | | |
| 38 | TTTGGAGGGTTTCTC | 283 | UGAGCGGAGAAACCCUC | 3160329:C > T | REF |
| | CGCTCA | | CAAA | | |
| 39 | TTTGGAGGGTTTCTT | 284 | UGAGCGAAGAAACCCUC | 3160329:C > T | ALT |
| | CGCTCA | | CAAA | | |
| 40 | TTGGAGGGTTTCTCC | 285 | CUGAGCGGAGAAACCCU | 3160329:C > T | REF |
| | GCTCAG | | CCAA | | |
| 41 | TTGGAGGGTTTCTTC | 286 | CUGAGCGAAGAAACCCU | 3160329:C > T | ALT |
| | GCTCAG | | CCAA | | |
| 42 | TGGAGGGTTTCTTCG | 287 | GCUGAGCGAAGAAACCC | 3160329:C > T | ALT |
| | CTCAGC | | UCCA | | |
| 43 | TGGAGGGTTTCTCCG | 288 | GCUGAGCGGAGAAACCC | 3160329:C > T | REF |
| | CTCAGC | | UCCA | | |
| 44 | AGATGGGGACAGTA | 289 | GUUGAAUUACUGUCCC | 3187820:C > A | ALT |
| | ATTCAAC | | CAUCU | | |
| 45 | AGATGGGGACAGTA | 290 | GUUGAAGUACUGUCCC | 3187820:C > A | REF |
| | CTTCAAC | | CAUCU | | |
| 46 | GATGGGGACAGTAA | 291 | CGUUGAAUUACUGUCC | 3187820:C > A | ALT |
| | TTCAACG | | CCAUC | | |
| 47 | GATGGGGACAGTAC | 292 | CGUUGAAGUACUGUCC | 3187820:C > A | REF |
| | TTCAACG | | CCAUC | | |
| 48 | ATGGGGACAGTACTT | 293 | GCGUUGAAGUACUGUC | 3187820:C > A | REF |
| | CAACGC | | CCCAU | | |
| 49 | ATGGGGACAGTAATT | 294 | GCGUUGAAUUACUGUC | 3187820:C > A | ALT |
| | CAACGC | | CCCAU | | |
| 50 | TGGGGACAGTACTTC | 295 | AGCGUUGAAGUACUGU | 3187820:C > A | REF |
| | AACGCT | | CCCCA | | |
| 51 | TGGGGACAGTAATTC | 296 | AGCGUUGAAUUACUGU | 3187820:C > A | ALT |
| | AACGCT | | CCCCA | | |
| 52 | GGGGACAGTAATTC | 297 | UAGCGUUGAAUUACUG | 3187820:C > A | ALT |
| | AACGCTA | | UCCCC | | |
| 53 | GGGGACAGTACTTCA | 298 | UAGCGUUGAAGUACUG | 3187820:C > A | REF |
| | ACGCTA | | UCCCC | | |
| 54 | CGAGCTGCCTGCAGA | 299 | GCCGGCUCUGCAGGCAG | 3212105:G > A | REF |
| | GCCGGC | | CUCG | | |
| 55 | GAGCTGCCTGCAGA | 300 | CGCCGGCUCUGCAGGCA | 3212105:G > A | REF |
| | GCCGGCG | | GCUC | | |
| 56 | AGCTGCCTGCAGAGC | 301 | CCGCCGGCUCUGCAGGC | 3212105:G > A | REF |
| | CGGCGG | | AGCU | | |
| 57 | GCTGCCTGCAGAGCC | 302 | GCCGCCGGCUCUGCAGG | 3212105:G > A | REF |
| | GGCGGC | | CAGC | | |
| 58 | TGCCTGCAGAGCCG | 303 | AGGCCGCCGGCUCUGCA | 3212105:G > A | REF |
| | GCGGCCT | | GGCA | | |
| 59 | CCACGCCTGCTCCCT | 304 | UAGAUGAGGGAGCAGG | 3214108:T > C | REF |
| | CATCTA | | CGUGG | | |
| 60 | CACGCCTGCTCCCTC | 305 | GUGGAUGAGGGAGCAG | 3214108:T > C | ALT |
| | ATCCAC | | GCGUG | | |
| 61 | CACGCCTGCTCCCTC | 306 | GUAGAUGAGGGAGCAG | 3214108:T > C | REF |
| | ATCTAC | | GCGUG | | |
| 62 | ACGCCTGCTCCCTCA | 307 | AGUGGAUGAGGGAGCA | 3214108:T > C | ALT |
| | TCCACT | | GGCGU | | |
| 63 | CGCCTGCTCCCTCAT | 308 | CAGUGGAUGAGGGAGC | 3214108:T > C | ALT |
| | CCACTG | | AGGCG | | |
| 64 | GCTCCCTCATCTACT | 309 | GCACACAGUAGAUGAG | 3214108:T > C | REF |
| | GTGTGC | | GGAGC | | |
| 65 | GCTCCCTCATCCACT | 310 | GCACACAGUGGAUGAG | 3214108:T > C | ALT |
| | GTGTGC | | GGAGC | | |
| 66 | CTCCCTCATCTACTGT | 311 | UGCACACAGUAGAUGA | 3214108:T > C | REF |
| | GTGCA | | GGGAG | | |
| 67 | AGCGCAACCAGTTTG | 312 | UCAGCUCAAACUGGUU | 3229934:G > A | REF |
| | AGCTGA | | GCGCU | | |
| 68 | AGCGCAACCAGTTTG | 313 | UUAGCUCAAACUGGUU | 3229934:G > A | ALT |
| | AGCTAA | | GCGCU | | |
| 69 | GCGCAACCAGTTTGA | 314 | AUCAGCUCAAACUGGU | 3229934:G > A | REF |
| | GCTGAT | | UGCGC | | |
| 70 | GCGCAACCAGTTTGA | 315 | AUUAGCUCAAACUGGU | 3229934:G > A | ALT |
| | GCTAAT | | UGCGC | | |
| 71 | CGCAACCAGTTTGAG | 316 | CAUUAGCUCAAACUGG | 3229934:G > A | ALT |
| | CTAATG | | UUGCG | | |
| 72 | GCAACCAGTTTGAGC | 317 | ACAUUAGCUCAAACUGG | 3229934:G > A | ALT |
| | TAATGT | | UUGC | | |
| 73 | AACCAGTTTGAGCTG | 318 | AUACAUCAGCUCAAACU | 3229934:G > A | REF |
| | ATGTAT | | GGUU | | |
| 74 | ACCAGTTTGAGCTGA | 319 | CAUACAUCAGCUCAAAC | 3229934:G > A | REF |
| | TGTATG | | UGGU | | |
| 75 | ACCAGTTTGAGCTAA | 320 | CAUACAUUAGCUCAAAC | 3229934:G > A | ALT |
| | TGTATG | | UGGU | | |
| 76 | CCAGTTTGAGCTGAT | 321 | ACAUACAUCAGCUCAAA | 3229934:G > A | REF |
| | GTATGT | | CUGG | | |
| 77 | CCAGTTTGAGCTAAT | 322 | ACAUACAUUAGCUCAAA | 3229934:G > A | ALT |
| | GTATGT | | CUGG | | |
| 78 | CAGTTTGAGCTGATG | 323 | CACAUACAUCAGCUCAA | 3229934:G > A | REF |
| | TATGTG | | ACUG | | |
| 79 | AGTTTGAGCTGATGT | 324 | UCACAUACAUCAGCUCA | 3229934:G > A | REF |
| | ATGTGA | | AACU | | |
| 80 | AGTTTGAGCTAATGT | 325 | UCACAUACAUUAGCUCA | 3229934:G > A | ALT |
| | ATGTGA | | AACU | | |
| 81 | AGAAGCTGCTGCTGC | 326 | UGAUCUGCAGCAGCAGC | 3225692:A > G | ALT |
| | AGATCA | | UUCU | | |
| 82 | AGAAGCTGCTGCTAC | 327 | UGAUCUGUAGCAGCAG | 3225692:A > G | REF |
| | AGATCA | | CUUCU | | |
| 83 | GAAGCTGCTGCTGCA | 328 | UUGAUCUGCAGCAGCA | 3225692:A > G | ALT |
| | GATCAA | | GCUUC | | |
| 84 | GAAGCTGCTGCTACA | 329 | UUGAUCUGUAGCAGCA | 3225692:A > G | REF |
| | GATCAA | | GCUUC | | |
| 85 | AAGCTGCTGCTGCAG | 330 | GUUGAUCUGCAGCAGC | 3225692:A > G | ALT |
| | ATCAAC | | AGCUU | | |
| 86 | AAGCTGCTGCTACAG | 331 | GUUGAUCUGUAGCAGC | 3225692:A > G | REF |
| | ATCAAC | | AGCUU | | |
| 87 | AGCTGCTGCTACAGA | 332 | GGUUGAUCUGUAGCAG | 3225692:A > G | REF |
| | TCAACC | | CAGCU | | |
| 88 | GAGGAATGGGACGA | 333 | CUCUUCCUCGUCCCAUU | 3228683:AGAG > | REF |
| | GGAAGAG | | CCUC | A | |
| 89 | AGGAAGAGGAGGAG | 334 | CGUCGGCCUCCUCCUCU | 3228683:AGAG > | ALT |
| | GCCGACG | | UCCU | A | |
| 90 | GGAAGAGGAGGAG | 335 | GCGUCGGCCUCCUCCUC | 3228683:AGAG > | ALT |
| | GCCGACGC | | UUCC | A | |
| 91 | GAAGAGGAGGAGGC | 336 | GGCGUCGGCCUCCUCCU | 3228683:AGAG > | ALT |
| | CGACGCC | | CUUC | A | |
| 92 | GGTTGGAGCCCTGCA | 337 | ACGCCGUGCAGGGCUCC | 3233253:G > A | REF |
| | CGGCGT | | AACC | | |
| 93 | GGTTGGAGCCCTGCA | 338 | AUGCCGUGCAGGGCUCC | 3233253:G > A | ALT |
| | CGGCAT | | AACC | | |
| 94 | GTTGGAGCCCTGCAC | 339 | GAUGCCGUGCAGGGCU | 3233253:G > A | ALT |
| | GGCATC | | CCAAC | | |
| 95 | GTTGGAGCCCTGCAC | 340 | GACGCCGUGCAGGGCUC | 3233253:G > A | REF |
| | GGCGTC | | CAAC | | |
| 96 | TTGGAGCCCTGCACG | 341 | GGAUGCCGUGCAGGGC | 3233253:G > A | ALT |
| | GCATCC | | UCCAA | | |
| 97 | TTGGAGCCCTGCACG | 342 | GGACGCCGUGCAGGGC | 3233253:G > A | REF |
| | GCGTCC | | UCCAA | | |
| 98 | TGGAGCCCTGCACG | 343 | AGGACGCCGUGCAGGGC | 3233253:G > A | REF |
| | GCGTCCT | | UCCA | | |
| 99 | GGAGCCCTGCACGG | 344 | GAGGACGCCGUGCAGG | 3233253:G > A | REF |
| | CGTCCTC | | GCUCC | | |
| 100 | GGAGCCCTGCACGG | 345 | GAGGAUGCCGUGCAGG | 3233253:G > A | ALT |
| | CATCCTC | | GCUCC | | |
| 101 | GAGCCCTGCACGGC | 346 | AGAGGAUGCCGUGCAG | 3233253:G > A | ALT |
| | ATCCTCT | | GGCUC | | |
| 102 | GAGCCCTGCACGGC | 347 | AGAGGACGCCGUGCAG | 3233253:G > A | REF |
| | GTCCTCT | | GGCUC | | |
| 103 | AGCCCTGCACGGCGT | 348 | UAGAGGACGCCGUGCA | 3233253:G > A | REF |
| | CCTCTA | | GGGCU | | |
| 104 | AGCCCTGCACGGCAT | 349 | UAGAGGAUGCCGUGCA | 3233253:G > A | ALT |
| | CCTCTA | | GGGCU | | |
| 105 | CCTGCACGGCGTCCT | 350 | ACAUAGAGGACGCCGU | 3233253:G > A | REF |
| | CTATGT | | GCAGG | | |
| 106 | CCTGCACGGCATCCT | 351 | ACAUAGAGGAUGCCGU | 3233253:G > A | ALT |
| | CTATGT | | GCAGG | | |
| 107 | GGAGCCTTTGGAAGT | 352 | GCACAGACUUCCAAAGG | 3240118:C > T | ALT |
| | CTGTGC | | CUCC | | |
| 108 | GGAGCCTTTGGAAGT | 353 | GCGCAGACUUCCAAAGG | 3240118:C > T | REF |
| | CTGCGC | | CUCC | | |
| 109 | GAGCCTTTGGAAGTC | 354 | GGCACAGACUUCCAAAG | 3240118:C > T | ALT |
| | TGTGCC | | GCUC | | |
| 110 | GAGCCTTTGGAAGTC | 355 | GGCGCAGACUUCCAAAG | 3240118:C > T | REF |
| | TGCGCC | | GCUC | | |
| 111 | AGCCTTTGGAAGTCT | 356 | GGGCACAGACUUCCAAA | 3240118:C > T | ALT |
| | GTGCCC | | GGCU | | |
| 112 | AGCCTTTGGAAGTCT | 357 | GGGCGCAGACUUCCAAA | 3240118:C > T | REF |
| | GCGCCC | | GGCU | | |
| 113 | GCCTTTGGAAGTCTG | 358 | AGGGCGCAGACUUCCAA | 3240118:C > T | REF |
| | CGCCCT | | AGGC | | |
| 114 | CCTTTGGAAGTCTGC | 359 | AAGGGCGCAGACUUCCA | 3240118:C > T | REF |
| | GCCCTT | | AAGG | | |
| 115 | CCTTTGGAAGTCTGT | 360 | AAGGGCACAGACUUCCA | 3240118:C > T | ALT |
| | GCCCTT | | AAGG | | |
| 116 | CTTTGGAAGTCTGTG | 361 | CAAGGGCACAGACUUCC | 3240118:C > T | ALT |
| | CCCTTG | | AAAG | | |
| 117 | CTTTGGAAGTCTGCG | 362 | CAAGGGCGCAGACUUCC | 3240118:C > T | REF |
| | CCCTTG | | AAAG | | |
| 118 | TTTGGAAGTCTGTGC | 363 | ACAAGGGCACAGACUUC | 3240118:C > T | ALT |
| | CCTTGT | | CAAA | | |
| 119 | TTTGGAAGTCTGCGC | 364 | ACAAGGGCGCAGACUUC | 3240118:C > T | REF |
| | CCTTGT | | CAAA | | |
| 120 | TTGGAAGTCTGTGCC | 365 | CACAAGGGCACAGACUU | 3240118:C > T | ALT |
| | CTTGTG | | CCAA | | |
| 121 | TTGGAAGTCTGCGCC | 366 | CACAAGGGCGCAGACUU | 3240118:C > T | REF |
| | CTTGTG | | CCAA | | |
| 122 | TGGAAGTCTGCGCCC | 367 | GCACAAGGGCGCAGACU | 3240118:C > T | REF |
| | TTGTGC | | UCCA | | |
| 123 | TGGAAGTCTGTGCCC | 368 | GCACAAGGGCACAGACU | 3240118:C > T | ALT |
| | TTGTGC | | UCCA | | |
| 124 | GCTGGTTGTTGCCAG | 369 | UGCAACCUGGCAACAAC | 3240373:G > A | REF |
| | GTTGCA | | CAGC | | |
| 125 | GCTGGTTGTTGCCAG | 370 | UGUAACCUGGCAACAAC | 3240373:G > A | ALT |
| | GTTACA | | CAGC | | |
| 126 | CTGGTTGTTGCCAGG | 371 | CUGCAACCUGGCAACAA | 3240373:G > A | REF |
| | TTGCAG | | CCAG | | |
| 127 | CTGGTTGTTGCCAGG | 372 | CUGUAACCUGGCAACAA | 3240373:G > A | ALT |
| | TTACAG | | CCAG | | |
| 128 | TGGTTGTTGCCAGGT | 373 | GCUGUAACCUGGCAACA | 3240373:G > A | ALT |
| | TACAGC | | ACCA | | |
| 129 | TGGTTGTTGCCAGGT | 374 | GCUGCAACCUGGCAACA | 3240373:G > A | REF |
| | TGCAGC | | ACCA | | |
| 130 | GGTTGTTGCCAGGTT | 375 | AGCUGCAACCUGGCAAC | 3240373:G > A | REF |
| | GCAGCT | | AACC | | |
| 131 | GGTTGTTGCCAGGTT | 376 | AGCUGUAACCUGGCAAC | 3240373:G > A | ALT |
| | ACAGCT | | AACC | | |
| 132 | GTTGTTGCCAGGTTA | 377 | CAGCUGUAACCUGGCAA | 3240373:G > A | ALT |
| | CAGCTG | | CAAC | | |
| 133 | TGTTGCCAGGTTACA | 378 | AGCAGCUGUAACCUGGC | 3240373:G > A | ALT |
| | GCTGCT | | AACA | | |
| 134 | CCTCCCTCCTGCAGG | 379 | AGCCAGCCUGCAGGAGG | 3240421:C > G | REF |
| | CTGGCT | | GAGG | | |
| 135 | CCTCCCTCCTGCAGG | 380 | ACCCAGCCUGCAGGAGG | 3240421:C > G | ALT |
| | CTGGGT | | GAGG | | |
| 136 | CTCCCTCCTGCAGGC | 381 | CAGCCAGCCUGCAGGAG | 3240421:C > G | REF |
| | TGGCTG | | GGAG | | |
| 137 | CTCCCTCCTGCAGGC | 382 | CACCCAGCCUGCAGGAG | 3240421:C > G | ALT |
| | TGGGTG | | GGAG | | |
| 138 | TCCCTCCTGCAGGCT | 383 | ACAGCCAGCCUGCAGGA | 3240421:C > G | REF |
| | GGCTGT | | GGGA | | |
| 139 | CCCTCCTGCAGGCTG | 384 | AACACCCAGCCUGCAGG | 3240421:C > G | ALT |
| | GGTGTT | | AGGG | | |
| 140 | CCCTCCTGCAGGCTG | 385 | AACAGCCAGCCUGCAGG | 3240421:C > G | REF |
| | GCTGTT | | AGGG | | |
| 141 | CCTCCTGCAGGCTGG | 386 | CAACAGCCAGCCUGCAG | 3240421:C > G | REF |
| | CTGTTG | | GAGG | | |
| 142 | CCTCCTGCAGGCTGG | 387 | CAACACCCAGCCUGCAG | 3240421:C > G | ALT |
| | GTGTTG | | GAGG | | |
| 143 | CTCCTGCAGGCTGGC | 388 | CCAACAGCCAGCCUGCA | 3240421:C > G | REF |
| | TGTTGG | | GGAG | | |
| 144 | CTCCTGCAGGCTGGG | 389 | CCAACACCCAGCCUGCA | 3240421:C > G | ALT |
| | TGTTGG | | GGAG | | |
| 145 | TCCTGCAGGCTGGCT | 390 | GCCAACAGCCAGCCUGC | 3240421:C > G | REF |
| | GTTGGC | | AGGA | | |
| 146 | CCTGCAGGCTGGCTG | 391 | GGCCAACAGCCAGCCUG | 3240421:C > G | REF |
| | TTGGCC | | CAGG | | |
| 147 | TGCAGGCTGGGTGTT | 392 | GGGGCCAACACCCAGCC | 3240421:C > G | ALT |
| | GGCCCC | | UGCA | | |
| 148 | TGCAGGCTGGCTGTT | 393 | GGGGCCAACAGCCAGCC | 3240421:C > G | REF |
| | GGCCCC | | UGCA | | |
| 149 | AGGCTGGGTGTTGG | 394 | ACAGGGGCCAACACCCA | 3240433:C > G, | ALT,ALT |
| | CCCCTGT | | GCCU | 3240421:C > G | |
| 150 | GGCTGGCTGTTGGCC | 395 | CACAGGGGCCAACAGCC | 3240433:C > G, | ALT, REF |
| | CCTGTG | | AGCC | 3240421:C > G | |
| 151 | GGCTGGGTGTTGGC | 396 | CACAGGGGCCAACACCC | 3240433:C > G, | ALT, ALT |
| | CCCTGTG | | AGCC | 3240421:C > G | |
| 152 | GCTGGGTGTTGGCCC | 397 | GCACAGGGGCCAACACC | 3240433:C > G, | ALT, ALT |
| | CTGTGC | | CAGC | 3240421:C > G | |
| 153 | GCTGGCTGTTGGCCC | 398 | GCAGAGGGGCCAACAGC | 3240433:C > G, | REF, REF |
| | CTCTGC | | CAGC | 3240421:C > G | |
| 154 | GCTGGCTGTTGGCCC | 399 | GCACAGGGGCCAACAGC | 3240433:C > G, | ALT, REF |
| | CTGTGC | | CAGC | 3240421:C > G | |
| 155 | GCTGGGTGTTGGCCC | 400 | GCAGAGGGGCCAACACC | 3240433:C > G, | REF, ALT |
| | CTCTGC | | CAGC | 3240421:C > G | |
| 156 | CTGGCTGTTGGCCCC | 401 | AGCACAGGGGCCAACAG | 3240433:C > G, | ALT, REF |
| | TGTGCT | | CCAG | 3240421:C > G | |
| 157 | CTGGGTGTTGGCCCC | 402 | AGCACAGGGGCCAACAC | 3240433:C > G, | ALT, ALT |
| | TGTGCT | | CCAG | 3240421:C > G | |
| 158 | TGGCTGTTGGCCCCT | 403 | CAGCACAGGGGCCAACA | 3240433:C > G, | ALT, REF |
| | GTGCTG | | GCCA | 3240421:C > G | |
| 159 | TGGGTGTTGGCCCCT | 404 | CAGCACAGGGGCCAACA | 3240433:C > G, | ALT, ALT |
| | GTGCTG | | CCCA | 3240421:C > G | |
| 160 | CTGTTGGCCCCTCTG | 405 | GGACAGCAGAGGGGCCA | 3240433:C > G, | REF, REF |
| | CTGTCC | | ACAG | 3240421:C > G | |
| 161 | GTGTTGGCCCCTCTG | 406 | GGACAGCAGAGGGGCCA | 3240433:C > G, | REF, ALT |
| | CTGTCC | | ACAC | 3240421:C > G | |
| 162 | CTGTTGGCCCCTGTG | 407 | GGACAGCACAGGGGCCA | 3240433:C > G, | ALT, REF |
| | CTGTCC | | ACAG | 3240421:C > G | |
| 163 | GTGTTGGCCCCTGTG | 408 | GGACAGCACAGGGGCCA | 3240433:C > G, | ALT, ALT |
| | CTGTCC | | ACAC | 3240421:C > G | |
| 164 | TGTTGGCCCCTCTGC | 409 | AGGACAGCAGAGGGGCC | 3240433:C > G | REF |
| | TGTCCT | | AACA | | |
| 165 | TGTTGGCCCCTGTGC | 410 | AGGACAGCACAGGGGCC | 3240433:C > G | ALT |
| | TGTCCT | | AACA | | |
| 166 | GTTGGCCCCTGTGCT | 411 | CAGGACAGCACAGGGGC | 3240433:C > G | ALT |
| | GTCCTG | | CAAC | | |
| 167 | GTTGGCCCCTCTGCT | 412 | CAGGACAGCAGAGGGGC | 3240433:C > G | REF |
| | GTCCTG | | CAAC | | |
| 168 | GATGCACAGATGCCA | 413 | AUGCCAUGGCAUCUGU | 3240545:C > A | ALT |
| | TGGCAT | | GCAUC | | |
| 169 | ATGCACAGATGCCAT | 414 | CAGGCCAUGGCAUCUG | 3240545:C > A | REF |
| | GGCCTG | | UGCAU | | |
| 170 | ATGCACAGATGCCAT | 415 | CAUGCCAUGGCAUCUG | 3240545:C > A | ALT |
| | GGCATG | | UGCAU | | |
| 171 | TGCACAGATGCCATG | 416 | ACAGGCCAUGGCAUCUG | 3240545:C > A | REF |
| | GCCTGT | | UGCA | | |
| 172 | TGCACAGATGCCATG | 417 | ACAUGCCAUGGCAUCUG | 3240545:C > A | ALT |
| | GCATGT | | UGCA | | |
| 173 | GCACAGATGCCATG | 418 | CACAGGCCAUGGCAUCU | 3240545:C > A | REF |
| | GCCTGTG | | GUGC | | |
| 174 | GCACAGATGCCATG | 419 | CACAUGCCAUGGCAUCU | 3240545:C > A | ALT |
| | GCATGTG | | GUGC | | |
| 175 | CACAGATGCCATGGC | 420 | GCACAGGCCAUGGCAUC | 3240545:C > A | REF |
| | CTGTGC | | UGUG | | |
| 176 | CACAGATGCCATGGC | 421 | GCACAUGCCAUGGCAUC | 3240545:C > A | ALT |
| | ATGTGC | | UGUG | | |
| 177 | ACAGATGCCATGGCA | 422 | AGCACAUGCCAUGGCAU | 3240545:C > A | ALT |
| | TGTGCT | | CUGU | | |
| 178 | CAGATGCCATGGCCT | 423 | CAGCACAGGCCAUGGCA | 3240545:C > A | REF |
| | GTGCTG | | UCUG | | |
| 179 | CAGATGCCATGGCAT | 424 | CAGCACAUGCCAUGGCA | 3240545:C > A | ALT |
| | GTGCTG | | UCUG | | |
| 180 | GCTGGGGGTGCTAG | 425 | CGGGUGUCUAGCACCCC | 3240580:C > T | REF |
| | ACACCCG | | CAGC | | |
| 181 | GCTGGGGGTGCTAG | 426 | CAGGUGUCUAGCACCCC | 3240580:C > T | ALT |
| | ACACCTG | | CAGC | | |
| 182 | CTGGGGGTGCTAGA | 427 | CCGGGUGUCUAGCACCC | 3240580:C > T | REF |
| | CACCCGG | | CCAG | | |
| 183 | TGGGGGTGCTAGAC | 428 | GCCGGGUGUCUAGCACC | 3240580:C > T | REF |
| | ACCCGGC | | CCCA | | |
| 184 | GGGGGTGCTAGACA | 429 | UGCCGGGUGUCUAGCA | 3240580:C > T | REF |
| | CCCGGCA | | CCCCC | | |
| 185 | GGGGGTGCTAGACA | 430 | UGCCAGGUGUCUAGCA | 3240580:C > T | ALT |
| | CCTGGCA | | CCCCC | | |
| 186 | GGGGTGCTAGACAC | 431 | GUGCCGGGUGUCUAGC | 3240580:C > T | REF |
| | CCGGCAC | | ACCCC | | |
| 187 | GGGGTGCTAGACAC | 432 | GUGCCAGGUGUCUAGC | 3240580:C > T | ALT |
| | CTGGCAC | | ACCCC | | |
| 188 | GGGTGCTAGACACCT | 433 | GGUGCCAGGUGUCUAG | 3240580:C > T | ALT |
| | GGCACC | | CACCC | | |
| 189 | GGGTGCTAGACACCC | 434 | GGUGCCGGGUGUCUAG | 3240580:C > T | REF |
| | GGCACC | | CACCC | | |
| 190 | GGTGCTAGACACCCG | 435 | UGGUGCCGGGUGUCUA | 3240580:C > T | REF |
| | GCACCA | | GCACC | | |
| 191 | GGTGCTAGACACCTG | 436 | UGGUGCCAGGUGUCUA | 3240580:C > T | ALT |
| | GCACCA | | GCACC | | |
| 192 | GTGCTAGACACCCGG | 437 | AUGGUGCCGGGUGUCU | 3240580:C > T | REF |
| | CACCAT | | AGCAC | | |
| 193 | GTGCTAGACACCTGG | 438 | AUGGUGCCAGGUGUCU | 3240580:C > T | ALT |
| | CACCAT | | AGCAC | | |
| 194 | TGCTAGACACCTGGC | 439 | AAUGGUGCCAGGUGUC | 3240580:C > T | ALT |
| | ACCATT | | UAGCA | | |
| 195 | TGCTAGACACCCGGC | 440 | AAUGGUGCCGGGUGUC | 3240580:C > T | REF |
| | ACCATT | | UAGCA | | |
| 196 | GCTAGACACCTGGCA | 441 | GAAUGGUGCCAGGUGU | 3240580:C > T | ALT |
| | CCATTC | | CUAGC | | |
| 197 | GCTAGACACCCGGCA | 442 | GAAUGGUGCCGGGUGU | 3240580:C > T | REF |
| | CCATTC | | CUAGC | | |
| 198 | ATTTGGGAGCTCTGC | 443 | CAGCAAGCAGAGCUCCC | 3241491:C > T | ALT |
| | TTGCTG | | AAAU | | |
| 199 | TTGGGAGCTCTGCTT | 444 | GUCGGCAAGCAGAGCUC | 3241491:C > T | REF |
| | GCCGAC | | CCAA | | |
| 200 | TTGGGAGCTCTGCTT | 445 | GUCAGCAAGCAGAGCUC | 3241491:C > T | ALT |
| | GCTGAC | | CCAA | | |
| 201 | TGGGAGCTCTGCTTG | 446 | AGUCAGCAAGCAGAGCU | 3241491:C > T | ALT |
| | CTGACT | | CCCA | | |
| 202 | TGGGAGCTCTGCTTG | 447 | AGUCGGCAAGCAGAGC | 3241491:C > T | REF |
| | CCGACT | | UCCCA | | |
| 203 | GGGAGCTCTGCTTGC | 448 | CAGUCGGCAAGCAGAGC | 3241491:C > T | REF |
| | CGACTG | | UCCC | | |
| 204 | GGGAGCTCTGCTTGC | 449 | CAGUCAGCAAGCAGAGC | 3241491:C > T | ALT |
| | TGACTG | | UCCC | | |
| 205 | GGAGCTCTGCTTGCT | 450 | CCAGUCAGCAAGCAGAG | 3241491:C > T | ALT |
| | GACTGG | | CUCC | | |
| 206 | GGAGCTCTGCTTGCC | 451 | CCAGUCGGCAAGCAGAG | 3241491:C > T | REF |
| | GACTGG | | CUCC | | |
| 207 | GAGCTCTGCTTGCTG | 452 | GCCAGUCAGCAAGCAGA | 3241491:C > T | ALT |
| | ACTGGC | | GCUC | | |
| 208 | GAGCTCTGCTTGCCG | 453 | GCCAGUCGGCAAGCAGA | 3241491:C > T | REF |
| | ACTGGC | | GCUC | | |
| 209 | AGCTCTGCTTGCTGA | 454 | AGCCAGUCAGCAAGCAG | 3241491:C > T | ALT |
| | CTGGCT | | AGCU | | |
| 210 | AGCTCTGCTTGCCGA | 455 | AGCCAGUCGGCAAGCAG | 3241491:C > T | REF |
| | CTGGCT | | AGCU | | |
| 211 | GCTCTGCTTGCTGAC | 456 | CAGCCAGUCAGCAAGCA | 3241491:C > T | ALT |
| | TGGCTG | | GAGC | | |
| 212 | GCTCTGCTTGCCGAC | 457 | CAGCCAGUCGGCAAGCA | 3241491:C > T | REF |
| | TGGCTG | | GAGC | | |
| 213 | CTCTGCTTGCTGACT | 458 | ACAGCCAGUCAGCAAGC | 3241491:C > T | ALT |
| | GGCTGT | | AGAG | | |
| 214 | CTCTGCTTGCCGACT | 459 | ACAGCCAGUCGGCAAGC | 3241491:C > T | REF |
| | GGCTGT | | AGAG | | |
| 215 | CCAGGCAAGGTTGG | 460 | AUAGUCGCCAACCUUGC | 3241563:G > A | ALT |
| | CGACTAT | | CUGG | | |
| 216 | CCAGGCAAGGTTGG | 461 | ACAGUCGCCAACCUUGC | 3241563:G > A | REF |
| | CGACTGT | | CUGG | | |
| 217 | CAGGCAAGGTTGGC | 462 | GACAGUCGCCAACCUUG | 3241563:G > A | REF |
| | GACTGTC | | CCUG | | |
| 218 | CAGGCAAGGTTGGC | 463 | GAUAGUCGCCAACCUUG | 3241563:G > A | ALT |
| | GACTATC | | CCUG | | |
| 219 | AGGCAAGGTTGGCG | 464 | UGACAGUCGCCAACCUU | 3241563:G > A | REF |
| | ACTGTCA | | GCCU | | |
| 220 | AGGCAAGGTTGGCG | 465 | UGAUAGUCGCCAACCUU | 3241563:G > A | ALT |
| | ACTATCA | | GCCU | | |
| 221 | GGCAAGGTTGGCGA | 466 | AUGACAGUCGCCAACCU | 3241563:G > A | REF |
| | CTGTCAT | | UGCC | | |
| 222 | GGCAAGGTTGGCGA | 467 | AUGAUAGUCGCCAACCU | 3241563:G > A | ALT |
| | CTATCAT | | UGCC | | |
| 223 | GCAAGGTTGGCGAC | 468 | CAUGAUAGUCGCCAACC | 3241563:G > A | ALT |
| | TATCATG | | UUGC | | |
| 224 | CAAGGTTGGCGACTA | 469 | ACAUGAUAGUCGCCAAC | 3241563:G > A | ALT |
| | TCATGT | | CUUG | | |
| 225 | AAGGTTGGCGACTGT | 470 | CACAUGACAGUCGCCAA | 3241563:G > A | REF |
| | CATGTG | | CCUU | | |
| 226 | AAGGTTGGCGACTAT | 471 | CACAUGAUAGUCGCCAA | 3241563:G > A | ALT |
| | CATGTG | | CCUU | | |
| 227 | AGGTTGGCGACTGTC | 472 | CCACAUGACAGUCGCCA | 3241563:G > A | REF |
| | ATGTGG | | ACCU | | |
| 228 | AGGTTGGCGACTATC | 473 | CCACAUGAUAGUCGCCA | 3241563:G > A | ALT |
| | ATGTGG | | ACCU | | |
| 229 | GGTTGGCGACTGTCA | 474 | GCCACAUGACAGUCGCC | 3241563:G > A | REF |
| | TGTGGC | | AACC | | |
| 230 | GGTTGGCGACTATCA | 475 | GCCACAUGAUAGUCGCC | 3241563:G > A | ALT |
| | TGTGGC | | AACC | | |
| 231 | GTTGGCGACTGTCAT | 476 | AGCCACAUGACAGUCGC | 3241563:G > A | REF |
| | GTGGCT | | CAAC | | |
| 232 | GTTGGCGACTATCAT | 477 | AGCCACAUGAUAGUCGC | 3241563:G > A | ALT |
| | GTGGCT | | CAAC | | |
| 233 | CCCCCGCTTCCTCCCT | 478 | CACAGAGGGAGGAAGC | 3243330:C > T | ALT |
| | CTGTG | | GGGGG | | |
| 234 | CCCGCTTCCTCCCTCT | 479 | CCCACAGAGGGAGGAAG | 3243330:C > T | ALT |
| | GTGGG | | CGGG | | |
| 235 | CCCGCTTCCTCCCTCT | 480 | CCCGCAGAGGGAGGAAG | 3243330:C > T | REF |
| | GCGGG | | CGGG | | |
| 236 | CCGCTTCCTCCCTCTG | 481 | CCCCGCAGAGGGAGGAA | 3243330:C > T | REF |
| | CGGGG | | GCGG | | |
| 237 | CGCTTCCTCCCTCTGT | 482 | UCCCCACAGAGGGAGGA | 3243330:C > T | ALT |
| | GGGGA | | AGCG | | |
| 238 | GCTTCCTCCCTCTGC | 483 | CUCCCCGCAGAGGGAGG | 3243330:C > T | REF |
| | GGGGAG | | AAGC | | |
| 239 | GCTTCCTCCCTCTGTG | 484 | CUCCCCACAGAGGGAGG | 3243330:C > T | ALT |
| | GGGAG | | AAGC | | |
| 240 | CTTCCTCCCTCTGCG | 485 | CCUCCCCGCAGAGGGAG | 3243330:C > T | REF |
| | GGGAGG | | GAAG | | |
| 241 | CTTCCTCCCTCTGTGG | 486 | CCUCCCCACAGAGGGAG | 3243330:C > T | ALT |
| | GGAGG | | GAAG | | |
| 242 | TTCCTCCCTCTGCGG | 487 | UCCUCCCCGCAGAGGGA | 3243330:C > T | REF |
| | GGAGGA | | GGAA | | |
| 243 | TTCCTCCCTCTGTGG | 488 | UCCUCCCCACAGAGGGA | 3243330:C > T | ALT |
| | GGAGGA | | GGAA | | |
| 244 | TCCTCCCTCTGCGGG | 489 | GUCCUCCCCGCAGAGGG | 3243330:C > T | REF |
| | GAGGAC | | AGGA | | |
| 245 | CCTCCCTCTGCGGGG | 490 | GGUCCUCCCCGCAGAGG | 3243330:C > T | REF |
| | AGGACC | | GAGG | | |

Although the sequences listed in Table 2 are written as DNA sequences, the corresponding sequences can be in the form of RNA as well.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (1), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety.

Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:
Sense: mN*mN*/i2FN/mN/i2FN/mN/i2FN/mN/i2FN/mN/i2FN/mN/i2FN/mN/i2FN/mN/i2FN/*mN*/32FN/
Antisense: /52FN/*/i2FN/*mN/i2FN/mN/i2FN/mN/i2FN/mN/i2FN/mN/i2FN/mN/i2FN/mN/i2FN/mN/i2FN/mN*N*N
   wherein: “N” is the base; “2F” is a 2′-F modification; “m” is a 2′-O-methyl modification, “I” is an internal base; and “*” is a phosphorothioate backbone linkage.

In any of the embodiments described herein, the inhibitory nucleic acid molecules may be administered, for example, as one- to two-hour i.v. infusions or s.c. injections. In any of the embodiments described herein, the inhibitory nucleic acid molecules may be administered at dose levels that range from about 50 mg to about 900 mg, from about 100 mg to about 800 mg, from about 150 mg to about 700 mg, or from about 175 mg to about 640 mg (2.5 to 9.14 mg/kg; 92.5 to 338 mg/m²—based on an assumption of a body weight of 70 kg and a conversion of mg/kg to mg/m² dose levels based on a mg/kg dose multiplier value of 37 for humans).

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In any of the embodiments described herein, the inhibitory nucleic acid molecules specifically detect and/or identify a target sequence. The inhibitory nucleic acid molecules described herein may have complete nucleotide sequence identity of contiguous nucleotides within the target nucleotide sequence, although inhibitory nucleic acid molecules differing from the target nucleotide sequence and that retain the ability to specifically detect and/or identify a target nucleotide sequence may be designed by conventional methods. The inhibitory nucleic acid molecules described herein may have about 80%, about 85%, about 90%, about 91%, about 92%, about 93%, about 94%, about 95%, about 96%, about 97%, about 98%, about 99%, or 100% sequence identity or complementarity with the nucleotide sequences shown in Table 2.

Detecting the presence or absence of any of the mtHTT variants described herein in a biological sample from a subject and/or determining whether a subject has an mtHTT variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In any of the methods described herein, the subject may also be administered a Huntington's disease therapeutic agent. In some embodiments, the Huntington's disease therapeutic agent comprises an antipsychotic used for movement and other symptoms. In some embodiments, the Huntington's disease therapeutic agent includes, but is not limited to, deutetrabenazine, fluphenazine, haloperidol, tetrabenazine, tiapride, valbenazine, risperidone, olanzapine, cysteamine (such as cysteamine bitartrate), and aripiprazole, or any combination thereof.

The present disclosure also provides compositions comprising a combination of any of the inhibitory nucleic acid molecules described herein and any of the Huntington's disease therapeutic agents described herein.

Administration of the Huntington's disease therapeutic agents and/or inhibitory nucleic acid molecules can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the Huntington's disease therapeutic agents and/or inhibitory nucleic acid molecules can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients, or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in Huntington's disease, a decrease/reduction in the severity of Huntington's disease (such as, for example, a reduction or inhibition of development of Huntington's disease), a decrease/reduction in symptoms and disease-related effects, delaying the onset of symptoms and disease-related effects, reducing the severity of symptoms of disease-related effects, reducing the number of symptoms and disease-related effects, reducing the latency of symptoms and disease-related effects, an amelioration of symptoms and disease-related effects, reducing secondary symptoms, reducing secondary infections, preventing relapse to Huntington's disease, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of Huntington's disease development/progression (such as, for example, a complete or partial avoidance/inhibition or a delay), and an increased survival time of the affected host animal, following administration of a therapeutic protocol. Treatment of Huntington's disease encompasses the treatment of a subject already diagnosed as having any form of Huntington's disease at any clinical stage or manifestation, the delay of the onset or evolution or aggravation or deterioration of the symptoms or signs of Huntington's disease, and/or preventing and/or reducing the severity of Huntington's disease.

In some embodiments, the inhibitory nucleic acid molecule and the Huntington's disease therapeutic agent are disposed within a pharmaceutical composition. In some embodiments, the inhibitory nucleic acid molecule is disposed within a first pharmaceutical composition and the Huntington's disease therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

The present disclosure provides methods of identifying a subject having Huntington's disease or at risk of developing Huntington's disease that is a candidate for treatment with an inhibitory nucleic acid molecule. The subject has a pathogenic mutant huntingtin (mtHTT) allele. The methods comprise performing or having performed a sequence analysis on a biological sample obtained from the subject. In some embodiments, the methods comprise determining from the sequence analysis or having determined from the sequence analysis whether the subject is heterozygous for a first mtHTT variant, wherein a first mtHTT variant targeted allele is in phase with the pathogenic mtHTT allele, wherein the first mtHTT variant targeted allele is the alternate allele of the first mtHTT variant and is in phase with the pathogenic mtHTT allele more often than any other allele in a population of subjects. Such a subject is a candidate for treatment with a first mtHTT variant alternate allele-specific inhibitory nucleic acid molecule when the subject is heterozygous for the first mtHTT variant targeted allele in phase with the pathogenic mtHTT allele. In some embodiments, for a subject that is homozygous for the first mtHTT variant, or heterozygous for the first mtHTT variant but the targeted allele is not in phase with the pathogenic mtHTT allele, the methods comprise determining from the sequence analysis or having determined from the sequence analysis whether the subject is heterozygous for a subsequent mtHTT variant in which the alternate allele or reference allele is in phase with the pathogenic mtHTT allele. Such a subject is a candidate for treatment with a subsequent mtHTT variant reference allele-specific inhibitory nucleic acid molecule or a subsequent mtHTT variant alternate allele-specific inhibitory nucleic acid molecule, whichever is in phase with the pathogenic mtHTT allele, when the subject is heterozygous for the subsequent mtHTT variant.

The present disclosure also provides methods of identifying a subject having Huntington's disease or at risk of developing Huntington's disease that is a candidate for treatment with an inhibitory nucleic acid molecule. The subject has a pathogenic mutant huntingtin (mtHTT) allele. The methods comprise performing or having performed a sequence analysis on a biological sample obtained from the subject. In some embodiments, the methods comprise determining from the sequence analysis or having determined from the sequence analysis whether the subject is heterozygous for a first mtHTT variant, wherein a first mtHTT variant targeted allele is in phase with the pathogenic mtHTT allele, wherein the first mtHTT variant targeted allele is the alternate allele of the first mtHTT variant and is in phase with the pathogenic mtHTT allele more often than any other allele in a population of subjects. Such a subject is a candidate for treatment with a first mtHTT variant alternate allele-specific inhibitory nucleic acid molecule when the subject is heterozygous for the first mtHTT variant targeted allele in phase with the pathogenic mtHTT allele. In some embodiments, for a subject that is homozygous for the first mtHTT variant, or heterozygous for the first mtHTT variant but the targeted allele is not in phase with the pathogenic mtHTT allele, determining from the sequence analysis or having determined from the sequence analysis whether the subject is heterozygous for a first subsequent mtHTT variant and heterozygous for a second subsequent mtHTT variant. Such a subject is a candidate for treatment with: a) a first subsequent mtHTT variant alternate allele-specific/second subsequent mtHTT variant alternate allele-specific inhibitory nucleic acid molecule to the subject that has both the first subsequent mtHTT variant alternate allele and the second subsequent mtHTT variant alternate allele in phase with the pathogenic mtHTT allele; b) a first subsequent mtHTT variant reference allele-specific/second subsequent mtHTT variant reference allele-specific inhibitory nucleic acid molecule to the subject that has both the first subsequent mtHTT variant reference allele and the second subsequent mtHTT variant reference allele in phase with the pathogenic mtHTT allele; c) a first subsequent mtHTT variant alternate allele-specific/second subsequent mtHTT variant reference allele-specific inhibitory nucleic acid molecule to the subject that has both the first subsequent mtHTT variant alternate allele and the second subsequent mtHTT variant reference allele in phase with the pathogenic mtHTT allele; or d) a first subsequent mtHTT variant reference allele-specific/second subsequent mtHTT variant alternate allele-specific inhibitory nucleic acid molecule to the subject that has both the first subsequent mtHTT variant reference allele and the second subsequent mtHTT variant alternate allele in phase with the pathogenic mtHTT allele.

Determining whether a subject has of any of the mtHTT variants described herein in a biological sample from a subject and/or determining whether a subject has of any of the mtHTT variants described herein can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

The present disclosure also provides methods of detecting the presence or absence of any of the mtHTT variants described herein (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting of any of the mtHTT variants described herein, preliminary processing designed to isolate or enrich the biological sample for the genomic DNA can be employed. A variety of techniques may be used for this purpose.

In some embodiments, detecting of any of the mtHTT variants described herein in a subject comprises performing a sequence analysis on a biological sample obtained from the subject to determine whether of any of the mtHTT variant genomic nucleic acid molecule in the biological sample, and/or any of the mtHTT variant mRNA molecule in the biological sample, and/or any of the mtHTT variant cDNA molecule produced from an mRNA molecule in the biological sample, is present in the sample.

In some embodiments, the methods of detecting the presence or absence of any of the mtHTT variants described herein (such as, for example, a genomic nucleic acid molecule, an mRNA molecule, and/or a cDNA molecule produced from an mRNA molecule) in a subject comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising of any of the mtHTT variants described herein, and if mRNA, optionally reverse transcribing the mRNA into cDNA. Such assays can comprise, for example determining the identity of these positions of the particular mtHTT variant described herein. In some embodiments, the method is an in vitro method.

In some embodiments, the determining step, detecting step, or sequence analysis comprises sequencing at least a portion of the nucleotide sequence of of any of the mtHTT variants described herein in the biological sample that comprises a genetic variation compared to the corresponding HTT reference molecule.

In some embodiments, the assay comprises sequencing the entire nucleic acid molecule. In some embodiments, only an mtHTT variant genomic nucleic acid molecule is analyzed. In some embodiments, only an mtHTT variant mRNA is analyzed. In some embodiments, only an mtHTT variant cDNA obtained from the mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an mtHTT variant genomic sequence, variant mRNA sequence, or variant cDNA sequence and not the corresponding HTT reference sequence under stringent conditions and determining whether hybridization has occurred.

In some embodiments, the determining step, detecting step, or sequence analysis comprises: a) amplifying at least a portion of the mtHTT variant nucleic acid molecule; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45 °C, followed by a wash of 2×SSC at 50 °C, are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30 °C for short probes (such as, for example, 10 to 50 nucleotides) and at least about 60 °C for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, primers, including alteration-specific primers, can be used in second generation sequencing or high throughput sequencing. In some instances, the primers, including alteration-specific primers, can be modified. In particular, the primers can comprise various modifications that are used at different steps of, for example, Massively Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

In the context of the disclosure “specifically hybridizes” means that the probe or primer (such as, for example, the alteration-specific probe or alteration-specific primer) does not hybridize to a nucleic acid sequence encoding an HTT reference genomic nucleic acid molecule, an HTT reference mRNA molecule, and/or an HTT reference cDNA molecule.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×his or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides inhibitory nucleic acid molecules for use in the treatment or prevention of Huntington's disease in a subject having a pathogenic mtHTT allele. In some embodiments, the subject is heterozygous for a first mtHTT variant, wherein a first mtHTT variant targeted allele is in phase with the pathogenic mtHTT allele, wherein the first mtHTT variant targeted allele is the alternate allele of the first mtHTT variant and is in phase with the pathogenic mtHTT allele more often than any other allele in a population of subjects, and the inhibitory nucleic acid molecule comprises a first mtHTT variant alternate allele-specific inhibitory nucleic acid molecule. Any of the first mtHTT variant alternate allele-specific inhibitory nucleic acid molecules described herein is included herein. Any of the first mtHTT variants described herein is included herein. In some embodiments, the subject is homozygous for the first mtHTT variant or heterozygous for the first mtHTT variant but the targeted allele is not in phase with the pathogenic mtHTT allele, and the subject is heterozygous for a subsequent mtHTT variant in which the alternate allele or reference allele is in phase with the pathogenic mtHTT allele, and the inhibitory nucleic acid molecule comprises a subsequent mtHTT variant reference allele-specific inhibitory nucleic acid molecule or a subsequent mtHTT variant alternate allele-specific inhibitory nucleic acid molecule, whichever is in phase with the pathogenic mtHTT allele. In some embodiments, the subject is homozygous for the subsequent mtHTT variant, and the subject is heterozygous for a further subsequent mtHTT variant in which the alternate allele or reference allele is in phase with the pathogenic mtHTT allele, and the inhibitory nucleic acid molecule comprises a further subsequent mtHTT variant reference allele-specific inhibitory nucleic acid molecule or a further subsequent mtHTT variant alternate allele-specific inhibitory nucleic acid molecule, whichever is in phase with the pathogenic mtHTT allele. Any of the subsequent mtHTT variant reference allele-specific inhibitory nucleic acid molecules, subsequent mtHTT variant alternate allele-specific inhibitory nucleic acid molecules, further subsequent mtHTT variant reference allele-specific inhibitory nucleic acid molecules, or further subsequent mtHTT variant alternate allele-specific inhibitory nucleic acid molecules described herein is included herein. Any of the subsequent mtHTT variants or further subsequent mtHTT variants described herein is included herein.

The present disclosure also provides inhibitory nucleic acid molecules in the preparation of a medicament for treating or preventing Huntington's disease in a subject having a pathogenic mtHTT allele. In some embodiments, the subject is heterozygous for a first mtHTT variant, wherein a first mtHTT variant targeted allele is in phase with the pathogenic mtHTT allele, wherein the first mtHTT variant targeted allele is the alternate allele of the first mtHTT variant and is in phase with the pathogenic mtHTT allele more often than any other allele in a population of subjects, and the inhibitory nucleic acid molecule comprises a first mtHTT variant alternate allele-specific inhibitory nucleic acid molecule. Any of the first mtHTT variant alternate allele-specific inhibitory nucleic acid molecules described herein is included herein. Any of the first mtHTT variants described herein is included herein. In some embodiments, the subject is homozygous for the first mtHTT variant or heterozygous for the first mtHTT variant but the targeted allele is not in phase with the pathogenic mtHTT allele, and the subject is heterozygous for a subsequent mtHTT variant in which the alternate allele or reference allele is in phase with the pathogenic mtHTT allele, and the inhibitory nucleic acid molecule comprises a subsequent mtHTT variant reference allele-specific inhibitory nucleic acid molecule or a subsequent mtHTT variant alternate allele-specific inhibitory nucleic acid molecule, whichever is in phase with the pathogenic mtHTT allele. In some embodiments, the subject is homozygous for the subsequent mtHTT variant, and the subject is heterozygous for a further subsequent mtHTT variant in which the alternate allele or reference allele is in phase with the pathogenic mtHTT allele, and the inhibitory nucleic acid molecule comprises a further subsequent mtHTT variant reference allele-specific inhibitory nucleic acid molecule or a further subsequent mtHTT variant alternate allele-specific inhibitory nucleic acid molecule, whichever is in phase with the pathogenic mtHTT allele. Any of the subsequent mtHTT variant reference allele-specific inhibitory nucleic acid molecules, subsequent mtHTT variant alternate allele-specific inhibitory nucleic acid molecules, further subsequent mtHTT variant reference allele-specific inhibitory nucleic acid molecules, or further subsequent mtHTT variant alternate allele-specific inhibitory nucleic acid molecules described herein is included herein. Any of the subsequent mtHTT variants or further subsequent mtHTT variants described herein is included herein.

The present disclosure also provides inhibitory nucleic acid molecules for use in the treatment or prevention of Huntington's disease in a subject having a pathogenic mtHTT allele. In some embodiments, the subject is heterozygous for a first mtHTT variant, wherein the first mtHTT variant targeted allele is in phase with the pathogenic mtHTT allele, wherein the first mtHTT variant targeted allele is the alternate allele of the first mtHTT variant and is in phase with the pathogenic mtHTT allele more often than any other allele in a population of subjects, and the inhibitory nucleic acid molecule comprises a first mtHTT variant alternate allele-specific inhibitory nucleic acid molecule. Any of the first mtHTT variant alternate allele-specific inhibitory nucleic acid molecules described herein is included herein. Any of the first mtHTT variants described herein is included herein. In some embodiments, the subject is homozygous for the first mtHTT variant or heterozygous for the first mtHTT variant but the targeted allele is not in phase with the pathogenic mtHTT allele, and the subject is heterozygous for a first subsequent mtHTT variant and heterozygous for a second subsequent mtHTT variant, wherein the subject has both the first subsequent mtHTT variant alternate allele and the second subsequent mtHTT variant alternate allele in phase with the pathogenic mtHTT allele, and the inhibitory nucleic acid molecule comprises a first subsequent mtHTT variant alternate allele-specific/second subsequent mtHTT variant alternate allele-specific inhibitory nucleic acid molecule. In some embodiments, the subject is homozygous for the first mtHTT variant or heterozygous for the first mtHTT variant but the targeted allele is not in phase with the pathogenic mtHTT allele, and the subject is heterozygous for a first subsequent mtHTT variant and heterozygous for a second subsequent mtHTT variant, wherein the subject has both the first subsequent mtHTT variant reference allele and the second subsequent mtHTT variant reference allele in phase with the pathogenic mtHTT allele, and the inhibitory nucleic acid molecule comprises a first subsequent mtHTT variant reference allele-specific/second subsequent mtHTT variant reference allele-specific inhibitory nucleic acid molecule. In some embodiments, the subject is homozygous for the first mtHTT variant or heterozygous for the first mtHTT variant but the targeted allele is not in phase with the pathogenic mtHTT allele, and the subject is heterozygous for a first subsequent mtHTT variant and heterozygous for a second subsequent mtHTT variant, wherein the subject has both the first subsequent mtHTT variant alternate allele and the second subsequent mtHTT variant reference allele in phase with the pathogenic mtHTT allele, and the inhibitory nucleic acid molecule comprises a first subsequent mtHTT variant alternate allele-specific/second subsequent mtHTT variant reference allele-specific inhibitory nucleic acid molecule. In some embodiments, the subject is homozygous for the first mtHTT variant or heterozygous for the first mtHTT variant but the targeted allele is not in phase with the pathogenic mtHTT allele, and the subject is heterozygous for a first subsequent mtHTT variant and heterozygous for a second subsequent mtHTT variant, wherein the subject has both the first subsequent mtHTT variant reference allele and the second subsequent mtHTT variant alternate allele in phase with the pathogenic mtHTT allele, and the inhibitory nucleic acid molecule comprises a first subsequent mtHTT variant reference allele-specific/second subsequent mtHTT variant alternate allele-specific inhibitory nucleic acid molecule. Any of the first subsequent mtHTT variant alternate allele-specific/second subsequent mtHTT variant alternate allele-specific inhibitory nucleic acid molecules described herein is included herein. Any of the first subsequent mtHTT variant reference allele-specific/second subsequent mtHTT variant reference allele-specific inhibitory nucleic acid molecules described herein is included herein. Any of the first subsequent mtHTT variant alternate allele-specific/second subsequent mtHTT variant reference allele-specific inhibitory nucleic acid molecules described herein is included herein. Any of the first subsequent mtHTT variant reference allele-specific/second subsequent mtHTT variant alternate allele-specific inhibitory nucleic acid molecules described herein is included herein. Any of the first subsequent mtHTT variants and second subsequent mtHTT variants described herein are included herein.

The present disclosure also provides inhibitory nucleic acid molecules in the preparation of a medicament for treating or preventing Huntington's disease in a subject having a pathogenic mtHTT allele. In some embodiments, the subject is heterozygous for a first mtHTT variant, wherein the first mtHTT variant targeted allele is in phase with the pathogenic mtHTT allele, wherein the first mtHTT variant targeted allele is the alternate allele of the first mtHTT variant and is in phase with the pathogenic mtHTT allele more often than any other allele in a population of subjects, and the inhibitory nucleic acid molecule comprises a first mtHTT variant alternate allele-specific inhibitory nucleic acid molecule. Any of the first mtHTT variant alternate allele-specific inhibitory nucleic acid molecules described herein is included herein. Any of the first mtHTT variants described herein is included herein. In some embodiments, the subject is homozygous for the first mtHTT variant or heterozygous for the first mtHTT variant but the targeted allele is not in phase with the pathogenic mtHTT allele, and the subject is heterozygous for a first subsequent mtHTT variant and heterozygous for a second subsequent mtHTT variant, wherein the subject has both the first subsequent mtHTT variant alternate allele and the second subsequent mtHTT variant alternate allele in phase with the pathogenic mtHTT allele, and the inhibitory nucleic acid molecule comprises a first subsequent mtHTT variant alternate allele-specific/second subsequent mtHTT variant alternate allele-specific inhibitory nucleic acid molecule. In some embodiments, the subject is homozygous for the first mtHTT variant or heterozygous for the first mtHTT variant but the targeted allele is not in phase with the pathogenic mtHTT allele, and the subject is heterozygous for a first subsequent mtHTT variant and heterozygous for a second subsequent mtHTT variant, wherein the subject has both the first subsequent mtHTT variant reference allele and the second subsequent mtHTT variant reference allele in phase with the pathogenic mtHTT allele, and the inhibitory nucleic acid molecule comprises a first subsequent mtHTT variant reference allele-specific/second subsequent mtHTT variant reference allele-specific inhibitory nucleic acid molecule. In some embodiments, the subject is homozygous for the first mtHTT variant or heterozygous for the first mtHTT variant but the targeted allele is not in phase with the pathogenic mtHTT allele, and the subject is heterozygous for a first subsequent mtHTT variant and heterozygous for a second subsequent mtHTT variant, wherein the subject has both the first subsequent mtHTT variant alternate allele and the second subsequent mtHTT variant reference allele in phase with the pathogenic mtHTT allele, and the inhibitory nucleic acid molecule comprises a first subsequent mtHTT variant alternate allele-specific/second subsequent mtHTT variant reference allele-specific inhibitory nucleic acid molecule. In some embodiments, the subject is homozygous for the first mtHTT variant or heterozygous for the first mtHTT variant but the targeted allele is not in phase with the pathogenic mtHTT allele, and the subject is heterozygous for a first subsequent mtHTT variant and heterozygous for a second subsequent mtHTT variant, wherein the subject has both the first subsequent mtHTT variant reference allele and the second subsequent mtHTT variant alternate allele in phase with the pathogenic mtHTT allele, and the inhibitory nucleic acid molecule comprises a first subsequent mtHTT variant reference allele-specific/second subsequent mtHTT variant alternate allele-specific inhibitory nucleic acid molecule. Any of the first subsequent mtHTT variant alternate allele-specific/second subsequent mtHTT variant alternate allele-specific inhibitory nucleic acid molecules described herein is included herein. Any of the first subsequent mtHTT variant reference allele-specific/second subsequent mtHTT variant reference allele-specific inhibitory nucleic acid molecules described herein is included herein. Any of the first subsequent mtHTT variant alternate allele-specific/second subsequent mtHTT variant reference allele-specific inhibitory nucleic acid molecules described herein is included herein. Any of the first subsequent mtHTT variant reference allele-specific/second subsequent mtHTT variant alternate allele-specific inhibitory nucleic acid molecules described herein is included herein. Any of the first subsequent mtHTT variants and second subsequent mtHTT variants described herein are included herein.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in °C or is at ambient temperature, and pressure is at or near atmospheric.

### EXAMPLES

### Example 1: Variants Tagging HTT TRE in HD Cases

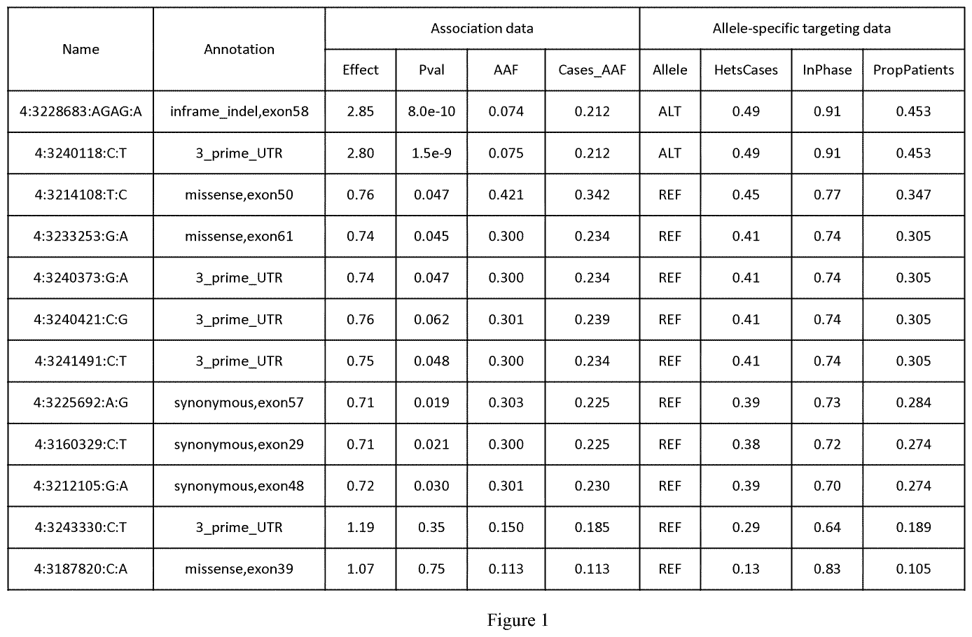

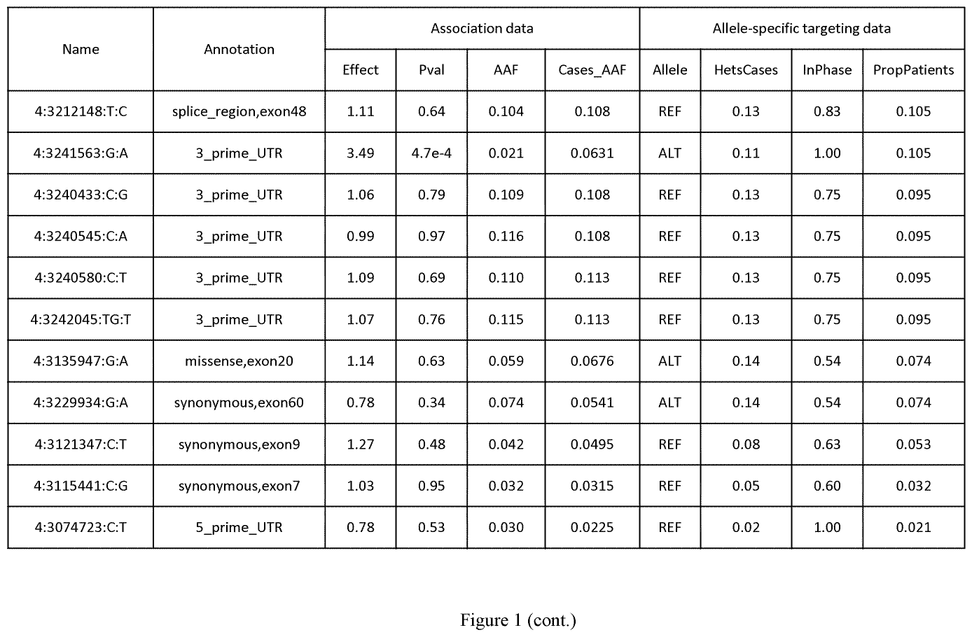

Twenty-three (23) HTT coding variants with MAF>1% (population) and >2% proportion of patients treatable were discovered (see, FIG. 1). Briefly, all variants identified in exome sequencing at Regeneron Genetics Center (RGC) mapping to human chromosome 4 positions 28-35 Mb were annotated with respect to the Huntingtin gene mRNA (coding exons, and 5′ and 3′ untranslated regions), and their frequencies in RGC samples were tabulated. In addition, the proportion of patients treatable was calculated for each common coding variant as follows. First an HTT region genotype dataset was constructed including subjects with ICD10 Huntington's disease (HD) diagnosis code G10 (cases) and control subjects without HD diagnosis from the same cohorts, and including (a) pseudo-genotypes for the HD pathogenic triplet repeat variant coded as heterozygous in cases (variant call format (VCF) “0/1”) and homozygous REF (“0/0”) in controls, (b) genotypes of HTT coding variants from exome sequencing, and (c) 24,393 variants from chromosome 4 positions 28-35 Mb derived by imputation from the TOPMED reference panel (Taliun et al., Nature, 2019, 590, 290-299; and Das et al., Nat. Genet., 2016, 48, 1284-1287). This HTT region dataset was then phased using the SHAPEIT software (Delaneau et al., Nature Commun., 2014, 5, 3934) to determine which alleles of HTT variants are on the same chromosome as the pathogenic allele in cases. For each HTT coding variant with minor allele frequency (MAF) greater than 0.01, we then calculated the proportion of HD cases that were heterozygous for the variant and the proportions of heterozygous individuals with ALT and REF alleles of the variant on the same chromosome as the HD pathogenic allele. The proportion of patients treatable by an allele-specific nucleic acid sequence for the variant/Alt allele or reference allele was calculated as the product of the proportion of heterozygous cases and the proportion of those with the given allele on the same chromosome as the HD pathogenic allele, respectively (shown in Table 1). Conditional proportions of patients treatable by targeting specific variants (Alt or reference alleles) was further calculated, conditional on first targeting another variant, by focusing the calculations of proportions of heterozygous cases and of specific alleles on the same chromosome as the pathogenic allele on HD cases who were not targetable by the conditioned-upon variant (i.e., HD cases who were not heterozygous for the conditioned-upon variant or who did not have the conditioned-upon variant's targeted allele on the same chromosome as the HD pathogenic allele). Conditional proportions of patients treated (not shown in Table 1 as they depend on the sequence of conditioned-upon variant(s)) allowed for optimization of the choice of second variants/alleles to target in HD patients.

### Example 2: Design of Candidate Oligonucleotides for HTT

Targetable variants were cross-referenced with candidate oligos. Candidate oligonucleotides were designed based on the HTT reference sequence. 2,667 candidate oligonucleotides were identified throughout HTT. 14 oligonucleotide cadidates 5′ of the pathogenic TRE site were identified. Zero oligonucleotides overlapping the pathogenic TRE site 4:4:3074877:CAG:(CAG)ₙ were identified. 2,650 additional oligonucleotides in exons 2 to 67, and >600 oligos in the 3′ UTR were identified. 48 candidate oligonucleotides that overlap 5 common HTT coding variants were identified. 135 candidate oligonucleotides overlapping HTT variants in the 3′ UTR were identified. A list of oligonucleotides is shown in Table 2. A set of 11 preferred oligonucleotides (sense sequence/target antisense sequence) includes SEQ ID Nos: 88/333, 89/334, 90/335, 91/336,135/380, 137/382, 139/384, 142/387, 144/389, 147/392, and 149/394.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1.-85. (canceled)

86. A method of treating a subject having Huntington's disease or at risk of developing Huntington's disease, wherein the subject has a pathogenic mutant huntingtin (mtHTT) allele, the method comprising:
a) performing or having performed a sequence analysis on a biological sample obtained from the subject;
b) determining from the sequence analysis or having determined from the sequence analysis whether the subject is heterozygous for a first mtHTT variant, wherein a first mtHTT variant targeted allele is in phase with the pathogenic mtHTT allele, wherein the first mtHTT variant targeted allele is the alternate allele of the first mtHTT variant and is in phase with the pathogenic mtHTT allele more often than any other allele in a population of subjects; and
i) administering or continuing to administer a first mtHTT variant alternate allele-specific inhibitory nucleic acid molecule to the subject that is heterozygous for the first mtHTT variant targeted allele in phase with the pathogenic mtHTT allele; or
ii) performing step c) for the subject that is homozygous for the first mtHTT variant or heterozygous for the first mtHTT variant but the targeted allele is not in phase with the pathogenic mtHTT allele;
c) determining from the sequence analysis or having determined from the sequence analysis whether the subject is heterozygous for a subsequent mtHTT variant in which the alternate allele or reference allele is in phase with the pathogenic mtHTT allele; and
i) administering or continuing to administer a subsequent mtHTT variant reference allele-specific inhibitory nucleic acid molecule or a subsequent mtHTT variant alternate allele-specific inhibitory nucleic acid molecule, whichever is in phase with the pathogenic mtHTT allele, to the subject that is heterozygous for the subsequent mtHTT variant; or
ii) repeating step c) for the subject that is homozygous for the subsequent mtHTT variant until a further subsequent mtHTT variant that is heterozygous is identified for the subject, and administering to the subject or continuing to administer to the subject a further subsequent mtHTT variant reference allele-specific inhibitory nucleic acid molecule or a further subsequent mtHTT variant alternate allele-specific inhibitory nucleic acid molecule, whichever is in phase with the pathogenic mtHTT allele.

87. The method of claim 86, wherein the first mtHTT variant alternate allele-specific inhibitory nucleic acid molecule, subsequent mtHTT variant reference allele-specific inhibitory nucleic acid molecule, subsequent mtHTT variant alternate allele-specific inhibitory nucleic acid molecule, further subsequent mtHTT variant reference allele-specific inhibitory nucleic acid molecule, and further subsequent mtHTT variant alternate allele-specific inhibitory nucleic acid molecule comprise an antisense nucleic acid molecule, a small interfering RNA (siRNA), or a short hairpin RNA (shRNA).

88. The method of claim 87, wherein the first mtHTT variant alternate allele-specific inhibitory nucleic acid molecule, subsequent mtHTT variant reference allele-specific inhibitory nucleic acid molecule, subsequent mtHTT variant alternate allele-specific inhibitory nucleic acid molecule, further subsequent mtHTT variant reference allele-specific inhibitory nucleic acid molecule, and further subsequent mtHTT variant alternate allele-specific inhibitory nucleic acid molecule comprise an siRNA.

89. The method of claim 87, wherein the first mtHTT variant alternate allele-specific inhibitory nucleic acid molecule, subsequent mtHTT variant reference allele-specific inhibitory nucleic acid molecule, subsequent mtHTT variant alternate allele-specific inhibitory nucleic acid molecule, further subsequent mtHTT variant reference allele-specific inhibitory nucleic acid molecule, and further subsequent mtHTT variant alternate allele-specific inhibitory nucleic acid molecule comprise an antisense nucleic acid molecule.

90. The method of claim 86, wherein the first mtHTT variant comprises 4:3228683:AGAG:A; and i) the first mtHTT variant alternate-specific inhibitory nucleic acid molecule comprises an antisense nucleic acid molecule comprising the nucleotide sequence of SEQ ID NO:334, SEQ ID NO:335, or SEQ ID NO:336; or ii) the first mtHTT variant alternate-specific inhibitory nucleic acid molecule comprises an siRNA comprising the nucleotide sequences of SEQ ID NOs:89/334, SEQ ID NOs:90/335, or SEQ ID NOs:91/336.

91. The method of claim 86, wherein the subsequent mtHTT variant or further subsequent mtHTT variant comprises 4:3240421:C:G; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:380, SEQ ID NO:382, SEQ ID NO:384, SEQ ID NO:387, SEQ ID NO:389, or SEQ ID NO:392; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:135/380, SEQ ID NOs:137/382, SEQ ID NOs:139/384, SEQ ID NOs:142/387, SEQ ID NOs:144/389, or SEQ ID NOs:147/392; iii) the subsequent mtHTT variant reference-specific inhibitory nucleic acid molecule or further subsequent mtHTT variant reference-specific inhibitory nucleic acid molecule comprises an antisense nucleic acid molecule comprising the nucleotide sequence of SEQ ID NO:379, SEQ ID NO:381, SEQ ID NO:383, SEQ ID NO:385, SEQ ID NO:386, SEQ ID NO:388, SEQ ID NO:390, SEQ ID NO:391, or SEQ ID NO:393; or iv) the subsequent mtHTT variant reference-specific inhibitory nucleic acid molecule or further subsequent mtHTT variant reference-specific inhibitory nucleic acid molecule comprises an siRNA comprising the nucleotide sequences of SEQ ID NOs:134/379, SEQ ID NOs:136/381, SEQ ID NOs:138/383, SEQ ID NOs:140/385, SEQ ID NOs:141/386, SEQ ID NOs:143/388, SEQ ID NOs:145/390, SEQ ID NOs:146/391, or SEQ ID NOs:148/393.

92. The method of claim 86, wherein the subsequent mtHTT variant or further subsequent mtHTT variant comprises 4:3074723:C:T; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:247, SEQ ID NO:249, SEQ ID NO:251, SEQ ID NO:253, SEQ ID NO:255, SEQ ID NO:257, SEQ ID NO:259, SEQ ID NO:261, or SEQ ID NO:263; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:2/247, SEQ ID NOs:4/249, SEQ ID NOs:6/251, SEQ ID NOs:8/253, SEQ ID NOs:10/255, SEQ ID NOs:12/257, SEQ ID NOs:14/259, SEQ ID NOs:16/261, or SEQ ID NOs:18/263; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:246, SEQ ID NO:248, SEQ ID NO:250, SEQ ID NO:252, SEQ ID NO:254, SEQ ID NO:256, SEQ ID NO:258, SEQ ID NO:260, or SEQ ID NO:262; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:1/246, SEQ ID NOs:3/248, SEQ ID NOs:5/250, SEQ ID NOs:7/252, SEQ ID NOs:9/254, SEQ ID NOs:11/256, SEQ ID NOs:13/258, SEQ ID NOs:15/260, or SEQ ID NOs:17/262.

93. The method of claim 86, wherein the subsequent mtHTT variant or further subsequent mtHTT variant comprises 4:3115441:C:G; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:265, SEQ ID NO:266, SEQ ID NO:268, SEQ ID NO:270, SEQ ID NO:272, SEQ ID NO:274, SEQ ID NO:276, or SEQ ID NO:278; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:20/265, SEQ ID NOs:21/266, SEQ ID NOs:23/268, SEQ ID NOs:25/270, SEQ ID NOs:27/272, SEQ ID NOs:29/274, SEQ ID NOs:31/276, or SEQ ID NOs:33/278; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:264, SEQ ID NO:267, SEQ ID NO:269, SEQ ID NO:271, SEQ ID NO:273, SEQ ID NO:275, or SEQ ID NO:277; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:19/264, SEQ ID NOs:22/267, SEQ ID NOs:24/269, SEQ ID NOs:26/271, SEQ ID NOs:28/273, SEQ ID NOs:30/275, or SEQ ID NOs:32/277.

94. The method of claim 86, wherein the subsequent mtHTT variant or further subsequent mtHTT variant comprises 4:3160329:C:T; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:279, SEQ ID NO:280, SEQ ID NO:282, SEQ ID NO:284, SEQ ID NO:286, or SEQ ID NO:287; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:34/279, SEQ ID NOs:35/280, SEQ ID NOs:37/282, SEQ ID NOs:39/284, SEQ ID NOs:41/286, or SEQ ID NOs:42/287; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:281, SEQ ID NO:283, SEQ ID NO:285, or SEQ ID NO:288; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:36/281, SEQ ID NOs:38/283, SEQ ID NOs:40/285, or SEQ ID NOs:43/288.

95. The method of claim 86, wherein the subsequent mtHTT variant or further subsequent mtHTT variant comprises 4:3187820:C:A; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:289, SEQ ID NO:291, SEQ ID NO:294, SEQ ID NO:296, or SEQ ID NO:297; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:44/289, SEQ ID NOs:46/291, SEQ ID NOs:49/294, SEQ ID NOs:51/296, or SEQ ID NOs:52/297; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:290, SEQ ID NO:292, SEQ ID NO:293, SEQ ID NO:295, or SEQ ID NO:298; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:45/290, SEQ ID NOs:47/292, SEQ ID NOs:48/293, SEQ ID NOs:50/295, or SEQ ID NOs:53/298.

96. The method of claim 86, wherein the subsequent mtHTT variant or further subsequent mtHTT variant comprises 4:3212105:G:A; and i) the subsequent mtHTT variant reference-specific inhibitory nucleic acid molecule or further subsequent mtHTT variant reference-specific inhibitory nucleic acid molecule comprises an antisense nucleic acid molecule comprising the nucleotide sequence of SEQ ID NO:299, SEQ ID NO:300, SEQ ID NO:301, SEQ ID NO:302, or SEQ ID NO:303; or ii) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:54/299, SEQ ID NOs:55/300, SEQ ID NOs:56/301, SEQ ID NOs:57/302, or SEQ ID NOs:58/303.

97. The method of claim 86, wherein the subsequent mtHTT variant or further subsequent mtHTT variant comprises 4:3229934:G:A; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:313, SEQ ID NO:315, SEQ ID NO:316, SEQ ID NO:317, SEQ ID NO:320, SEQ ID NO:322, or SEQ ID NO:325; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:68/313, SEQ ID NOs:70/315, SEQ ID NOs:71/316, SEQ ID NOs:72/317, SEQ ID NOs:75/320, SEQ ID NOs:77/322, or SEQ ID NOs:80/325; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:312, SEQ ID NO:314, SEQ ID NO:318, SEQ ID NO:319, SEQ ID NO:321, SEQ ID NO:323, or SEQ ID NO:324; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:67/312, SEQ ID NOs:69/314, SEQ ID NOs:73/318, SEQ ID NOs:74/319, SEQ ID NOs:76/321, SEQ ID NOs:78/323, or SEQ ID NOs:79/324.

98. The method of claim 86, wherein the subsequent mtHTT variant or further subsequent mtHTT variant comprises 4:3225692:A:G; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:326, SEQ ID NO:328, or SEQ ID NO:330; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:81/326, SEQ ID NOs:83/328, or SEQ ID NOs:85/330; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:327, SEQ ID NO:329, SEQ ID NO:331, or SEQ ID NO:332; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:82/327, SEQ ID NOs:84/329, SEQ ID NOs:86/331, or SEQ ID NOs:87/332.

99. The method of claim 86, wherein the subsequent mtHTT variant or further subsequent mtHTT variant comprises 4:3233253:G:A; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:338, SEQ ID NO:339, SEQ ID NO:341, SEQ ID NO:345, SEQ ID NO:346, SEQ ID NO:349, or SEQ ID NO:351; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:93/338, SEQ ID NOs:94/339, SEQ ID NOs:96/341, SEQ ID NOs:100/345, SEQ ID NOs:101/346, SEQ ID NOs:104/349, or SEQ ID NOs:106/351; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:337, SEQ ID NO:340, SEQ ID NO:342, SEQ ID NO:343, SEQ ID NO:344, SEQ ID NO:347, SEQ ID NO:348, or SEQ ID NO:350; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:92/337, SEQ ID NOs:95/340, SEQ ID NOs:97/342, SEQ ID NOs:98/343, SEQ ID NOs:99/344, SEQ ID NOs:102/347, SEQ ID NOs:103/348, or SEQ ID NOs:105/350.

100. The method of claim 86, wherein the subsequent mtHTT variant or further subsequent mtHTT variant comprises 4:3240373:G:A; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:370, SEQ ID NO:372, SEQ ID NO:373, SEQ ID NO:376, SEQ ID NO:377, or SEQ ID NO:378; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:125/370, SEQ ID NOs:127/372, SEQ ID NOs:128/373, SEQ ID NOs:131/376, SEQ ID NOs:132/377, or SEQ ID NOs:133/378; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:369, SEQ ID NO:371, SEQ ID NO:374, or SEQ ID NO:375; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:124/369, SEQ ID NOs:126/371, SEQ ID NOs:129/374, or SEQ ID NOs:130/375.

101. The method of claim 86, wherein the subsequent mtHTT variant or further subsequent mtHTT variant comprises 4:3240433:C:G; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:410 or SEQ ID NO:411; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:165/410 or SEQ ID NOs:166/411; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:409 or SEQ ID NO:412; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:164/409 or SEQ ID NOs:167/412.

102. The method of claim 86, wherein the subsequent mtHTT variant or further subsequent mtHTT variant comprises 4:3240545:C:A; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:413, SEQ ID NO:415, SEQ ID NO:417, SEQ ID NO:419, SEQ ID NO:421, SEQ ID NO:422, or SEQ ID NO:424; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:168/413, SEQ ID NOs:170/415, SEQ ID NOs:172/417, SEQ ID NOs:174/419, SEQ ID NOs:176/421, SEQ ID NOs:177/422, or SEQ ID NOs:179/424; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:414, SEQ ID NO:416, SEQ ID NO:418, SEQ ID NO:420, or SEQ ID NO:423; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:169/414, SEQ ID NOs:171/416, SEQ ID NOs:173/418, SEQ ID NOs:175/420, or SEQ ID NOs:178/423.

103. The method of claim 86, wherein the subsequent mtHTT variant or further subsequent mtHTT variant comprises 4:3240580:C:T; and i) the subsequent mtHTT variant alternate-specific inhibitory nucleic acid molecule or further subsequent mtHTT variant alternate-specific inhibitory nucleic acid molecule comprises an antisense nucleic acid molecule comprising the nucleotide sequence of SEQ ID NO:426, SEQ ID NO:430, SEQ ID NO:432, SEQ ID NO:433, SEQ ID NO:436, SEQ ID NO:438, SEQ ID NO:439, or SEQ ID NO:441; ii) the subsequent mtHTT variant alternate-specific inhibitory nucleic acid molecule or further subsequent mtHTT variant alternate-specific inhibitory nucleic acid molecule comprises an siRNA comprising the nucleotide sequences of SEQ ID NOs:181/426, SEQ ID NOs:185/430, SEQ ID NOs:187/432, SEQ ID NOs:188/433, SEQ ID NOs:191/436, SEQ ID NOs:193/438, SEQ ID NOs:194/439, or SEQ ID NOs:196/441; iii) the subsequent mtHTT reference-specific inhibitory nucleic acid molecule or further subsequent mtHTT reference-specific inhibitory nucleic acid molecule comprises an antisense nucleic acid molecule comprising the nucleotide sequence of SEQ ID NO:425, SEQ ID NO:427, SEQ ID NO:428, SEQ ID NO:429, SEQ ID NO:431, SEQ ID NO:434, SEQ ID NO:435, SEQ ID NO:437, SEQ ID NO:440, or SEQ ID NO:442; or iv) the subsequent mtHTT reference-specific inhibitory nucleic acid molecule or further subsequent mtHTT reference-specific inhibitory nucleic acid molecule comprises an siRNA comprising the nucleotide sequences of SEQ ID NOs:180/425, SEQ ID NOs:182/427, SEQ ID NOs:183/428, SEQ ID NOs:184/429, SEQ ID NOs:186/431, SEQ ID NOs:189/434, SEQ ID NOs:190/435, SEQ ID NOs:192/437, SEQ ID NOs:195/440, or SEQ ID NOs:197/442.

104. A method of treating a subject having Huntington's disease or at risk of developing Huntington's disease, wherein the subject has a pathogenic mutant huntingtin (mtHTT) allele, the method comprising:
a) performing or having performed a sequence analysis on a biological sample obtained from the subject;
b) determining from the sequence analysis or having determined from the sequence analysis whether the subject is heterozygous for a first mtHTT variant, wherein the first mtHTT variant targeted allele is in phase with the pathogenic mtHTT allele, wherein the first mtHTT variant targeted allele is the alternate allele of the first mtHTT variant and is in phase with the pathogenic mtHTT allele more often than any other allele in a population of subjects; and
i) administering or continuing to administer a first mtHTT variant alternate allele-specific inhibitory nucleic acid molecule to the subject that is heterozygous for the first mtHTT variant targeted allele in phase with the pathogenic mtHTT allele; or
ii) performing step c) for the subject that is homozygous for the first mtHTT variant or heterozygous for the first mtHTT variant but the targeted allele is not in phase with the pathogenic mtHTT allele;
c) determining from the sequence analysis or having determined from the sequence analysis whether the subject is heterozygous for a first subsequent mtHTT variant and heterozygous for a second subsequent mtHTT variant; and
i) administering or continuing to administer a first subsequent mtHTT variant alternate allele-specific/second subsequent mtHTT variant alternate allele-specific inhibitory nucleic acid molecule to the subject that has both the first subsequent mtHTT variant alternate allele and the second subsequent mtHTT variant alternate allele in phase with the pathogenic mtHTT allele; or
ii) administering or continuing to administer a first subsequent mtHTT variant reference allele-specific/second subsequent mtHTT variant reference allele-specific inhibitory nucleic acid molecule to the subject that has both the first subsequent mtHTT variant reference allele and the second subsequent mtHTT variant reference allele in phase with the pathogenic mtHTT allele; or
iii) administering or continuing to administer a first subsequent mtHTT variant alternate allele-specific/second subsequent mtHTT variant reference allele-specific inhibitory nucleic acid molecule to the subject that has both the first subsequent mtHTT variant alternate allele and the second subsequent mtHTT variant reference allele in phase with the pathogenic mtHTT allele; or
iv) administering or continuing to administer a first subsequent mtHTT variant reference allele-specific/second subsequent mtHTT variant alternate allele-specific inhibitory nucleic acid molecule to the subject that has both the first subsequent mtHTT variant reference allele and the second subsequent mtHTT variant alternate allele in phase with the pathogenic mtHTT allele; or
v) repeating step c) for the subject that is homozygous for either the first subsequent mtHTT variant or the second subsequent mtHTT variant until a first further subsequent mtHTT variant a second further subsequent mtHTT variant that are heterozygous is identified for the subject, and administering to the subject or continuing to administer to the subject:
a first further subsequent mtHTT variant alternate allele-specific/second further subsequent mtHTT variant alternate allele-specific inhibitory nucleic acid molecule to the subject that has both the first further subsequent mtHTT variant alternate allele and the second further subsequent mtHTT variant alternate allele in phase with the pathogenic mtHTT allele; or
a first further subsequent mtHTT variant reference allele-specific/second further subsequent mtHTT variant reference allele-specific inhibitory nucleic acid molecule to the subject that has both the first further subsequent mtHTT variant reference allele and the second further subsequent mtHTT variant reference allele in phase with the pathogenic mtHTT allele; or
a first further subsequent mtHTT variant alternate allele-specific/second further subsequent mtHTT variant reference allele-specific inhibitory nucleic acid molecule to the subject that has both the first further subsequent mtHTT variant alternate allele and the second further subsequent mtHTT variant reference allele in phase with the pathogenic mtHTT allele; or
a first further subsequent mtHTT variant reference allele-specific/second further subsequent mtHTT variant alternate allele-specific inhibitory nucleic acid molecule to the subject that has both the first further subsequent mtHTT variant reference allele and the second further subsequent mtHTT variant alternate allele in phase with the pathogenic mtHTT allele.

105. A method of identifying a subject having Huntington's disease or at risk of developing Huntington's disease that is a candidate for treatment with an inhibitory nucleic acid molecule, wherein the subject has a pathogenic mutant huntingtin (mtHTT) allele, the method comprising:
a) performing or having performed a sequence analysis on a biological sample obtained from the subject; and
i) determining from the sequence analysis or having determined from the sequence analysis whether the subject is heterozygous for a first mtHTT variant, wherein a first mtHTT variant targeted allele is in phase with the pathogenic mtHTT allele, wherein the first mtHTT variant targeted allele is the alternate allele of the first mtHTT variant and is in phase with the pathogenic mtHTT allele more often than any other allele in a population of subjects, wherein the subject is a candidate for treatment with a first mtHTT variant alternate allele-specific inhibitory nucleic acid molecule when the subject is heterozygous for the first mtHTT variant targeted allele in phase with the pathogenic mtHTT allele; or
ii) for the subject that is homozygous for the first mtHTT variant, or heterozygous for the first mtHTT variant but the targeted allele is not in phase with the pathogenic mtHTT allele, determining from the sequence analysis or having determined from the sequence analysis whether the subject is heterozygous for a subsequent mtHTT variant in which the alternate allele or reference allele is in phase with the pathogenic mtHTT allele, wherein the subject is a candidate for treatment with a subsequent mtHTT variant reference allele-specific inhibitory nucleic acid molecule or a subsequent mtHTT variant alternate allele-specific inhibitory nucleic acid molecule, whichever is in phase with the pathogenic mtHTT allele, when the subject is heterozygous for the subsequent mtHTT variant.
